(19) United States
(12) Patent Application Publication
(10) Pub. No.: US 2024/0335451 A1
(43) Pub. Date: Oct. 10, 2024

(54) ACRYLAMIDE DERIVATIVES USEFUL AS ANTI-INFLAMMATORY AGENTS

(71) Applicant: Sitryx Therapeutics Limited, Oxford (GB)

(72) Inventors: Matthew Colin Thor FYFE, Oxford (GB); Alessandro MAZZACANI, Oxford (GB); Barry John TEOBALD, Oxford (GB); Michael Liam COOKE, Nottingham, Nottinghamshire (GB); Saleh AHMED, Nottingham, Nottinghamshire (GB)

(73) Assignee: Sitryx Therapeutics Limited, Oxford (GB)

(21) Appl. No.: 18/572,126

(22) PCT Filed: Jun. 22, 2022
(86) PCT No.: PCT/GB2022/051582

§ 371 Date: Dec. 19, 2023

(30) Foreign Application Priority Data
Jun. 22, 2021 (EP) 21180920.7

(51) Int. Cl. A61K 31/5375; A61K 31/165; A61K 31/337; A61K 31/38; A61K 31/427; A61K 31/4439; A61K 31/501; A61K 45/06; A61P 29/00; A61P 37/06; C07C 235/76; C07D 211/58; C07D 213/30; C07D 241/20; C07D 271/06; C07D 305/08; C07D 331/04; C07D 413/12

(52) U.S. Cl. CPC A61K 31/5375; A61K 31/165; A61K 31/337; A61K 31/38; A61K 31/427; A61K 31/4439; A61K 31/501; A61K 45/06; A61P 29/00; A61P 37/06; C07C 235/76; C07D 211/58; C07D 213/30; C07D 241/20; C07D 271/06; C07D 305/08; C07D 331/04; C07D 413/12

(57) ABSTRACT

The invention relates to compounds of formula (I) and to their use in treating or preventing an inflammatory disease or a disease associated with an undesirable immune response:

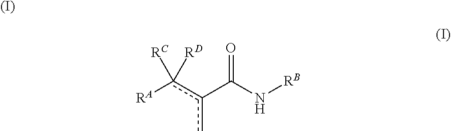

wherein R^{A}, R^{B}, R^{C} and R^{D} are as defined herein.

### FIELD OF THE INVENTION

The present invention relates to compounds and their use in treating or preventing inflammatory diseases or diseases associated with an undesirable immune response, and to related compositions, methods and intermediate compounds.

### BACKGROUND OF THE INVENTION

Chronic inflammatory diseases such as rheumatoid arthritis, systemic lupus erythematosus (SLE), multiple sclerosis, psoriasis, Crohn's disease, ulcerative colitis, uveitis and chronic obstructive pulmonary disease (COPD) represent a significant burden to society because of life-long debilitating illness, increased mortality and high costs for therapy and care (Straub R. H. and Schradin C., 2016). Non-steroidal anti-inflammatory drugs (NSAIDs) are the most widespread medicines employed for treating inflammatory disorders, but these agents do not prevent the progression of the inflammation and only treat the accompanying symptoms. Glucocorticoids are powerful anti-inflammatory agents, making them emergency treatments for acute inflammatory flares, but given longer term these medicines give rise to a plethora of unwanted side-effects and may also be subject to resistance (Straub R. H. and Cutolo M., 2016). Thus, considerable unmet medical need still exists for the treatment of inflammatory disorders and extensive efforts to discover new medicines to alleviate the burden of these diseases is ongoing (Hanke T. et al., 2016).

Dimethyl fumarate (DMF), a diester of the citric acid cycle (CAC) intermediate fumaric acid, is utilised as an oral therapy for treating psoriasis (Brück J. et al., 2018) and multiple sclerosis (Mills E. A. et al., 2018). Importantly, following oral administration, none of this agent is detected in plasma (Dibbert S. et al., 2013), the only drug-related compounds observed being the hydrolysis product monomethyl fumarate (MMF) and glutathione (GSH) conjugates of both the parent (DMF) and metabolite (MMF). DMF's mechanism of action is complex and controversial. This compound's efficacy has been attributed to a multiplicity of different phenomena involving covalent modification of proteins and the conversion of “prodrug” DMF to MMF. In particular, the following pathways have been highlighted as being of relevance to DMF's anti-inflammatory effects: 1) activation of the anti-oxidant, anti-inflammatory, nuclear factor (erythroid-derived 2)-like 2 (NRF2) pathway as a consequence of reaction of the electrophilic α,β-unsaturated ester moiety with nucleophilic cysteine residues on kelch-like ECH-associated protein 1 (KEAP1) (Brennan M. S. et al., 2015); 2) induction of activating transcription factor 3 (ATF3), leading to suppression of pro-inflammatory cytokines interleukin (IL)-6 and IL-8 (Müller S. et al., 2017); 3) inactivation of the glycolytic enzyme glyceraldehyde 3-phosphate dehydrogenase (GAPDH) through succination of its catalytic cysteine residue with a Michael accepting unsaturated ester (Kornberg M. D. et al., 2018; Angiari S. and O'Neill L. A., 2018); 4) inhibition of nuclear factor-kappaB (NF-κB)-driven cytokine production (Gillard G. O. et al., 2015); 5) preventing the association of PKC6 with the costimulatory receptor CD28 to reduce the production of IL-2 and block T-cell activation (Blewett M. M. et al., 2016); 6) reaction of the electrophilic α,β-unsaturated ester with the nucleophilic thiol group of anti-oxidant GSH, impacting cellular responses to oxidative stress (Lehmann J. C. U. et al., 2007); 7) agonism of the hydroxycarboxylic acid receptor 2 (HCA2) by the MMF generated in vivo through DMF hydrolysis (von Glehn F. et al., 2018); 8) allosteric covalent inhibition of the p90 ribosomal S6 kinases (Andersen J. L. et al., 2018); 9) inhibition of the expression and function of hypoxia-inducible factor-1a (HIF-1a) and its target genes, such as IL-8 (Zhao G. et al., 2014); and 10) inhibition of Toll-like receptor (TLR)-induced M1 and K63 ubiquitin chain formation (McGuire V. A. et al., 2016). In general, with the exception of HCA2 agonism (Tang H. et al., 2008), membrane permeable diester DMF tends to exhibit much more profound biological effects in cells compared to its monoester counterpart MMF. However, the lack of systemic exposure of DMF in vivo has led some researchers to assert that MMF is, in fact, the principal active component following oral DMF administration (Mrowietz U. et al., 2018). As such, it is evident that some of the profound biology exerted by DMF in cells is lost because of hydrolysis in vivo to MMF.

Recently, it has been discovered that, during inflammatory macrophage activation, the CAC becomes anaplerotic and is diverted such that the unsaturated diacid itaconic acid, “itaconate”, is generated (Murphy M. P. and O'Neill L. A. J., 2018; O'Neill L. A. J. and Artyomov M. N., 2019; Yu X.-H. et al., 2019). Instead of being hydrated to isocitrate by aconitate hydratase, the CAC intermediate aconitate is decarboxylated by the protein product of immune-responsive gene 1 (IRG1), one of the most highly upregulated genes in macrophages under proinflammatory conditions, subsequently named aconitate decarboxylase 1, to produce itaconic acid (Michelucci A. et al., 2013). This unsaturated diacid is an inhibitor of the bacterial enzyme isocitrate lyase and, as such, it exerts anti-bacterial activity. In addition, itaconic acid has been shown to inhibit the CAC enzyme succinate dehydrogenase (SDH) (Ackermann et al., 1949), leading accordingly to succinate accumulation (Cordes T. et al., 2016). By inhibiting SDH, an enzyme critical for the inflammatory response (E. L. Mills et al., 2016), itaconate ameliorates inflammation in vitro and in vivo during macrophage activation and ischemia-reperfusion injury (Lampropoulou V. et al., 2016).

Like fumaric acid, itaconic acid is an α,β-unsaturated carboxylic acid. As such, it is a Michael acceptor which induces a global electrophilic stress response. In this regard, the itaconic acid diesterdimethyl itaconate (DM1), like DMF, produces an anti-inflammatory response, reducing the expression levels of pro-inflammatory cytokines IL-1β, IL-6, IL-12 and IL-18 in lipopolysaccharide (LPS)-stimulated bone marrow-derived macrophages (WO2017/142855A1, incorporated herein by reference). This response appears to be mediated, in part, by NRF2 activation, via alkylation of KEAP1 cysteine residues by the electrophilic α,β-unsaturated ester moiety (Mills E. L. et al., 2018), which enhances the expression of downstream genes with anti-oxidant and anti-inflammatory capacities. Nevertheless, not all of the pronounced immunoregulatory effects engendered by DMI can be attributed to NRF2 activation. In particular, the modulation of IκBζ by DMI is independent of NRF2 and is mediated via upregulation of ATF3, a global negative regulator of immune activation that downregulates various cytokines, such as IL-6 (Bambouskova M. et al., 2018). Moreover, by inhibiting IκBζ protein production, DMI ameliorates IL-17-mediated pathologies, highlighting the therapeutic potential of this regulatory pathway (WO2019/036509A1, incorporated herein by reference). Further highlighting its pharmacologic potential, DMI has recently been reported to 1) demonstrate a protective effect on cerebral ischemia/reperfusion injury, thereby offering potential for the treatment of ischemic stroke (Zhang D. et al., 2019); 2) provide protection from the cardiotoxic effects of doxorubicin (Shan Q. et al., 2019); and 3) protect against lippolysacchride-induced mastitis in mice by activating MAPKs and NRFrf2 while inhibiting NF-κB signaling pathways (Zhao C. et al., 2019). Furthermore, DMI is said to have utility in preventing and treating ulcerative colitis and canceration thereof (CN110731955, Sun Yat-sen University Cancer Center); and has been reported to protect against fungal keratitis by activating the NRF2/HO-1 signalling pathway (Gu L. et al., 2020). Nevertheless, it should be noted that DMI is not metabolised to itaconic acid intracellularly (ElAzzouny M. et al., 2017). Other α,β-unsaturated esters exhibit IL-1β-lowering effects in macrophages by inhibiting the NLRP3 inflammasome (Cocco M. et al., 2017 and 2014), and have been demonstrated to inhibit the TLR4 pathway, leading ultimately to suppression of LPS-induced stimulation of NF-κB, tumour necrosis factor (TNF)-α, IL-1β and nitric oxide release (Zhang S. et al., 2012).

Other itaconic acid derivatives have been demonstrated to elicit anti-inflammatory effects (Bagavant G. et al., 1994). A notable example is 4-octyl itaconic acid (401), an itaconate derivative with improved cellular uptake. Since the α,β-unsaturated carboxylic acid is not esterified in 401, this electrophile exhibits low reactivity with biological thiols (Schmidt T. J. et al., 2007), much like the situation encountered with itaconic acid itself. As a result of its low reactivity/electrophilicity, the NRF2-activating effects of 401 are not attenuated by GSH, in contrast to the findings with the much more reactive DMI. In this latter case, the α,β-unsaturated carboxylic acid is esterified and, as a consequence, the IL-6-lowering and NRF2-activating effects of DMI are reversed by the thiols N-acetylcysteine and GSH, respectively. Through the reaction with KEAP1 and the resulting NRF2 activation, as well as GAPDH inhibition (Liao S.-T. et al., 2019), 401 has been demonstrated to produce a wide range of interesting biological effects, including: 1) protection of neuronal cells from hydrogen peroxide (Liu H. et al., 2018); 2) inhibition of proinflammatory cytokine production in peripheral blood mononuclear cells of SLE patients (Tang C. et al., 2018); and 3) protection of human umbilical vein endothelial cells from high glucose (Tang C. et al., 2019); 4) inhibition of osteoclastogenesis by suppressing the E3 ubiquitin ligase Hrd1 and activating NRF2 signaling (Sun X. et al., 2019); 5) induction of repression of STING by NRF2 and type I IFN production in cells from patients with STING-dependent interferonopathies (Olagnier D. et al., 2018); 6) protection against renal fibrosis via inhibiting the TGF-beta/Smad pathway, autophagy and reducing generation of reactive oxygen species (Tian F. et al., 2020); 7) reduction of brain viral burden in mice intracranially injected with Zika virus (Daniels B. P. et al. 2019); and 8) protection against liver ischemia-reperfusion injury (Yi F. et al. 2020). Furthermore, itaconate has been reported to modulate tricarboxylic acid and redox metabolism to mitigate reperfusion injury (Cordes T. et al., 2020). In addition, raised plasma itaconate levels demonstrate a clear correlation with reduction in rheumatoid arthritis disease activity scores following commencement of therapy with conventional disease modifying anti-rheumatic drug (cDMARD) therapy (Daly R. et al. 2019).

Artyomov et al. (WO2017/142855; WO2019/036509) disclose the use of itaconate, malonate or a derivative thereof as an immunomodulatory agent. WO2020/222011 and WO2020/222010 (Sitryx Therapeutics) disclose certain itaconate derivatives.

Zhang et al. (Chemoproteomic profiling of itaconations in Salmonella, Chem. Sci. 2021, 12, 6059) discloses itaconate-based bioorthogonal probes, which are said to enable quantitative and site-specific profiling of itaconated proteins and sites in Salmonella.

In spite of the above findings, there remains a need to identify and develop new therapeutics possessing enhanced properties compared to currently marketed or other known anti-inflammatory agents. The present inventors have now discovered novel compounds which are effective at reducing cytokine release in cells and/or in activating NRF2-driven effects. These properties, amongst others, make them potentially useful in treating inflammatory disease or a disease associated with an undesirable immune response.

### SUMMARY OF THE INVENTION

The present invention provides a compound of formula (I):

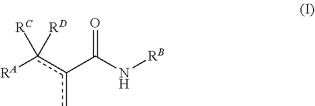

wherein,
R^{A} is:

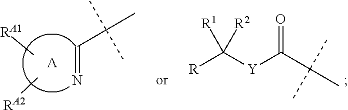

wherein when R^{A} is

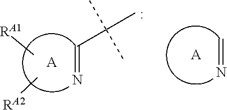

represents a 5 membered heteroaryl ring, which in addition to the C═N shown contains one or more further heteroatoms independently selected from N, O and S; or

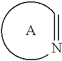

represents a 6 membered heteroaryl ring, which in addition to the C═N shown optionally contains one or more further N atoms;
R^{A1} is selected from the group consisting of C₁₋₁₀ alkyl, C₂₋₁₀ alkenyl, C₂₋₁₀ alkynyl, —(CH₂)₀₋₆—C₃₋₁₀-cycloalkyl, —(CH₂)₀₋₆—C₅₋₁₀ spirocycloalkyl, —(CH₂)₀₋₆-aryl and O-aryl;
   wherein R^{A1} is optionally substituted by one or more R^{A1} wherein each R^{A1} is independently selected from the group consisting of halo, C₁₋₆ alkyl, C₁₋₆ haloalkyl, hydroxy, cyano, OG¹, S(O)₀₋₂G¹, SF₅, (CH₂)₀₋₃C₃₋₇ cycloalkyl and 5-7-membered heterocyclyl wherein said C₃₋₇ cycloalkyl and said 5-7-membered heterocyclyl are optionally substituted by one or more R^{A″} wherein each R^{A″} is independently selected from the group consisting of halo, C₁₋₃ alkyl and C₁₋₃ haloalkyl; wherein two alkyl groups which are attached to the same carbon atom are optionally joined to form a C₃₋₇ cycloalkyl ring; wherein the C₃₋₁₀ cycloalkyl group is optionally fused to a phenyl ring which phenyl ring is optionally substituted by one or more halo atoms; and/or R^{A1} is optionally substituted by one phenyl ring which is optionally substituted by C₁₋₂ haloalkyl, C₁₋₂ haloalkoxy or one or more halo atoms;
   wherein, when two alkyl R^{A′} substituents are attached to a single carbon atom of R^{A′}, the two R^{A′} substituents may combine to form a 3- to 7-membered cycloalkyl ring;
   wherein G¹ is C₁₋₆ alkyl, C₃₋₇ cycloalkyl, C₁₋₆ haloalkyl, or (CH₂)₀₋₁phenyl wherein G¹ is optionally substituted by one or more G^{1′} wherein G^{1′} is independently selected from the group consisting of halo, C₁₋₂ alkyl, C₁₋₂ haloalkyl, hydroxy, cyano, nitro, C₁₋₂ alkoxy and C₁₋₂ haloalkoxy;
R^{A2} is selected from the group consisting of halo, C₁₋₆ alkyl, C₂₋₆ alkenyl, C₂₋₆ alkynyl, C₁₋₆ haloalkyl, hydroxy, cyano, nitro, NR³R⁴, OG² and S(O)₀₋₂G²;
   wherein G² is C₁₋₆ alkyl, C₃₋₇ cycloalkyl, C₁₋₆ haloalkyl, or phenyl which is optionally substituted by one or more G^{2′} wherein each G^{2′} is independently selected from the group consisting of halo, C₁₋₂ alkyl, C₁₋₂ haloalkyl, hydroxy, cyano, nitro, C₁₋₂ alkoxy and C₁₋₂ haloalkoxy; and
   wherein R³ and R⁴ are independently H or C₁₋₂ alkyl or, taken together, R³ and R⁴ may combine to form a 5-7-membered heterocyclic ring;
or R^{A2} is absent;
   wherein when R^{A} is

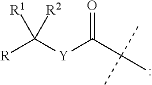

Y is O or NH; and
R is C₁₋₁₀ alkyl, and R¹ and R² are independently selected from the group consisting of H, C₁₋₄ alkyl and C₁₋₄ haloalkyl or R¹ and R² join to form a C₃₋₄ cycloalkyl ring; wherein R is optionally substituted by one or more R^{a} wherein each R^{a} is independently selected from the group consisting of halo, C₁₋₂ haloalkyl and C₁₋₂ haloalkoxy; or
R is selected from the group consisting of C₃₋₁₀ cycloalkyl, phenyl and 5- or 6-membered heteroaryl, and R¹ and R² are independently selected from the group consisting of H, C₁₋₄ alkyl and C₁₋₄ haloalkyl, or R¹ and R² join to form a C₃₋₄ cycloalkyl ring or a 4- to 6-membered heterocyclic ring, wherein the C₃₋₄ cycloalkyl ring or 4- to 6-membered heterocyclic ring is optionally substituted by methyl, halo or cyano; wherein R is optionally substituted by one or more R^{b} wherein each R^{b} is independently selected from the group consisting of halo, C₁₋₄ alkyl, C₁₋₄ haloalkyl, C₁₋₄ alkoxy, C₁₋₄ haloalkoxy and cyano; or
R is H, methyl or CF₃ and R¹ and R² are joined to form a C₃₋₁₀ cycloalkyl ring, wherein the C₃₋₁₀ cycloalkyl ring is optionally substituted by one or more R^{e} wherein each R^{e} is independently selected from the group consisting of halo, C₁₋₂ alkyl, C₁₋₂ haloalkyl, C₁₋₂ alkoxy and C₁₋₂ haloalkoxy, and/or wherein the C₃₋₁₀ cycloalkyl ring is optionally substituted by two R^{e} groups wherein the two R^{e} groups are attached to the same carbon atom and are joined to form a C₁₋₄ cycloalkyl ring;
R^{B} is heteroaryl wherein the heteroaryl is optionally substituted by one or more R^{B′} wherein each
R^{B′} is independently selected from the group consisting of halo, C₁₋₂ alkyl, C₁₋₂ haloalkyl, C₁₋₂ alkoxy, C₁₋₂ haloalkoxy, CO₂H, CO₂C₁₋₄ alkyl and NR⁵R⁶, wherein R⁵ and R⁶ are independently H, C₁₋₄ alkyl and C₃₋₆ cycloalkyl, or R⁵ and R⁶ join to form a 4-7-membered heterocyclic ring; or
R^{B} is a 4- to 6-membered heterocyclic ring comprising one or two ring heteroatoms selected from N, O and S and optionally substituted on a ring nitrogen atom with C₁₋₂ alkyl and/or on a ring sulfur atom with one or two oxo substituents; or
R^{B} is selected from H, C₁₋₂ alkyl and C₁₋₂ haloalkyl, where alkyl and haloalkyl groups are optionally substituted with a substituent R^{B″}.
wherein R^{B″} is selected from C(O)OH, C(O)O(C₁₋₂ alkyl), SO₂(C₁₋₂ alkyl), and a 5- or 6-membered heterocyclic ring comprising one or two ring heteroatoms selected from N, O and S and optionally substituted with C₁₋₂ alkyl; and
R^{C} and R^{D} are each independently H, C₁₋₂ alkyl, hydroxy, fluoro or C₁₋₂ alkoxy; or R^{C} and R^{D} may join to form a C₃₋₅ cycloalkyl ring;
wherein

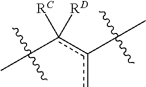

in the compound of formula (I) represents:

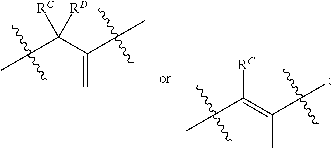

and wherein
   the total number of carbon atoms in groups R^{A1} and R^{A2} taken together including their optional substituents is 1 to 14; and
   wherein the total number of carbon atoms in groups R, R¹ and R² taken together, including their optional substituents, and including the carbon to which R, R¹ and R² are attached, is 3 to 14;
or a pharmaceutically acceptable salt and/or solvate thereof.

The present invention provides a pharmaceutical composition comprising a compound of formula (I) or a pharmaceutically acceptable salt and/or solvate thereof.

The present invention provides a compound of formula (I) or a pharmaceutically acceptable salt and/or solvate thereof for use as a medicament.

The present invention provides a compound of formula (I) or a pharmaceutically acceptable salt and/or solvate thereof for use in treating or preventing an inflammatory disease or a disease associated with an undesirable immune response.

The present invention provides the use of a compound of formula (I) or a pharmaceutically acceptable salt and/or solvate thereof in the manufacture of a medicament for treating or preventing an inflammatory disease or a disease associated with an undesirable immune response.

The present invention provides a method of treating or preventing an inflammatory disease or a disease associated with an undesirable immune response, which comprises administering a compound of formula (I) or a pharmaceutically acceptable salt and/or solvate thereof.

Also provided are intermediate compounds of use in the preparation of compounds of formula (I).

### DETAILED DESCRIPTION OF THE INVENTION

### Compounds of Formula (I)

Embodiments and preferences set out herein with respect to the compound of formula (I) apply equally to the pharmaceutical composition, compound for use, use and method aspects of the invention.

As used herein, the term “alkyl”, such as “C₁₋₁₀ alkyl”, “C₁₋₆ alkyl”, “C₁₋₄ alkyl”, “C₁₋₃ alkyl” or “C₁₋₂ alkyl”, refers to a straight or branched fully saturated hydrocarbon group having the specified number of carbon atoms. The term encompasses, without limitation, methyl, ethyl, n-propyl, iso-propyl, n-butyl, iso-butyl, sec-butyl, tert-butyl, n-pentyl, n-hexyl, n-heptyl, n-octyl, n-nonyl and n-decyl. Branched variants are also included such as (n-Bu)₂CH—, n-pentyl-CH(CH₂CH₃)—, n-pentyl-C(CH₃)₂—, n-hexyl-C(CH₃)₂— and n-heptyl-CH(CH₃)—. The term “alkyl” also encompasses “alkylene” which is a bifunctional straight or branched fully saturated hydrocarbon group having the stated number of carbon atoms. Example “alkylene” groups include, without limitation, methylene, ethylene, n-propylene, n-butylene, n-pentylene, n-heptylene, n-hexylene and n-octylene.

The term “alkoxy” refers to an alkyl group, such as “C₁₋₁₀ alkyl”, “C₁₋₆ alkyl”, “C₁₋₄ alkyl”, “C₁₋₃ alkyl” or “C₁₋₂ alkyl”, as defined above, singularly bonded via an oxygen atom. Examples of haloalkoxy groups include, without limitation, OCH₃.

The term “cycloalkyl”, such as “C₃₋₁₀ cycloalkyl”, “C₃₋₇ cycloalkyl” or “C₃₋₄ cycloalkyl”, refers to a fully saturated cyclic hydrocarbon group having the specified number of carbon atoms. The term encompasses, without limitation, cyclopropyl, cyclobutyl, cyclopentyl, cyclohexyl, cycloheptyl, cyclooctyl, cyclononyl and cyclodecyl as well as bridged systems such as bicyclo[1.1.1]pentyl, bicyclo[2.2.1]heptyl, bicyclo[2.2.2]octyl and adamantyl.

The term “haloalkyl”, such as “C₁₋₆ haloalkyl”, “C₁₋₄ haloalkyl”, “C₁₋₃ haloalkyl”, “C₁₋₂ haloalkyl” or “C₁ haloalkyl”, refers to a straight or a branched fully saturated hydrocarbon chain containing the specified number of carbon atoms and at least one halogen atom, such as fluoro or chloro, especially fluoro. An example of haloalkyl is CF₃. Further examples of haloalkyl are CHF₂, CF₂CH₃ and CH₂CF₃. These examples of haloalkyl groups are not intended to be limiting.

The term “haloalkoxy” refers to a haloalkyl group, such as “C₁₋₆ haloalkyl”, “C₁₋₄ haloalkyl”, “C₁₋₃ haloalkyl”, “C₁₋₂ haloalkyl” or “C₁ haloalkyl”, as defined above, singularly bonded via an oxygen atom. Examples of haloalkoxy groups include, without limitation, OCF₃, OCHF₂ and OCH₂CF₃.

The term “halo” refers to fluorine, chlorine, bromine or iodine. Particular examples of halo are fluorine, chlorine and bromine, especially fluorine.

The term “4-7-membered heterocyclic ring” such as “5-7-membered heterocyclic ring” refers to a non-aromatic cyclic group having the stated number of ring atoms and wherein at least one of the ring atoms is a heteroatom selected from N, O, S and B. The term “heterocyclic ring” is interchangeable with “heterocyclyl”. The term encompasses, without limitation, azetidinyl, oxetanyl, thietanyl, pyrrolidinyl, tetrahydrofuranyl, tetrahydrothienyl, tetrahydropyranyl, piperidinyl, piperazinyl, morpholinyl, thiomorpholinyl, azepanyl, oxepanyl and thiepanyl. 4-7-membered heterocyclyl groups can typically be substituted by one or more (e.g. one or two) oxo groups. Suitably, a sulphur atom is substituted by one or two oxo groups thus forming S═O or SO₂.

The term “aryl” refers to a cyclic group with 6 to 10 carbon atoms, the cyclic group being aromatic. The term encompasses naphthyl and phenyl, and is suitably phenyl.

The term “heteroaryl” refers to a cyclic group with aromatic character wherein at least one of the atoms in the cyclic group is a heteroatom independently selected from N, O and S. The term encompasses, without limitation, pyrrolyl, furanyl, thienyl, imidazolyl, pyrazolyl, thiazolyl, oxadiazolyl, thiadiazolyl, triazolyl, oxazolyl, tetrazolyl, pyridyl, pyrimidinyl, pyradizinyl and pyrazinyl.

The term “oxo” refers to an oxygen atom linked via a double bond to the remainder of the molecule. The double bond may be attached at its other end to a carbon, nitrogen or sulfur atom.

The term “C₅₋₁₀ spirocycloalkyl” refers to a cycloalkyl group comprising two cycloalkyl rings joined by a spiro linkage such that the two rings are linked at a single atom and having a total of from 5 to 10 ring atoms. The spirocycloalkyl group is joined to the remainder of the molecule via an atom other than the one which links the two rings. Examples include, without limitation, spiro [4.3]octane and spiro [5.4] decane.

Where substituents are indicated as being optionally substituted in formula (I) in the embodiments and preferences set out below, the optional substituent may be attached to an available carbon atom, which means a carbon atom which is attached to a hydrogen atom i.e. a C—H group. The optional substituent replaces the hydrogen atom attached to the carbon atom.

In one embodiment, the compound of formula (I) is a compound of formula (IA):

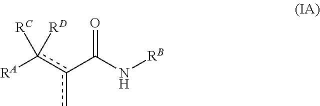

wherein
R^{A} is

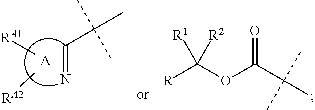

when R^{A} is

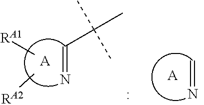

represents a 5 membered heteroaryl ring, which in addition to the C═N shown contains one or more further heteroatoms independently selected from N, O and S; or

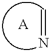

represents a 6 membered heteroaryl ring, which in addition to the C═N shown optionally contains one or more further N atoms;
R^{A1} is selected from the group consisting of C₁₋₁₀ alkyl, C₂₋₁₀ alkenyl, C₂₋₁₀ alkynyl, —(CH₂)₀₋₆—C₃₋₁₀-cycloalkyl, —(CH₂)₀₋₆—C₅₋₁₀ spirocycloalkyl, —(CH₂)₀₋₆-aryl and O-aryl;
   wherein R^{A1} is optionally substituted by one or more R^{A′} wherein R^{A′} is independently selected from the group consisting of halo, C₁₋₆ alkyl, C₁₋₆ haloalkyl, hydroxy, cyano, OG¹, S(O)₀₋₂G¹, SF₅, (CH₂)₀₋₃C₃₋₇ cycloalkyl and 5-7-membered heterocyclyl wherein said C₃₋₇ cycloalkyl and said 5-7-membered heterocyclyl are optionally substituted by one or more R^{A″} wherein R^{A″} is independently selected from the group consisting of halo, C₁₋₃ alkyl and C₁₋₃ haloalkyl; wherein two alkyl groups which are attached to the same carbon atom are optionally joined to form a C₃₋₇ cycloalkyl ring; wherein the C₃₋₁₀ cycloalkyl group is optionally fused to a phenyl ring which phenyl ring is optionally substituted by one or more halo atoms; and/or R^{A1} is optionally substituted by one phenyl ring which is optionally substituted by C₁₋₂ haloalkyl, C₁₋₂ haloalkoxy or one or more halo atoms;
   wherein G¹ is C₁₋₆ alkyl, C₃₋₇ cycloalkyl, C₁₋₆ haloalkyl, or (CH₂)₀₋₁phenyl wherein G¹ is optionally substituted by one or more G^{1′} wherein G^{1′} is independently selected from the group consisting of halo, C₁₋₂ alkyl, C₁₋₂ haloalkyl, hydroxy, cyano, nitro, C₁₋₂ alkoxy and C₁₋₂ haloalkoxy;
R^{A2} is selected from the group consisting of halo, C₁₋₆ alkyl, C₂₋₆ alkenyl, C₂₋₆ alkynyl, C₁₋₆ haloalkyl, hydroxy, cyano, nitro, NR³R⁴, OG² and S(O)₀₋₂G²;
   wherein G² is C₁₋₆ alkyl, C₃₋₇ cycloalkyl, C₁₋₆ haloalkyl, or phenyl which is optionally substituted by one or more G^{2′} wherein G^{2′} is independently selected from the group consisting of halo, C₁₋₂ alkyl, C₁₋₂ haloalkyl, hydroxy, cyano, nitro, C₁₋₂ alkoxy and C₁₋₂ haloalkoxy; and
   wherein R³ and R⁴ are independently H or C₁₋₂ alkyl or, taken together, R³ and R⁴ may combine to form a 5-7-membered heterocyclic ring;
or R^{A2} is absent;
   wherein when R^{A} is

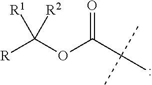

R is C₁₋₁₀ alkyl, and R¹ and R² are independently selected from the group consisting of H, C₁₋₄ alkyl and C₁₋₄ haloalkyl or R¹ and R² join to form a C₃₋₄ cycloalkyl ring; wherein R is optionally substituted by one or more R^{a} wherein R^{a} is independently selected from the group consisting of halo, C₁₋₂ haloalkyl and C₁₋₂ haloalkoxy; or
R is selected from the group consisting of C₃₋₁₀ cycloalkyl, phenyl and 5- or 6-membered heteroaryl, and R¹ and R² are independently selected from the group consisting of H, C₁₋₄ alkyl and C₁₋₄ haloalkyl, or R¹ and R² join to form a C₃₋₄ cycloalkyl ring or a C₄₋₆ heterocycloalkyl ring, wherein the C₃₋₄ cycloalkyl ring is optionally substituted by methyl, halo or cyano; wherein R is optionally substituted by one or more R^{b} wherein R^{b} is independently selected from the group consisting of halo, C₁₋₄ alkyl, C₁₋₄ haloalkyl, C₁₋₄ alkoxy, C₁₋₄ haloalkoxy and cyano; or
R is H, methyl or CF₃ and R¹ and R² are joined to form a C₃₋₁₀ cycloalkyl ring, wherein the C₃₋₁₀ cycloalkyl ring is optionally substituted by one or more R^{e} wherein R^{e} is independently selected from the group consisting of halo, C₁₋₂ alkyl, C₁₋₂ haloalkyl, C₁₋₂ alkoxy and C₁₋₂ haloalkoxy, and/or wherein the C₃₋₁₀ cycloalkyl ring is optionally substituted by two R^{e} groups wherein the two R^{e} groups are attached to the same carbon atom and are joined to form a C₄₋₆ cycloalkyl ring;
R^{B} is heteroaryl wherein the heteroaryl is optionally substituted by one or more R^{B′} wherein R^{B′} is independently selected from the group consisting of halo, C₁₋₂ alkyl, C₁₋₂ haloalkyl, C₁₋₂ alkoxy, C₁₋₂ haloalkoxy, CO₂H, CO₂C₁₋₄ alkyl and NR⁵R⁶, wherein R⁵ and R⁶ are independently H, C₁₋₄ alkyl and C₃₋₆ cycloalkyl, or R⁵ and R⁶ join to form a 4-7-membered heterocyclic ring; and
R^{C} and R^{D} are each independently H, C₁₋₂ alkyl, hydroxy, fluoro or C₁₋₂ alkoxy; or R^{C} and R^{D} may join to form a C₃₋₅ cycloalkyl ring;
wherein

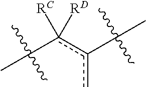

in the compound of formula (I) represents:

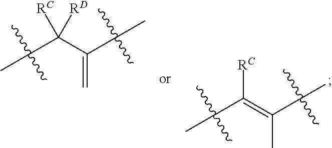

and wherein
   the total number of carbon atoms in groups R^{A} and R^{A2} taken together including their optional substituents is 1 to 14; and
   wherein the total number of carbon atoms in groups R, R¹ and R² taken together, including their optional substituents, and including the carbon to which R, R¹ and R² are attached, is 3 to 14;
or a pharmaceutically acceptable salt and/or solvate thereof.

In one embodiment, R^{A} is:

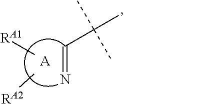

wherein:

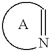

represents a 5 membered heteroaryl ring, which in addition to the C═N shown contains one or more further heteroatoms independently selected from N, O and S; or

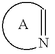

represents a 6 membered heteroaryl ring, which in addition to the C═N shown optionally contains one or more further N atoms;
R^{A1} is selected from the group consisting of C₁₋₁₀ alkyl, C₂₋₁₀ alkenyl, C₂₋₁₀ alkynyl, —(CH₂)₀₋₆—C₃₋₁₀-cycloalkyl, —(CH₂)₀₋₆—C₅₋₁₀ spirocycloalkyl, —(CH₂)₀₋₆-aryl and O-aryl;
   wherein R^{A1} is optionally substituted by one or more R^{A′} wherein each R^{A′} is independently selected from the group consisting of halo, C₁₋₆ alkyl, C₁₋₆ haloalkyl, hydroxy, cyano, OG¹, S(O)₀₋₂G¹, SF₅, (CH₂)₀₋₃C₃₋₇ cycloalkyl and 5-7-membered heterocyclyl wherein said C₃₋₇ cycloalkyl and said 5-7-membered heterocyclyl are optionally substituted by one or more
   R^{A″} wherein R^{A″} is independently selected from the group consisting of halo, C₁₋₃ alkyl and C₁₋₃ haloalkyl; wherein two alkyl groups which are attached to the same carbon atom are optionally joined to form a C₃₋₇ cycloalkyl ring; wherein the C₃₋₁₀ cycloalkyl group is optionally fused to a phenyl ring which phenyl ring is optionally substituted by one or more halo atoms; and/or R^{A1} is optionally substituted by one phenyl ring which is optionally substituted by C₁₋₂ haloalkyl, C₁₋₂ haloalkoxy or one or more halo atoms;
   wherein when two alkyl R^{A′} substituents are attached to a single carbon atom of R^{A1}, the two R^{A} substituents may combine to form a 3- to 7-membered cycloalkyl ring wherein G¹ is C₁₋₆ alkyl, C₃₋₇ cycloalkyl, C₁₋₆ haloalkyl, or (CH₂)₀₋₁phenyl wherein G¹ is optionally substituted by one or more G^{1′} wherein each G^{1′} is independently selected from the group consisting of halo, C₁₋₂ alkyl, C₁₋₂ haloalkyl, hydroxy, cyano, nitro, C₁₋₂ alkoxy and C₁₋₂ haloalkoxy;
R^{A2} is selected from the group consisting of halo, C₁₋₆ alkyl, C₂₋₆ alkenyl, C₂₋₆ alkynyl, C₁₋₆ haloalkyl, hydroxy, cyano, nitro, NR³R⁴, OG² and S(O)₀₋₂G²;
   wherein G² is C₁₋₆ alkyl, C₃₋₇ cycloalkyl, C₁₋₆ haloalkyl, or phenyl which is optionally substituted by one or more G^{2′} wherein each G^{2′} is independently selected from the group consisting of halo, C₁₋₂ alkyl, C₁₋₂ haloalkyl, hydroxy, cyano, nitro, C₁₋₂ alkoxy and C₁₋₂ haloalkoxy; and
   wherein R³ and R⁴ are independently H or C₁₋₂ alkyl or, taken together, R³ and R⁴ may combine to form a 5-7-membered heterocyclic ring;
or R^{A2} is absent.

The group

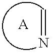

may also be written as

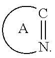

In one embodiment,

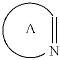

represents a 5 membered heteroaryl ring, which in addition to the C═N shown contains one or more (e.g., one or two) further heteroatoms independently selected from N, O and S.

In one embodiment,

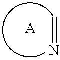

represents a 5 membered heteroaryl ring selected from the group consisting of imidazole, pyrazole, oxazole, isoxazole, thiazole, isothiazole, 1,2,3-triazole, 1,2,4-triazole, 1,2,4-oxadiazole, 1,2,5-oxadiazole, 1,3,4-oxadiazole, 1,2,4-thiadiazole, 1,2,5-thiadiazole, 1,3,4-thiadiazole and tetrazole.

When

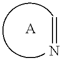

represents imidazole, it is intended to represent

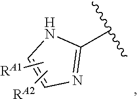

in formula (I). For the avoidance of doubt, substituent R^{A1} and/or R^{A2} (if present) can be bound to a carbon or nitrogen atom of the imidazole moiety.

When

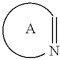

represents pyrazole, it is intended to represent

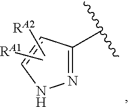

in formula (I). For the avoidance of doubt, substituent R^{A1} and/or R^{A2} (if present) can be bound to a carbon or nitrogen atom of the pyrazole moiety.

When

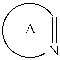

represents oxazole, it is intended to represent

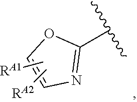

in formula (I).

When

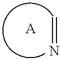

represents isoxazole, it is intended to represent

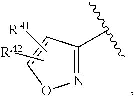

in formula (I).

When

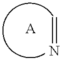

represents thiazole, it is intended to represent

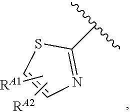

in formula (I).

When

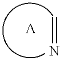

represents isothiazole, it is intended to represent

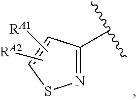

in formula (I).

When

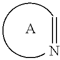

represents 1,2,3-triazole, it is intended to represent

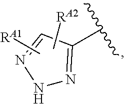

in formula (I). For the avoidance of doubt, substituent R^{A1} and/or R^{A2} (if present) can be bound to a carbon or nitrogen atom of the 1,2,3-triazole moiety.

When

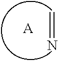

represents 1,2,4-triazole, it is intended to represent

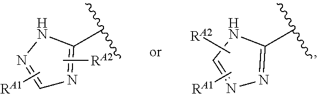

in formula (I). For the avoidance of doubt, substituent R^{A1} and/or R^{A2} (if present) can be bound to a carbon or nitrogen atom of the 1,2,4-triazole moiety.

When

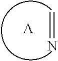

represents 1,2,4-oxadiazole, it is intended to represent

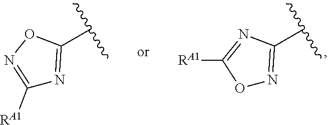

in formula (I).

When

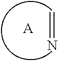

represents 1,2,5-oxadiazole, it is intended to represent

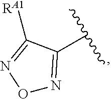

in formula (I).

When

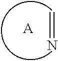

represents 1,3,4-oxadiazole, it is intended to represent

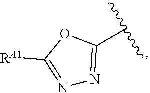

in formula (I).

When

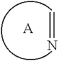

represents 1,2,4-thiadiazole, it is intended to represent

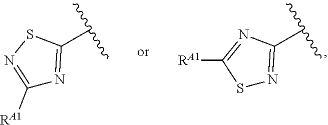

in formula (I).

When

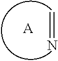

represents 1,2,5-thiadiazole, it is intended to represent

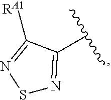

in formula (I).

When

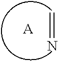

represents 1,3,4-thiadiazole, it is intended to represent

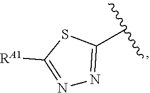

in formula (I).

When

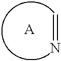

represents tetrazole, it is intended to represent

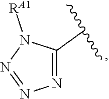

in formula (I).

In one embodiment,

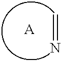

represents an oxadiazole, in particular 1,2,4-oxadiazole.

Suitably, the 1,2,4-oxadiazole is

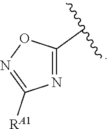

In one embodiment,

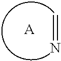

represents a 6 membered heteroaryl ring, which in addition to the C═N shown optionally contains one or more (e.g., one or two) further N atoms.

In one embodiment,

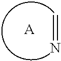

represents a 6 membered heteroaryl ring selected from the group consisting of pyridine, pyridazine, pyrimidine, pyrazine and triazine.

When

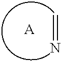

represents pyridine, it is intended to represent

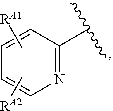

in formula (I).

When

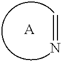

represents pyridazine, it is intended to represent

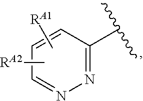

in formula (I).

When

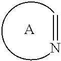

represents pyrimidine, it is intended to represent

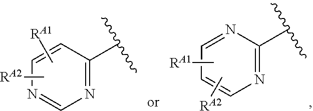

in formula (I).

When

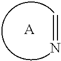

represents pyrazine, it is intended to represent

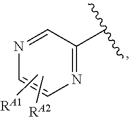

in formula (I).

When

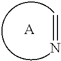

represents triazine, it is intended to represent

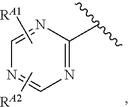

in formula (I).

In the representations above, where a substituent is not indicated as being bound to a carbon atom or nitrogen atom and is instead shown as intersecting a double or single bond of a heteroaryl compound, this indicates that the point of attachment is undefined, and may be any attachment point which is chemically feasible. Furthermore, each of the above mentioned heteroaryl groups is shown as a single tautomer. The skilled person recognises that although a single tautomer is shown, the compound may exist as a mixture of tautomeric forms. Thus, the invention extends to all tautomeric forms of the compounds of formula (I).

R^{A1} is selected from the group consisting of C₁₋₁₀ alkyl, C₂₋₁₀ alkenyl, C₂₋₁₀ alkynyl, —(CH₂)₀₋₆—C₃₋₁₀-cycloalkyl, —(CH₂)₀₋₆—C₅₋₁₀ spirocycloalkyl, —(CH₂)₀₋₆-aryl and O-aryl.

In one embodiment, R^{A1} is —(CH₂)₀₋₆—C₃₋₁₀ cycloalkyl, wherein said C₃₋₁₀ cycloalkyl group is optionally fused to a phenyl ring which phenyl ring is optionally substituted by one or more (such as one, two or three e.g. one) halo atoms (e.g. fluoro).

Suitably, R^{A1} is —(CH₂)₀₋₆-aryl such as CH₂aryl wherein aryl is phenyl.

In one embodiment, R^{A1} is not substituted. In another embodiment, R^{A1} is substituted by one or more (such as one, two or three, e.g. one) R^{A′}.

R^{A′} is independently selected from the group consisting of halo, C₁₋₆ alkyl, C₁₋₆ haloalkyl, hydroxy, cyano, OG¹, S(O)₀₋₂G¹, SF₅, (CH₂)₀₋₃C₃₋₇ cycloalkyl and 5-7-membered heterocyclyl. In one embodiment, said C₃₋₇ cycloalkyl and said 5-7-membered heterocyclyl are not substituted. In another embodiment, said C₃₋₇ cycloalkyl and said 5-7-membered heterocyclyl are substituted by one or more (such as one, two or three e.g. one) R^{A″}, wherein R^{A″} is defined above.

In one embodiment, two alkyl groups R^{A′} which are attached to the same carbon atom are optionally joined (e.g. are joined) to form a C₃₋₇ cycloalkyl ring. For example, when R^{A1} is CH₂aryl, suitably two methyl R^{A′} groups may be attached to the CH₂ carbon of CH₂aryl and are joined to form a cyclopropyl ring.

In one embodiment, R^{A1} is optionally substituted by one phenyl ring which is optionally substituted by C₁₋₂ haloalkyl, C₁₋₂ haloalkoxy or one or more (such as one, two or three e.g. one) halo atoms.

In one embodiment, R^{A′} is C₁₋₈ alkyl such as C₄ alkyl. In a second embodiment, R^{A′} is S(O)₀₋₂G¹ such as S(O)₀-G¹ i.e. SG¹, wherein G¹ is defined above.

In one embodiment, G¹ is not substituted. In another embodiment, G¹ is substituted by one or more (such as one, two or three, e.g. one) G^{1′} wherein G^{1′} is defined above.

Suitably, G¹ is C₁₋₆ haloalkyl such as CF₃.

In one embodiment, R^{A1} is substituted by two alkyl groups which are attached to the same carbon atom are optionally joined to form a C₃₋₇ cycloalkyl ring (such as a cyclopropyl ring) and R^{A1} is additionally substituted by one or more R^{A′}, wherein R^{A′} is as defined above.

Suitably, R^{A2} is absent.

In another embodiment, R^{A} is:

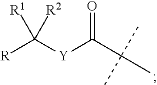

wherein Y, R, R¹ and R² are as defined above.

In one embodiment, Y is O.

In an alternative embodiment, Y is NH.

In one embodiment, R is C₁₋₁₀ alkyl, and R¹ and R² are independently selected from the group consisting of H, C₁₋₄ alkyl and C₁₋₄ haloalkyl or R¹ and R² join to form a C₃₋₄ cycloalkyl ring.

Suitably, R is C₆₋₁₀ alkyl such as C alkyl.

In one embodiment, R¹ and R² are independently selected from the group consisting of H, C₁₋₄ alkyl and C₁₋₄ haloalkyl. In another embodiment, R¹ and R² join to form a C₃₋₄ cycloalkyl ring.

Suitably, R₁ and R₂ are independently C₁₋₄ alkyl such as methyl.

In one embodiment, R is not substituted. In another embodiment, R is substituted by one or more (such as one, two, or three, e.g. one) R^{a} wherein R^{a} is defined above.

In another embodiment, R is selected from the group consisting of C₃₋₁₀ cycloalkyl, phenyl and 5- or 6-membered heteroaryl, and R¹ and R² are independently selected from the group consisting of H, C₁₋₄ alkyl and C₁₋₄ haloalkyl, or R¹ and R² join to form a C₃₋₄ cycloalkyl ring or a C₄₋₆ heterocyclic ring, wherein the C₃₋₄ cycloalkyl ring is optionally substituted by methyl, halo or cyano.

In one embodiment, R is C₃₋₁₀ cycloalkyl. In a second embodiment, R is phenyl. In a third embodiment, R is 5- or 6-membered heteroaryl, such as pyridyl, for example pyridin-2-yl.

In one embodiment, R¹ and R² are independently selected from the group consisting of H, C₁₋₄ alkyl and C₁₋₄ haloalkyl, especially H or methyl. In some suitable compounds, R¹ and R² are both H. In other suitable compounds R¹ is and R² are both methyl. In still other suitable compounds, R¹ is H and R² is methyl. In a second embodiment, R¹ and R² join to form a C₃₋₄ cycloalkyl ring. In an embodiment, the C₃₋₄ cycloalkyl ring is not substituted. In another embodiment, the C₃₋₄ cycloalkyl ring is substituted by methyl, halo or cyano. In some particularly suitable compounds, R¹ and R² join to form an unsubstituted cyclobutyl ring. In a third embodiment, R¹ and R² join to form a 4- to 6-membered heterocyclic ring, for example an oxygen-containing heterocyclic ring, such as oxetanyl, which may be substituted by methyl, halo or cyano but is more suitably unsubstituted.

In one embodiment, R is not substituted. In another embodiment, R is substituted by one or more (such as one, two or three e.g. one) R^{b} wherein R^{b} is defined above. Particularly suitable R^{b} substituents include halo, for example fluoro or chloro, and C₁₋₂ haloalkyl, for example trifluoromethyl.

When R is phenyl or pyridyl and is substituted by a single R^{b} substituent, this may be at the 4-position of the phenyl or pyridyl ring with respect to the linkage to —C(R¹)(R²)—. In this case, trifluoromethyl is a particularly suitable R^{b} substituent.

When R is phenyl or pyridyl and is substituted by one or two halo substituents these may be at the 3-position of the phenyl or pyridyl ring with respect to the linkage to —C(R¹)(R²)—. Some compounds of the invention in which R is phenyl or pyridyl have a trifluoromethyl substituent at the para position and a halo substituent at the meta position of the phenyl or pyridyl ring with respect to the linkage to —C(R¹)(R²)—.

Other compounds of the invention in which R is phenyl have two halo substituents at the 3-position of the phenyl ring with respect to the linkage to —C(R¹)(R²)—.

When R is pyridyl, the 3- and 4-positions of the pyridyl ring with respect to the linkage to —C(R¹)(R²)— will not correspond to the 3- and 4-positions of the pyridyl ring as used in the conventional ring numbering system. Examples are shown below.

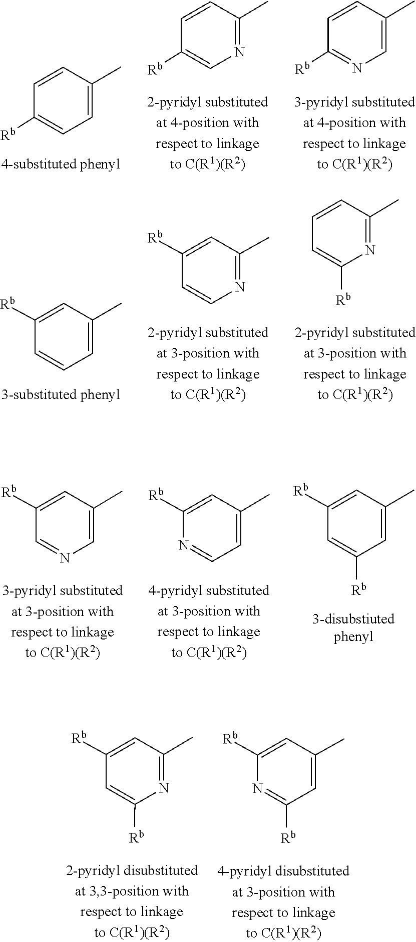

In another embodiment, R is H, methyl or CF₃ and R¹ and R² are joined to form a C₃₋₁₀ cycloalkyl ring.

In one embodiment, R is H. In a second embodiment, R is methyl. In a third embodiment, R is CN.

In one embodiment, the C₃₋₁₀ cycloalkyl ring is not substituted. In another embodiment, the C₃₋₁₀ cycloalkyl ring is substituted by one or more (such as one, two or three e.g. one) R^{e} wherein each R^{e} is independently selected from the group consisting of halo, C₁₋₂ alkyl, C₁₋₂ haloalkyl, C₁₋₂ alkoxy and C₁₋₂ haloalkoxy, and/or wherein the C₃₋₁₀ cycloalkyl ring is optionally substituted by two R^{e} groups wherein the two R^{e} groups are attached to the same carbon atom and are joined to form a C₄₋₆ cycloalkyl ring.

In one embodiment, R^{e} is independently selected from the group consisting of halo, C₁₋₂ alkyl, C₁₋₂ haloalkyl, C₁₋₂ alkoxy and C₁₋₂ haloalkoxy. In a second embodiment, the C₃₋₁₀ cycloalkyl ring is substituted by two R^{e} groups wherein the two R^{e} groups are attached to the same carbon atom and are joined to form a C₄₋₆ cycloalkyl ring.

In any of the above embodiments, and unless otherwise stated, the substituent groups R^{a}, R^{b} and R^{e} may be attached to the same carbon atom, or may be attached to different carbon atoms.

The total number of carbon atoms in groups R, R¹ and R² taken together, including their optional substituents, and including the carbon to which R, R¹ and R² are attached, is 3 to 14. In one embodiment, the total number of carbon atoms is 3 carbon atoms. In another embodiment, the total number of carbon atoms is 4 carbon atoms. In another embodiment, the total number of carbon atoms is 5 carbon atoms. In another embodiment, the total number of carbon atoms is 6 carbon atoms. In another embodiment, the total number of carbon atoms is 7 carbon atoms. In another embodiment, the total number of carbon atoms is 8 carbon atoms. In another embodiment, the total number of carbon atoms is 9 carbon atoms. In another embodiment, the total number of carbon atoms is 10 carbon atoms. In another embodiment, the total number of carbon atoms is 11 carbon atoms. In another embodiment, the total number of carbon atoms is 12 carbon atoms. In another embodiment, the total number of carbon atoms is 13 carbon atoms. In another embodiment, the total number of carbon atoms is 14 carbon atoms.

In some embodiments, R^{B} is heteroaryl wherein the heteroaryl is optionally substituted by one or more R^{B′}. The term “heteroaryl” is defined above. Suitably, the heteroaryl is a 5- or 6-membered heteroaryl ring. Suitably, the heteroaryl is selected from the group consisting of thiazolyl, pyridinyl, pyrimidinyl and pyrazinyl. In one embodiment, the heteroaryl is thiazolyl. In a second embodiment, the heteroaryl is pyridinyl. In a third embodiment, the heteroaryl is pyrazinyl.

In one embodiment, the heteroaryl group R^{B} is not substituted. In another embodiment, R^{B} is substituted by one or more (such as one, two or three e.g. one) R^{B′}.

R^{B′} is independently selected from the group consisting of halo, C₁₋₂ alkyl, C₁₋₂ haloalkyl, C₁₋₂ alkoxy, C₁₋₂ haloalkoxy, CO₂H, CO₂C₁₋₄ alkyl and NR⁵R⁶, wherein R⁵ and R⁶ are independently H, C₁₋₄ alkyl and C₃₋₆ cycloalkyl, or R⁵ and R⁶ join to form a 4-7-membered heterocyclic ring. Suitably, R^{B′} is C₁₋₂ alkyl e.g. methyl.

In alternative embodiments, R^{B} is a 4- to 6-membered heterocyclic ring comprising one or two ring heteroatoms selected from N, O and S and optionally substituted on a ring nitrogen atom with C₁₋₂ alkyl and/or on a ring sulfur atom with one or two oxo substituents.

Suitably, the 4- to 6-membered heterocyclic ring contains at least one nitrogen or sulfur atom and optionally one or more additional ring heteroatoms and are optionally substituted as described above. Examples of suitable heterocyclic rings R^{B} include piperidinyl, N-methyl piperidinyl, pyrrolidinyl, azetidinyl, morpholinyl, thietane-1,1-dioxide and thietane-1-oxide, especially N-methyl piperidinyl and thietane-1,1-dioxide.

In further alternative embodiments, R^{B} is selected from H, C₁₋₂ alkyl and C₁₋₂ haloalkyl, where alkyl and haloalkyl groups are optionally substituted with a substituent R^{B″}, wherein R^{B″} is as defined above.

In some suitable compounds, R^{B} is H.

In other suitable compounds, R^{B} is C₁₋₂ alkyl or C₁₋₂ haloalkyl optionally substituted with a substituent R^{B″} as defined above.

In some compounds, R^{B} is unsubstituted C₁₋₂ alkyl or C₁₋₂ haloalkyl, for example unsubstituted C₁₋₂ alkyl, particularly methyl.

In other compounds, R^{B} is C₁₋₂ alkyl or C₁₋₂ haloalkyl, for example methyl or ethyl, substituted with a substituent R^{B″} as defined above.

Examples of particularly suitable R^{B″} substituents include C(O)OH, SO₂(methyl), and a 5- to 6-membered heterocyclic ring comprising a ring nitrogen atom and optionally one additional ring heteroatom, for example piperidinyl, piperazinyl and morpholinyl, especially morpholinyl such as morpholin-4-yl.

In one embodiment, R^{C} and R^{D} are each independently H, C₁₋₂ alkyl, hydroxy, fluoro or C₁₋₂ alkoxy. In another embodiment, R^{C} and R^{D} may join to form a C₃₋₅ cycloalkyl ring.

Suitably, R^{C} and R^{D} are H.

In one embodiment,

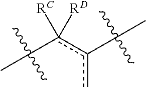

in the compound of formula (I) represents

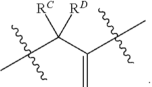

The carbon-carbon double bond in this structure is referred to as “exo”.

In another embodiment,

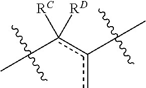

in the compound of formula (I) represents

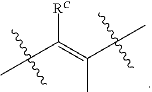

The carbon-carbon double bond in this structure is referred to as “endo”.

In the endo embodiment, the double bond may be cis or trans such that both of the following moieties are covered:

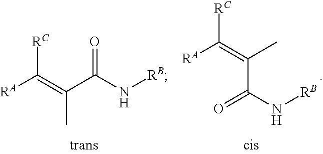

Similarly, as used herein, the following structure:

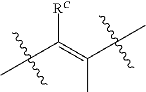

encompasses both cis and trans isomers:

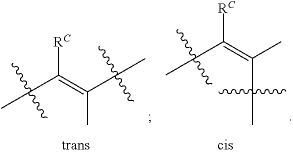

Suitably, the endo double bond in the compound of formula (I) is trans.

The compounds of formula (I) in which the carbon-carbon double bond is endo can generally be obtained by isomerisation from compounds of formula (I) in which the carbon-carbon double bond is exo and such isomerisation may occur in in vitro assays or in vivo following administration of the exo compound. In some cases, isomerisation in in vitro assays, such as in vitro hepatocyte stability assays, or in vivo following administration of the exo compound may be partial and thus lead to a mixture of the endo and exo compound resulting. In some cases, the mixture of endo and exo isomers may contribute to the activity observed in a particular assay. Suitably, compounds of formula (I), such as those in which the carbon-carbon double bond is exo, are stable to isomerisation.

In one embodiment, the molecular weight of the compound of formula (I) is 150 Da-450 Da, suitably 200 Da-400 Da.

In one embodiment there is provided a compound of formula (I), selected from the group consisting of:
N-(5-methylthiazol-2-yl)-2-((3-(1-(4-((trifluoromethyl)thio)phenyl)cyclopropyl)-1,2,4-oxadiazol-5-yl)methyl)acrylamide;
N-(pyrazin-2-yl)-2-((3-(1-(4-((trifluoromethyl)thio)phenyl)cyclopropyl)-1,2,4-oxadiazol-5-yl)methyl)acrylamide;
2-((3-(4-butylbenzyl)-1,2,4-oxadiazol-5-yl)methyl)-N-(pyridin-2-yl)acrylamide;
2-((3-(4-butylbenzyl)-1,2,4-oxadiazol-5-yl)methyl)-N-(pyrazin-2-yl)acrylamide;
2-((3-(4-butylbenzyl)-1,2,4-oxadiazol-5-yl)methyl)-N-(5-methylthiazol-2-yl)acrylamide;
2-methyloctan-2-yl 3-(pyrazin-2-ylcarbamoyl)but-3-enoate;
1-(4-(trifluoromethyl)phenyl)cyclobutyl 3-(methylcarbamoyl)but-3-enoate;
1-(4-(trifluoromethyl)phenyl)cyclobutyl 3-carbamoylbut-3-enoate;
(S)-1-(4-(trifluoromethyl)phenyl)ethyl 3-carbamoylbut-3-enoate;
(S)-1-(4-(trifluoromethyl)phenyl)ethyl 3-(methylcarbamoyl)but-3-enoate;
(R)-1-(4-(trifluoromethyl)phenyl)ethyl 3-(methylcarbamoyl)but-3-enoate;
(R)-1-(4-(trifluoromethyl)phenyl)ethyl 3-carbamoylbut-3-enoate;
3-(4-(trifluoromethyl)phenyl)oxetan-3-yl 3-(methylcarbamoyl)but-3-enoate;
3-(4-(trifluoromethyl)phenyl)oxetan-3-yl 3-carbamoylbut-3-enoate;
3-(5-(trifluoromethyl)pyridin-2-yl)oxetan-3-yl 3-(methylcarbamoyl)but-3-enoate;
3-(5-(trifluoromethyl)pyridin-2-yl)oxetan-3-yl 3-carbamoylbut-3-enoate;
1-(5-(trifluoromethyl)pyridin-2-yl)cyclobutyl 3-(methylcarbamoyl)but-3-enoate;
1-(5-(trifluoromethyl)pyridin-2-yl)cyclobutyl 3-carbamoylbut-3-enoate;
1-(3,5-dichlorophenyl)cyclobutyl 3-(methylcarbamoyl)but-3-enoate;
1-(3,5-dichlorophenyl)cyclobutyl 3-carbamoylbut-3-enoate;
1-(3-fluoro-4-(trifluoromethyl)phenyl)cyclobutyl 3-carbamoylbut-3-enoate;
1-(3-fluoro-4-(trifluoromethyl)phenyl)cyclobutyl 3-(methylcarbamoyl)but-3-enoate;
1-(4-(trifluoromethyl)phenyl)cyclobutyl 3-((1-methylpiperidin-4-yl)carbamoyl)but-3-enoate1-(4-(trifluoromethyl)phenyl)cyclobutyl 3-((2-morpholinoethyl)carbamoyl)but-3-enoate;
1-(4-(trifluoromethyl)phenyl)cyclobutyl 3-((1,1-dioxidothietan-3-yl)carbamoyl)but-3-enoate;
1-(4-(trifluoromethyl)phenyl)cyclobutyl 3-((2-(methylsulfonyl)ethyl)carbamoyl)but-3-enoate;
(2-((3-(1-(4-((trifluoromethyl)thio)phenyl)cyclopropyl)-1,2,4-oxadiazol-5-yl)methyl)acryloyl)glycine;
3,3,3-trifluoro-2-(2-((3-(1-(4-((trifluoromethyl)thio)phenyl)cyclopropyl)-1,2,4-oxadiazol-5-yl)methyl)acrylamido)propanoic acid;
(2-methylene-4-oxo-4-(1-(4-(trifluoromethyl)phenyl)cyclobutoxy)butanoyl) glycine;
   or a pharmaceutically acceptable salt and/or solvate of any one thereof.

Compounds of formula (I) may be prepared as set out in the Examples and as set out in the following schemes.

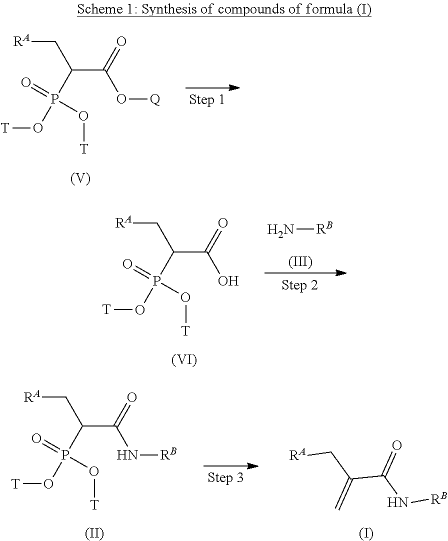

R^{A} and R^{B} are as defined elsewhere herein. Q is C₁₋₄ alkyl optionally substituted with halo, such as methyl, t-butyl or 2,2,2-trichloroethyl. T is C₁₋₄ alkyl such as ethyl.

Step 1: Compounds of formula (V) (whose synthesis is described below) may be converted via acid or base hydrolysis to compounds of formula (IV). For example, when Q is methyl, aq LiOH in THF may be used. When Q is t-butyl, trifluoroacetic acid (TFA) in dichloromethane (DCM) may be used. Appropriate conditions are well known to the skilled person.

In some cases, the alkyl or haloalkyl group Q may be replaced with an alternative protecting group such as 9H-fluoren-9-yl-methyl. In this case, removal of the protecting group may be achieved by treatment with a base such as triethylamine (TEA) in a solvent such as N,N-dimethylformamide. Appropriate conditions are well known to the skilled person.

Step 2: Compounds of formula (IV) may be converted to compounds of formula (II) by condensation with amine (Ill) and standard amide coupling conditions such as those described in the experimental section below. Other appropriate coupling conditions are well known to the skilled person.

Step 3: Compounds of formula (II) may be converted to compounds of formula (I) using a condensation reaction with formaldehyde or a formaldehyde equivalent thereof, e.g., paraformaldehyde.

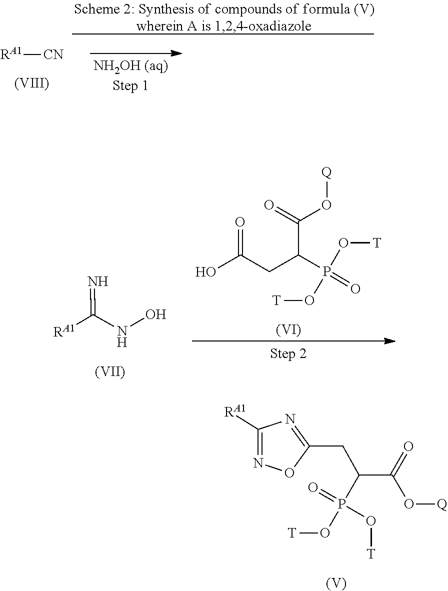

R^{A1}, T and Q are as defined elsewhere herein.

Step 1: Compounds of formula (VIII) may be converted to compounds of formula (VII) using aq. hydroxylamine in a solvent such as ethanol. Nitriles of formula (VIII) are commercially available or can be made in one step from commercially available starting materials, such as those described in the experimental section below.

Step 2: Amidoximes of formula (VII) may be converted to compounds of formula (V) by condensation with a compound of formula (VI), whose synthesis is shown below, in the presence of a coupling agent such as T3P, a base such as Et₃N, and a solvent such as ethyl acetate.

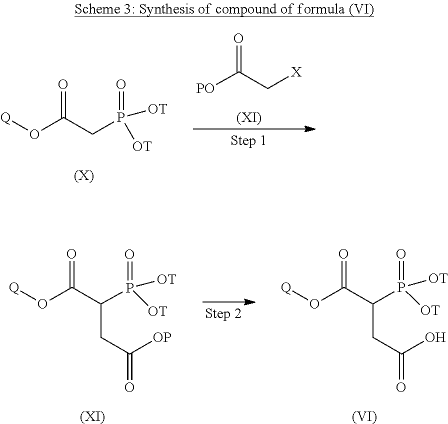

Q and T are as defined above. P is a carboxylic acid protecting group such as C₁₋₄ alkyl, e.g., ethyl. X is a leaving group, such as a halo atom, e.g., bromo.

Step 1: Compounds of formula (X) are alkylated with protected carboxy derivative (XI) under basic conditions (such as NaH in THF) to furnish compounds of formula (IX)).

Step 2: Protecting group P is removed under alkaline hydrolytic conditions, such as those described in the experimental section below, or conditions which are well known to the person skilled in the art.

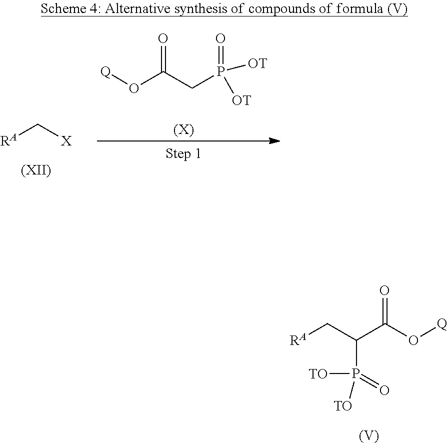

This scheme may be used for example when R^{A} is

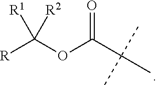

Q, T and X are as defined elsewhere herein.

Step 1: Compounds of formula (XII) can be converted to compounds of formula (V) by reaction with compounds of formula (X) under basic conditions (such as NaH in THF). Compounds of formula (XII) are commercially available or can be made in two steps from commercially available starting materials, using methods such as those described in the experimental section.

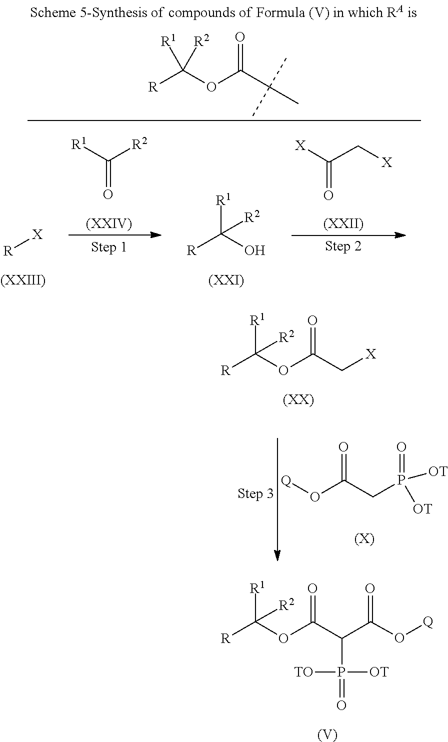

R, R¹ and R² are as defined for formula (I) and Q, T and X are as defined elsewhere herein.

Step 1: compounds of formula (XXIII) may be treated with a base such as n-butyl lithium (n-BuLi) in a solvent such as tetrahydrofuran (THF) at −78° C., followed by reaction with an aldehyde or ketone of formula (XXIV) to produce an alcohol of formula (XXI).

Alternatively, the leaving group X in the compound of formula (XXIII) may be replaced by MgX such that the compound is a Grignard reagent. In this case, the reaction may be conducted at about −5° to 5° C., typically 0° C. in a solvent such as THF.

Step 2: Esters of formula (XX) may be prepared by reacting a compound of formula (XXI) with a compound of formula (XXII), for example bromoacetyl bromide, in the presence of a base such as 1,8-Diazabicyclo[5.4.0]undec-7-ene (DBU).

Step 3: Compounds of formula (V) may be prepared by treating a compound of formula (XX) with a base such as sodium hydride followed by reaction with a compound of formula (X) as defined above.

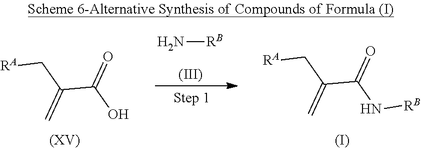

R^{A} and R^{B} are as defined for formula (I)

Step 1: there are a number of ways to prepare the compound of formula (I) from the compounds of formulae (XV) and (Ill).

A. The conversion may be achieved in two steps. In the first step, the carboxylic acid of formula (XV) is reacted with a halogenating agent to produce an acyl halide. Suitable halogenating agents include oxalyl chloride and 1-Chloro-N,N,2-trimethyl-1-propenylamine (Ghosez reagent). Reactions with oxalyl chloride or Ghosez reagent may take place at reduced temperature, for example about −5° to 5° C., typically 0° C. and in an organic solvent such as dichloromethane or tetrahydrofuran.

Secondly, the acyl halide is reacted with the compound of formula (III), suitably in the form of a salt such as the hydrochloride salt, and in the presence of a base such as triethylamine or aqueous potassium carbonate.

B. Alternatively, the compound of formula (XV) may be reacted with the compound of formula (III) in the presence of a coupling agent and a base. Suitable coupling agents are well known and include O-(Benzotriazol-1-yl)-N,N,N′,N′-tetramethyluronium hexafluorophosphate (HBTU), 0-(Benzotriazol-1-yl)-N,N,N′,N′-tetramethyluronium tetrafluoroborate (TBTU), 0-(7-Azabenzotriazol-1-yl)-N,N,N′,N′-tetramethyluronium hexafluorophosphate (HATU), 0-(7-Azabenzotriazol-1-yl)-N,N,N′,N′-tetramethyluronium tetrafluoroborate (TATU), (Benzotriazol-1-yloxy)tris(dimethylamino)phosphonium hexafluorophosphate (BOP), (Benzotriazol-1-yloxy)tripyrrolidinophosphonium hexafluorophosphate (PyBOP) carbodiimides such as 1-ethyl-3-(3-dimethylaminopropyl)carbodiimide (EDC) and triazoles such as 1-hydroxy-7-azabenzotriazole (HOAt) or hydroxybenzotriazole (HOBt).

Suitably, when these coupling agents are used, the reaction is conducted under basic conditions, for example in the presence of an amine such as triethylamine (TEA), diisopropylethylamine (DIPEA) or 4-dimethylaminopyridine (DMAP) and in an organic solvent such as N,N-dimethylformamide.

Alternatively, the coupling reagent may be propylphosphonic anhydride (T3P®). When T3P is used as the coupling reagent, the reaction may be conducted under basic conditions, for example in the presence of an amine such as diisopropylethylamine (DIPEA) or triethylamine (TEA) and in an organic solvent such as ethyl acetate.

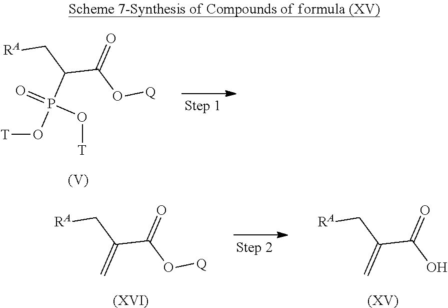

R^{A} and R^{B} are as defined for formula (I) and Q and T are as defined elsewhere herein.

Step 1: the compound of formula (V) is converted to a compound of formula (XVI) using a condensation reaction with formaldehyde or a formaldehyde equivalent thereof, e.g., paraformaldehyde.

Step 2: compounds of formula (XVI) is converted to compounds of formula (XV) by acid or base hydrolysis. For example, when Q is methyl, aq LiOH in THF may be used. When Q is t-butyl, TFA in DCM may be used. When Q is 2,2,2-trichloroethyl, zinc in acetic acid may be used. Appropriate conditions are well known to the skilled person.

In some cases, the alkyl or haloalkyl group Q may be replaced with an alternative protecting group such as 9H-fluoren-9-yl-methyl. In this case, removal of the protecting group may be achieved by treatment with a base such as triethylamine (TEA) in a solvent such as N,N-dimethylformamide. Appropriate conditions are well known to the skilled person.

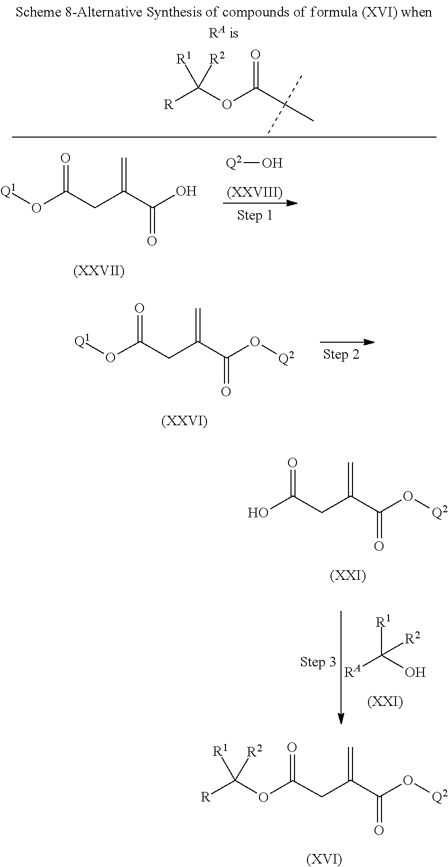

R, R¹ and R² are as defined for formula (I) and Q¹ and Q² are different groups Q, where Q is as defined elsewhere herein.

Step 1: a compound of formula (XXVI) may be prepared by reacting a compound of formula (XXVII) with a compound of formula (XXVIII) in the presence of a coupling reagent such as EDC in the presence of a base such as DMAP/DIPEA. Appropriate conditions are well known to the skilled person.

Step 2: since the groups Q¹ and Q² are orthogonal protecting groups, the compound of formula (XXV) may be prepared by selectively removing the group Q¹. For example, when Q¹ is p-methoxybenzyl and Q² is 2,2,2-dichloroethyl, the group Q¹ can be removed by hydrolysis, for example using TFA in DCM. Appropriate conditions are well known to the skilled person.

Step 3: the compound of formula (XVI) in which R^{A} is

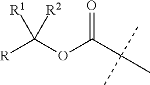

may be obtained by reaction of the carboxylic acid (XXV) with the alcohol (XXI) as defined above. Again, this reaction may be carried out in the presence of a coupling reagent such as EDC and a base such as DMAP/DIPEA. Appropriate conditions are well known to the skilled person.

In the above schemes, R^{C} and R^{D} are H. Such schemes may also be used to synthesise compounds of formula (I) wherein R^{C} and R^{D} are other than H. Compounds of formula (I) wherein

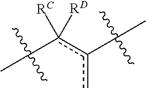

represents:

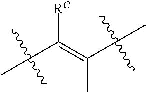

may be accessed via isomerisation of compounds of formula (I) wherein

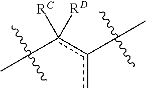

represents:

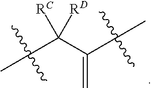

In an embodiment, there is provided a process for preparing a compound of formula (I) or a salt such as a pharmaceutically acceptable salt thereof, comprising the step of reacting a compound of formula (II):

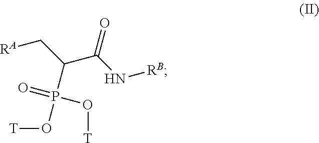

or a salt thereof;
with formaldehyde or a formaldehyde equivalent thereof, e.g., paraformaldehyde;
wherein R^{A}, R^{B} and T are defined elsewhere herein.

There is also provided a process for preparing a compound of formula (I) or a salt such as a pharmaceutically acceptable salt thereof, comprising the step of reacting a compound of formula (XV):

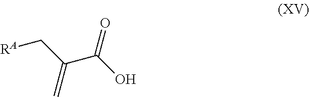

wherein R^{A} is as defined for formula (I);
with a halogenating agent to produce an acyl halide and reacting the acyl halide with a compound of formula (III):

H₂N—R^{B} (III)

wherein R^{B} is as defined in claim 1 or
reacting a compound of formula (XV) as defined above with a compound of formula (III) in the presence of a coupling agent and a base.

In one embodiment, there is provided a compound of formula (II):

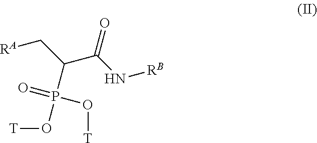

or a salt thereof, R^{A} and R^{B} are defined above, and T is C₁₋₄ alkyl such as ethyl.

There is also provided a compound of formula (XV):

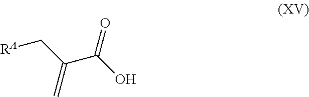

wherein R^{A} is as defined for formula (I).

It will be appreciated that for use in therapy the salts of the compounds of formula (I) should be pharmaceutically acceptable. Suitable pharmaceutically acceptable salts will be apparent to those skilled in the art. Pharmaceutically acceptable salts include acid addition salts, suitably salts of compounds of the invention comprising a basic group such as an amino group, formed with inorganic acids, e.g., hydrochloric acid, hydrobromic acid, sulfuric acid, nitric acid or phosphoric acid. Also included are salts formed with organic acids e.g. succinic acid, maleic acid, acetic acid, fumaric acid, citric acid, tartaric acid, benzoic acid, p-toluenesulfonic acid, methanesulfonic acid, naphthalenesulfonic acid and 1,5-naphthalenedisulfonic acid. Other salts, e.g., oxalates or formates, may be used, for example in the isolation of compounds of formula (I) and are included within the scope of this invention, as are basic addition salts such as sodium, potassium, calcium, aluminium, zinc, magnesium and other metal salts.

Pharmaceutically acceptable salts may also be formed with organic bases such as basic amines e.g. with ammonia, meglumine, tromethamine, piperazine, arginine, choline, diethylamine, benzathine or lysine. Thus, in one embodiment there is provided a compound of formula (I) in the form of a pharmaceutically acceptable salt. Alternatively, there is provided a compound of formula (I) in the form of a free acid. When the compound contains a basic group as well as the free acid it may be Zwitterionic.

Suitably, the compound of formula (I) is not a salt e.g. is not a pharmaceutically acceptable salt.

Suitably, where the compound of formula (I) is in the form of a salt, the pharmaceutically acceptable salt is a basic addition salt such as a carboxylate salt formed with a group 1 metal (e.g. a sodium or potassium salt), a group 2 metal (e.g. a magnesium or calcium salt) or an ammonium salt of a basic amine (e.g. an NH₄⁺ salt), such as a sodium salt.

The compounds of formula (I) may be prepared in crystalline or non-crystalline form and, if crystalline, may optionally be solvated, e.g. as the hydrate. This invention includes within its scope stoichiometric solvates (e.g. hydrates) as well as compounds containing variable amounts of solvent (e.g. water). Suitably, the compound of formula (I) is not a solvate.

The invention extends to a pharmaceutically acceptable derivative thereof, such as a pharmaceutically acceptable prodrug of compounds of formula (I). Typical prodrugs of compounds of formula (I) which comprise a carboxylic acid include ester (e.g. C₁₋₆ alkyl e.g. C₁₋₄ alkyl ester) derivatives thereof. Thus, in one embodiment, the compound of formula (I) is provided as a pharmaceutically acceptable prodrug. In another embodiment, the compound of formula (I) is not provided as a pharmaceutically acceptable prodrug.

It is to be understood that the present invention encompasses all isomers of compounds of formula (I) including all geometric, tautomeric and optical forms, and mixtures thereof (e.g. racemic mixtures). Where additional chiral centres are present in compounds of formula (I), the present invention includes within its scope all possible diastereoisomers, including mixtures thereof. The different isomeric forms may be separated or resolved one from the other by conventional methods, or any given isomer may be obtained by conventional synthetic methods or by stereospecific or asymmetric syntheses.

The present invention also includes all isotopic forms of the compounds provided herein, whether in a form (i) wherein all atoms of a given atomic number have a mass number (or mixture of mass numbers) which predominates in nature (referred to herein as the “natural isotopic form”) or (ii) wherein one or more atoms are replaced by atoms having the same atomic number, but a mass number different from the mass number of atoms which predominates in nature (referred to herein as an “unnatural variant isotopic form”). It is understood that an atom may naturally exist as a mixture of mass numbers. The term “unnatural variant isotopic form” also includes embodiments in which the proportion of an atom of given atomic number having a mass number found less commonly in nature (referred to herein as an “uncommon isotope”) has been increased relative to that which is naturally occurring e.g. to the level of >20%, >50%, >75%, >90%, >95% or >99% by number of the atoms of that atomic number (the latter embodiment referred to as an “isotopically enriched variant form”). The term “unnatural variant isotopic form” also includes embodiments in which the proportion of an uncommon isotope has been reduced relative to that which is naturally occurring. Isotopic forms may include radioactive forms (i.e. they incorporate radioisotopes) and non-radioactive forms. Radioactive forms will typically be isotopically enriched variant forms.

An unnatural variant isotopic form of a compound may thus contain one or more artificial or uncommon isotopes such as deuterium (²H or D), carbon-11 (¹¹C), carbon-13 (¹³C), carbon-14 (¹⁴C), nitrogen-13 (¹³N), nitrogen-15 (¹⁵N), oxygen-15 (¹⁵O), oxygen-17 (¹⁷O), oxygen-18 (¹⁸O), phosphorus-32 (³²P), sulphur-35 (³⁵S), chlorine-36 (³⁶Cl), chlorine-37 (³⁷Cl), fluorine-18 (¹⁸F) iodine-123 (¹²³I), iodine-125 (¹²⁵I) in one or more atoms or may contain an increased proportion of said isotopes as compared with the proportion that predominates in nature in one or more atoms.

Unnatural variant isotopic forms comprising radioisotopes may, for example, be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Unnatural variant isotopic forms which incorporate deuterium i.e. ²H or D may afford certain therapeutic advantages resulting from greater metabolic stability, for example, increased in vivo half-life or reduced dosage requirements, and hence may be preferred in some circumstances. Further, unnatural variant isotopic forms may be prepared which incorporate positron emitting isotopes, such as ¹¹C, ¹⁸F, ¹⁵O and ¹³N, and would be useful in positron emission topography (PET) studies for examining substrate receptor occupancy.

In one embodiment, the compounds of formula (I) are provided in a natural isotopic form. In one embodiment, the compounds of formula (I) are provided in an unnatural variant isotopic form. In a specific embodiment, the unnatural variant isotopic form is a form in which deuterium (i.e. ²H or D) is incorporated where hydrogen is specified in the chemical structure in one or more atoms of a compound of formula (I). In one embodiment, the atoms of the compounds of formula (I) are in an isotopic form which is not radioactive. In one embodiment, one or more atoms of the compounds of formula (I) are in an isotopic form which is radioactive. Suitably radioactive isotopes are stable isotopes. Suitably the unnatural variant isotopic form is a pharmaceutically acceptable form.

In one embodiment, a compound of formula (I) is provided whereby a single atom of the compound exists in an unnatural variant isotopic form. In another embodiment, a compound of formula (I) is provided whereby two or more atoms exist in an unnatural variant isotopic form.

Unnatural isotopic variant forms can generally be prepared by conventional techniques known to those skilled in the art or by processes described herein, e.g., processes analogous to those described in the accompanying Examples for preparing natural isotopic forms. Thus, unnatural isotopic variant forms could be prepared by using appropriate isotopically variant (or labelled) reagents in place of the normal reagents employed in the Examples. Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

### Therapeutic Indications

Compounds of formula (I) are of use in therapy, particularly for treating or preventing an inflammatory disease or a disease associated with an undesirable immune response. As shown in Biological Example 1 below, example compounds of formula (I) reduced cytokine release more effectively than dimethyl itaconate, 2-(2-chlorobenzyl)acrylic acid and 4-octyl itaconate as demonstrated by lower IC₅₀ values. Cytokines are important mediators of inflammation and immune-mediated disease as evidenced by the therapeutic benefit delivered by antibodies targeting them.

Thus, in a first aspect, the present invention provides a compound of formula (I) or a pharmaceutically acceptable salt and/or solvate thereof as defined herein, for use as a medicament. Also provided is a pharmaceutical composition comprising a compound of formula (I) or a pharmaceutically acceptable salt and/or solvate thereof as defined herein. Such a pharmaceutical composition contains the compound of formula (I) and a pharmaceutically acceptable carrier or excipient.

In a further aspect, the present invention provides a compound of formula (I) or a pharmaceutically acceptable salt and/or solvate thereof as defined herein, for use in treating or preventing an inflammatory disease or a disease associated with an undesirable immune response. In a further aspect, the present invention provides the use of a compound of formula (I) or a pharmaceutically acceptable salt and/or solvate thereof as defined herein, in the manufacture of a medicament for treating or preventing an inflammatory disease or a disease associated with an undesirable immune response. In a further aspect, the present invention provides a method of treating or preventing an inflammatory disease or a disease associated with an undesirable immune response, which comprises administering a compound of formula (I) or a pharmaceutically acceptable salt and/or solvate thereof as defined herein.

For all aspects of the invention, suitably the compound is administered to a subject in need thereof, wherein the subject is suitably a human subject.

In one embodiment is provided a compound of formula (I) or a pharmaceutically acceptable salt and/or solvate thereof as defined herein, for use in treating an inflammatory disease or disease associated with an undesirable immune response. In one embodiment of the invention is provided the use of a compound of formula (I) or a pharmaceutically acceptable salt and/or solvate thereof as defined herein, in the manufacture of a medicament for treating an inflammatory disease or a disease associated with an undesirable immune response. In one embodiment of the invention is provided a method of treating an inflammatory disease or a disease associated with an undesirable immune response, which comprises administering a compound of formula (I) or a pharmaceutically acceptable salt and/or solvate thereof as defined herein.

In one embodiment is provided a compound of formula (I) or a pharmaceutically acceptable salt and/or solvate thereof as defined herein, for use in preventing an inflammatory disease or a disease associated with an undesirable immune response. In one embodiment of the invention is provided the use of a compound of formula (I) or a pharmaceutically acceptable salt and/or solvate thereof as defined herein, in the manufacture of a medicament for preventing an inflammatory disease or a disease associated with an undesirable immune response. In one embodiment of the invention is provided a method of preventing an inflammatory disease or a disease associated with an undesirable immune response, which comprises administering a compound of formula (I) or a pharmaceutically acceptable salt and/or solvate thereof as defined herein.

In one embodiment is provided a compound of formula (I) or a pharmaceutically acceptable salt and/or solvate thereof as defined herein, for use in treating or preventing an inflammatory disease. In one embodiment of the invention is provided the use of a compound of formula (I) or a pharmaceutically acceptable salt and/or solvate thereof as defined herein, in the manufacture of a medicament for treating or preventing an inflammatory disease. In one embodiment of the invention is provided a method of treating or preventing an inflammatory disease, which comprises administering a compound of formula (I) or a pharmaceutically acceptable salt and/or solvate thereof as defined herein.

In one embodiment is provided a compound of formula (I) or a pharmaceutically acceptable salt and/or solvate thereof as defined herein, for use in treating or preventing a disease associated with an undesirable immune response. In one embodiment of the invention is provided the use of a compound of formula (I) or a pharmaceutically acceptable salt and/or solvate thereof as defined herein, in the manufacture of a medicament for treating or preventing a disease associated with an undesirable immune response. In one embodiment of the invention is provided a method of treating or preventing a disease associated with an undesirable immune response, which comprises administering a compound of formula (I) or a pharmaceutically acceptable salt and/or solvate thereof as defined herein.

An undesirable immune response will typically be an immune response which gives rise to a pathology i.e. is a pathological immune response or reaction.

In one embodiment, the inflammatory disease or disease associated with an undesirable immune response is an auto-immune disease.

In one embodiment, the inflammatory disease or disease associated with an undesirable immune response is, or is associated with, a disease selected from the group consisting of: psoriasis (including chronic plaque, erythrodermic, pustular, guttate, inverse and nail variants), asthma, chronic obstructive pulmonary disease (COPD, including chronic bronchitis and emphysema), heart failure (including left ventricular failure), myocardial infarction, angina pectoris, other atherosclerosis and/or atherothrombosis-related disorders (including peripheral vascular disease and ischaemic stroke), a mitochondrial and neurodegenerative disease (such as Parkinson's disease, Alzheimer's disease, Huntington's disease, amyotrophic lateral sclerosis, retinitis pigmentosa or mitochondrial encephalomyopathy), autoimmune paraneoplastic retinopathy, transplantation rejection (including antibody-mediated and T cell-mediated forms), multiple sclerosis, transverse myelitis, ischaemia-reperfusion injury (e.g. during elective surgery such as cardiopulmonary bypass for coronary artery bypass grafting or other cardiac surgery, following percutaneous coronary intervention, following treatment of acute ST-elevation myocardial infarction or ischaemic stroke, organ transplantation, or acute compartment syndrome), AGE-induced genome damage, an inflammatory bowel disease (e.g. Crohn's disease or ulcerative colitis), primary sclerosing cholangitis (PSC), PSC-autoimmune hepatitis overlap syndrome, non-alcoholic fatty liver disease (non-alcoholic steatohepatitis), rheumatica, granuloma annulare, cutaneous lupus erythematosus (CLE), systemic lupus erythematosus (SLE), lupus nephritis, drug-induced lupus, autoimmune myocarditis or myopericarditis, Dressler's syndrome, giant cell myocarditis, post-pericardiotomy syndrome, drug-induced hypersensitivity syndromes (including hypersensitivity myocarditis), eczema, sarcoidosis, erythema nodosum, acute disseminated encephalomyelitis (ADEM), neuromyelitis optica spectrum disorders, MOG (myelin oligodendrocyte glycoprotein) antibody-associated disorders (including MOG-EM), optic neuritis, CLIPPERS (chronic lymphocytic inflammation with pontine perivascular enhancement responsive to steroids), diffuse myelinoclastic sclerosis, Addison's disease, alopecia areata, ankylosing spondylitis, other spondyloarthritides (including peripheral spondyloarthritis, that is associated with psoriasis, inflammatory bowel disease, reactive arthritis or juvenile onset forms), antiphospholipid antibody syndrome, autoimmune hemolytic anaemia, autoimmune hepatitis, autoimmune inner ear disease, pemphigoid (including bullous pemphigoid, mucous membrane pemphigoid, cicatricial pemphigoid, herpes gestationis or pemphigoid gestationis, ocular cicatricial pemphigoid), linear IgA disease, Behçet's disease, celiac disease, Chagas disease, dermatomyositis, diabetes mellitus type I, endometriosis, Goodpasture's syndrome, Graves' disease, Guillain-Barre syndrome and its subtypes (including acute inflammatory demyelinating polyneuropathy, AIDP, acute motor axonal neuropathy (AMAN), acute motor and sensory axonal neuropathy (AMSAN), pharyngeal-cervical-brachial variant, Miller-Fisher variant and Bickerstaff's brainstem encephalitis), progressive inflammatory neuropathy, Hashimoto's disease, hidradenitis suppurativa, inclusion body myositis, necrotising myopathy, Kawasaki disease, IgA nephropathy, Henoch-Schonlein purpura, idiopathic thrombocytopenic purpura, thrombotic thrombocytopenic purpura (TTP), Evans' syndrome, interstitial cystitis, mixed connective tissue disease, undifferentiated connective tissue disease, morphea, myasthenia gravis (including MuSK antibody positive and seronegative variants), narcolepsy, neuromyotonia, pemphigus vulgaris, pernicious anaemia, psoriatic arthritis, polymyositis, primary biliary cholangitis (also known as primary biliary cirrhosis), rheumatoid arthritis, palindromic rheumatism, schizophrenia, autoimmune (meningo-)encephalitis syndromes, scleroderma, Sjogren's syndrome, stiff person syndrome, polymylagia rheumatica, giant cell arteritis (temporal arteritis), Takayasu arteritis, polyarteritis nodosa, Kawasaki disease, granulomatosis with polyangitis (GPA; formerly known as Wegener's granulomatosis), eosinophilic granulomatosis with polyangiitis (EGPA; formerly known as Churg-Strauss syndrome), microscopic polyarteritis/polyangiitis, hypocomplementaemic urticarial vasculitis, hypersensitivity vasculitis, cryoglobulinemia, thromboangiitis obliterans (Buerger's disease), vasculitis, leukocytoclastic vasculitis, vitiligo, acute disseminated encephalomyelitis, adrenoleukodystrophy, Alexander's disease, Alper's disease, balo concentric sclerosis or Marburg disease, cryptogenic organising pneumonia (formerly known as bronchiolitis obliterans organizing pneumonia), Canavan disease, central nervous system vasculitic syndrome, Charcot-Marie-Tooth disease, childhood ataxia with central nervous system hypomyelination, chronic inflammatory demyelinating polyneuropathy (CIDP), diabetic retinopathy, globoid cell leukodystrophy (Krabbe disease), graft-versus-host disease (GVHD) (including acute and chronic forms, as well as intestinal GVHD), hepatitis C (HCV) infection or complication, herpes simplex viral infection or complication, human immunodeficiency virus (HIV) infection or complication, lichen planus, monomelic amyotrophy, cystic fibrosis, pulmonary arterial hypertension (PAH, including idiopathic PAH), lung sarcoidosis, idiopathic pulmonary fibrosis, paediatric asthma, atopic dermatitis, allergic dermatitis, contact dermatitis, allergic rhinitis, rhinitis, sinusitis, conjunctivitis, allergic conjunctivitis, keratoconjunctivitis sicca, dry eye, xerophthalmia, glaucoma, macular oedema, diabetic macular oedema, central retinal vein occlusion (CRVO), macular degeneration (including dry and/or wet age related macular degeneration, AMD), post-operative cataract inflammation, uveitis (including posterior, anterior, intermediate and pan uveitis), iridocyclitis, scleritis, corneal graft and limbal cell transplant rejection, gluten sensitive enteropathy (coeliac disease), dermatitis herpetiformis, eosinophilic esophagitis, achalasia, autoimmune dysautonomia, autoimmune encephalomyelitis, autoimmune oophoritis, autoimmune orchitis, autoimmune pancreatitis, aortitis and periaortitis, autoimmune retinopathy, autoimmune urticaria, (idiopathic) Castleman's disease, Cogan's syndrome, IgG4-related disease, retroperitoneal fibrosis, juvenile idiopathic arthritis including systemic juvenile idiopathic arthritis (Still's disease), adult-onset Still's disease, ligneous conjunctivitis, Mooren's ulcer, pityriasis lichenoides et varioliformis acuta (PLEVA, also known as Mucha-Habermann disease), multifocal motor neuropathy (MMN), paediatric acute-onset neuropsychiatric syndrome (PANS) (including paediatric autoimmune neuropsychiatric disorders associated with streptococcal infections (PANDAS)), paraneoplastic syndromes (including paraneoplastic cerebellar degeneration, Lambert-Eaton myaesthenic syndrome, limbic encephalitis, brainstem encephalitis, opsoclonus myoclonus ataxia syndrome, anti-NMDA receptor encephalitis, thymoma-associated multiorgan autoimmunity), perivenous encephalomyelitis, reflex sympathetic dystrophy, relapsing polychondritis, sperm & testicular autoimmunity, Susac's syndrome, Tolosa-Hunt syndrome, Vogt-Koyanagi-Harada Disease, anti-synthetase syndrome, autoimmune enteropathy, immune dysregulation polyendocrinopathy enteropathy X-linked (IPEX), microscopic colitis, autoimmune lymphoproliferative syndrome (ALPS), autoimmune polyendocrinopathy-candidiasis-ectodermal dystrophy syndrome (APEX), gout, pseudogout, amyloid (including AA or secondary amyloidosis), eosinophilic fasciitis (Shulman syndrome) progesterone hypersensitivity (including progesterone dermatitis), familial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutieres syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria), Schnitzler syndrome; familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders (e.g. myocardial infarction, angina, ischaemic heart failure, ischaemic nephropathy, ischaemic stroke, peripheral vascular disease, aortic aneurysm), and renal inflammatory disorders (e.g. diabetic nephropathy, membranous nephropathy, minimal change disease, crescentic glomerulonephritis, acute kidney injury, renal transplantation).

In one embodiment, the inflammatory disease or disease associated with an undesirable immune response is, or is associated with, a disease selected from the following autoinflammatory diseases: familial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, and severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, and neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutieres syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria) and Schnitzler syndrome.

In one embodiment, the inflammatory disease or disease associated with an undesirable immune response is, or is associated with, a disease selected from the following diseases mediated by excess NF-κB or gain of function in the NF-κB signalling pathway or in which there is a major contribution to the abnormal pathogenesis therefrom (including non-canonical NF-κB signalling): familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders (e.g. myocardial infarction, angina, ischaemic heart failure, ischaemic nephropathy, ischaemic stroke, peripheral vascular disease, aortic aneurysm), renal inflammatory disorders (e.g. diabetic nephropathy, membranous nephropathy, minimal change disease, crescentic glomerulonephritis, acute kidney injury, renal transplantation), asthma, COPD, type 1 diabetes mellitus, rheumatoid arthritis, multiple sclerosis, inflammatory bowel disease (including ulcerative colitis and Crohn's disease), and SLE.

In one embodiment, the disease is selected from the group consisting of rheumatoid arthritis, psoriatic arthritis, ankylosing spondylitis, systemic lupus erythematosus, multiple sclerosis, psoriasis, Crohn's disease, ulcerative colitis, uveitis, cryopyrin-associated periodic syndromes, Muckle-Wells syndrome, juvenile idiopathic arthritis, chronic obstructive pulmonary disease and asthma.

In one embodiment, the disease is multiple sclerosis.

In one embodiment, the disease is psoriasis.

In one embodiment, the disease is asthma.

In one embodiment, the disease is chronic obstructive pulmonary disease.

In one embodiment, the disease is systemic lupus erythematosus.

### Administration

The compound of formula (I) is usually administered as a pharmaceutical composition. Thus, in one embodiment, is provided a pharmaceutical composition comprising a compound of formula (I) and one or more pharmaceutically acceptable diluents or carriers.

The compound of formula (I) may be administered by any convenient method, e.g. by oral, parenteral, buccal, sublingual, nasal, rectal, intrathecal or transdermal administration, and the pharmaceutical compositions adapted accordingly.

The compound of formula (I) may be administered topically to the target organ e.g. topically to the eye, lung, nose or skin. Hence the invention provides a pharmaceutical composition comprising a compound of formula (I) optionally in combination with one or more topically acceptable diluents or carriers.

A compound of formula (I) which is active when given orally can be formulated as a liquid or solid, e.g. as a syrup, suspension, emulsion, tablet, capsule or lozenge.

A liquid formulation will generally consist of a suspension or solution of the compound of formula (I) in a suitable liquid carrier(s). Suitably the carrier is non-aqueous e.g. polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatine capsule; alternatively, a dispersion or suspension can be prepared using any suitable pharmaceutical carrier(s), e.g. aqueous gums, celluloses, silicates or oils and the dispersion or suspension then filled into a soft gelatine capsule.

Typical parenteral compositions consist of a solution or suspension of the compound of formula (I) in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the compound of formula (I) in a pharmaceutically acceptable aqueous or non-aqueous solvent and are usually presented in single or multidose quantities in sterile form in a sealed container which can take the form of a cartridge or refill for use with an atomising device. Alternatively, the sealed container may be a disposable dispensing device such as a single dose nasal inhaler or an aerosol dispenser fitted with a metering valve. Where the dosage form comprises an aerosol dispenser, it will contain a propellant which can be a compressed gas e.g. air, or an organic propellant such as a chlorofluorocarbon (CFC) or a hydrofluorocarbon (HFC). Aerosol dosage forms can also take the form of pump-atomisers.

Topical administration to the lung may be achieved by use of an aerosol formulation. Aerosol formulations typically comprise the active ingredient suspended or dissolved in a suitable aerosol propellant, such as a chlorofluorocarbon (CFC) or a hydrofluorocarbon (HFC).

Topical administration to the lung may also be achieved by use of a non-pressurised formulation such as an aqueous solution or suspension. These may be administered by means of a nebuliser e.g. one that can be hand-held and portable or for home or hospital use (i.e. non-portable). The formulation may comprise excipients such as water, buffers, tonicity adjusting agents, pH adjusting agents, surfactants and co-solvents.

Topical administration to the lung may also be achieved by use of a dry-powder formulation. The formulation will typically contain a topically acceptable diluent such as lactose, glucose or mannitol (preferably lactose).

The compound of the invention may also be administered rectally, for example in the form of suppositories or enemas, which include aqueous or oily solutions as well as suspensions and emulsions and foams. Such compositions are prepared following standard procedures, well known by those skilled in the art. For example, suppositories can be prepared by mixing the active ingredient with a conventional suppository base such as cocoa butter or other glycerides. In this case, the drug is mixed with a suitable non-irritating excipient which is solid at ordinary temperatures but liquid at the rectal temperature and will therefore melt in the rectum to release the drug. Such materials are cocoa butter and polyethylene glycols.

Generally, for compositions intended to be administered topically to the eye in the form of eye drops or eye ointments, the total amount of the compound of the present invention will be about 0.0001 to less than 4.0% (w/w).

Preferably, for topical ocular administration, the compositions administered according to the present invention will be formulated as solutions, suspensions, emulsions and other dosage forms.

The compositions administered according to the present invention may also include various other ingredients, including, but not limited to, tonicity agents, buffers, surfactants, stabilizing polymer, preservatives, co-solvents and viscosity building agents. Suitable pharmaceutical compositions of the present invention include a compound of the invention formulated with a tonicity agent and a buffer. The pharmaceutical compositions of the present invention may further optionally include a surfactant and/or a palliative agent and/or a stabilizing polymer.

Various tonicity agents may be employed to adjust the tonicity of the composition, preferably to that of natural tears for ophthalmic compositions. For example, sodium chloride, potassium chloride, magnesium chloride, calcium chloride, simple sugars such as dextrose, fructose, galactose, and/or simply polyols such as the sugar alcohols mannitol, sorbitol, xylitol, lactitol, isomaltitol, maltitol, and hydrogenated starch hydrolysates may be added to the composition to approximate physiological tonicity. Such an amount of tonicity agent will vary, depending on the particular agent to be added. In general, however, the compositions will have a tonicity agent in an amount sufficient to cause the final composition to have an ophthalmically acceptable osmolality (generally about 150-450 mOsm, preferably 250-350 mOsm and most preferably at approximately 290 mOsm). In general, the tonicity agents of the invention will be present in the range of 2 to 4% w/w. Preferred tonicity agents of the invention include the simple sugars or the sugar alcohols, such as D-mannitol.

An appropriate buffer system (e.g. sodium phosphate, sodium acetate, sodium citrate, sodium borate or boric acid) may be added to the compositions to prevent pH drift under storage conditions. The particular concentration will vary, depending on the agent employed. Preferably however, the buffer will be chosen to maintain a target pH within the range of pH 5 to 8, and more preferably to a target pH of pH 5 to 7.

Surfactants may optionally be employed to deliver higher concentrations of compound of the present invention. The surfactants function to solubilise the compound and stabilise colloid dispersion, such as micellar solution, microemulsion, emulsion and suspension. Examples of surfactants which may optionally be used include polysorbate, poloxamer, polyosyl 40 stearate, polyoxyl castor oil, tyloxapol, Triton, and sorbitan monolaurate. Preferred surfactants to be employed in the invention have a hydrophile/lipophile/balance “HLB” in the range of 12.4 to 13.2 and are acceptable for ophthalmic use, such as TritonX114 and tyloxapol.

Additional agents that may be added to the ophthalmic compositions of compounds of the present invention are demulcents which function as a stabilising polymer. The stabilizing polymer should be an ionic/charged example with precedence for topical ocular use, more specifically, a polymer that carries negative charge on its surface that can exhibit a zeta-potential of (−)10-50 mV for physical stability and capable of making a dispersion in water (i.e. water soluble). A preferred stabilising polymer of the invention would be polyelectrolyte, or polyelectrolytes if more than one, from the family of cross-linked polyacrylates, such as carbomers and Pemulen®, specifically Carbomer 974p (polyacrylic acid), at 0.1-0.5% w/w.

Other compounds may also be added to the ophthalmic compositions of the compound of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

Topical ophthalmic products are typically packaged in multidose form. Preservatives are thus required to prevent microbial contamination during use. Suitable preservatives include: benzalkonium chloride, chlorobutanol, benzododecinium bromide, methyl paraben, propyl paraben, phenylethyl alcohol, edentate disodium, sorbic acid, polyquaternium-1, or other agents known to those skilled in the art. Such preservatives are typically employed at a level of from 0.001 to 1.0% w/v. Unit dose compositions of the present invention will be sterile, but typically unpreserved. Such compositions, therefore, generally will not contain preservatives.

Compositions suitable for buccal or sublingual administration include tablets, lozenges and pastilles where the compound of formula (I) is formulated with a carrier such as sugar and acacia, tragacanth, or gelatine and glycerine.

Compositions suitable for transdermal administration include ointments, gels and patches.

The composition may contain from 0.1% to 100% by weight, for example from 10% to 60% by weight, of the compound of formula (I), depending on the method of administration. The composition may contain from 0% to 99% by weight, for example 40% to 90% by weight, of the carrier, depending on the method of administration. The composition may contain from 0.05 mg to 1000 mg, for example from 1.0 mg to 500 mg, such as from 1.0 mg to 50 mg, e.g. about 10 mg of the compound of formula (I), depending on the method of administration. The composition may contain from 50 mg to 1000 mg, for example from 100 mg to 400 mg of the carrier, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 mg to 1000 mg, more suitably 1.0 mg to 500 mg, such as from 1.0 mg to 50 mg, e.g. about 10 mg and such unit doses may be administered more than once a day, for example two or three times a day. Such therapy may extend for a number of weeks or months.

In one embodiment of the invention, the compound of formula (I) is used in combination with a further therapeutic agent or agents. When the compound of formula (I) is used in combination with other therapeutic agents, the compounds may be administered either sequentially or simultaneously by any convenient route. Alternatively, the compounds may be administered separately.

Therapeutic agents which may be used in combination with the present invention include: corticosteroids (glucocorticoids), retinoids (e.g. acitretin, isotretinoin, tazarotene), anthralin, vitamin D analogues (e.g. cacitriol, calcipotriol), calcineurin inhibitors (e.g. tacrolimus, pimecrolimus), phototherapy or photochemotherapy (e.g. psoralen ultraviolet irradiation, PUVA) or other form of ultraviolet light irradiation therapy, ciclosporine, thiopurines (e.g. azathioprine, 6-mercaptopurine), methotrexate, anti-TNFα agents (e.g. infliximab, etanercept, adalimumab, certolizumab, golimumab and biosimilars), phosphodiesterase-4 (PDE4) inhibition (e.g. apremilast, crisaborole), anti-IL-17 agents (e.g. brodalumab, ixekizumab, secukinumab), anti-IL12/IL-23 agents (e.g. ustekinumab, briakinumab), anti-IL-23 agents (e.g. guselkumab, tildrakizumab), JAK (Janus Kinase) inhibitors (e.g. tofacitinib, ruxolitinib, baricitinib, filgotinib, upadacitinib), plasma exchange, intravenous immune globulin (IVIG), cyclophosphamide, anti-CD20 B cell depleting agents (e.g. rituximab, ocrelizumab, ofatumumab, obinutuzumab), anthracycline analogues (e.g. mitoxantrone), cladribine, sphingosine 1-phosphate receptor modulators or sphingosine analogues (e.g. fingolimod, siponimod, ozanimod, etrasimod), interferon beta preparations (including interferon beta 1b/1a), glatiramer, anti-CD3 therapy (e.g. OKT3), anti-CD52 targeting agents (e.g. alemtuzumab), leflunomide, teriflunomide, gold compounds, laquinimod, potassium channel blockers (e.g. dalfampridine/4-aminopyridine), mycophenolic acid, mycophenolate mofetil, purine analogues (e.g. pentostatin), mTOR (mechanistic target of rapamycin) pathway inhibitors (e.g. sirolimus, everolimus), anti-thymocyte globulin (ATG), IL-2 receptor (CD25) inhibitors (e.g. basiliximab, daclizumab), anti-IL-6 receptor or anti-IL-6 agents (e.g. tocilizumab, siltuximab), Bruton's tyrosine kinase (BTK) inhibitors (e.g. ibrutinib), tyrosine kinase inhibitors (e.g. imatinib), ursodeoxycholic acid, hydroxychloroquine, chloroquine, B cell activating factor (BAFF, also known as BLyS, B lymphocyte stimulator) inhibitors (e.g. belimumab, blisibimod), other B cell targeted therapy including fusion proteins targeting both APRIL (A PRoliferation-Inducing Ligand) and BLyS (e.g. atacicept), PI3K inhibitors including pan-inhibitors or those targeting the p110δ and/or p110γ containing isoforms (e.g. idelalisib, copanlisib, duvelisib), interferon α receptor inhibitors (e.g. anifrolumab, sifalimumab), T cell co-stimulation blockers (e.g. abatacept, belatacept), thalidomide and its derivatives (e.g. lenalidomide), dapsone, clofazimine, leukotriene antagonists (e.g. montelukast), theophylline, anti-IgE therapy (e.g. omalizumab), anti-IL-5 agents (e.g. mepolizumab, reslizumab), long-acting muscarinic agents (e.g. tiotropium, aclidinium, umeclidinium), PDE4 inhibitors (e.g. roflumilast), riluzole, free radical scavengers (e.g. edaravone), proteasome inhibitors (e.g. bortezomib), complement cascade inhibitors including those directed against C5 (e.g. eculizumab), immunoadsor, antithymocyte globulin, 5-aminosalicylates and their derivatives (e.g. sulfasalazine, balsalazide, mesalamine), anti-integrin agents including those targeting α4β1 and/or α4β7 integrins (e.g. natalizumab, vedolizumab), anti-CD11-α agents (e.g. efalizumab), non-steroidal anti-inflammatory drugs (NSAIDs) including the salicylates (e.g. aspirin), propionic acids (e.g. ibuprofen, naproxen), acetic acids (e.g. indomethacin, diclofenac, etodolac), oxicams (e.g. meloxicam) and fenamates (e.g. mefenamic acid), selective or relatively selective COX-2 inhibitors (e.g. celecoxib, etroxicoxib, valdecoxib and etodolac, meloxicam, nabumetone), colchicine, IL-4 receptor inhibitors (e.g. dupilumab), topical/contact immunotherapy (e.g. diphenylcyclopropenone, squaric acid dibutyl ester), anti-IL-1 receptor therapy (e.g. anakinra), IL-1P inhibitor (e.g. canakinumab), IL-1 neutralising therapy (e.g. rilonacept), chlorambucil, specific antibiotics with immunomodulatory properties and/or ability to modulate NRF2 (e.g. tetracyclines including minocycline, clindamycin, macrolide antibiotics), anti-androgenic therapy (e.g. cyproterone, spironolactone, finasteride), pentoxifylline, ursodeoxycholic acid, obeticholic acid, fibrate, cystic fibrosis transmembrane conductance regulator (CFTR) modulators, VEGF (vascular endothelial growth factor) inhibitors (e.g. bevacizumab, ranibizumab, pegaptanib, aflibercept), pirfenidone, and mizoribine.

Compounds of formula (I) may display one or more of the following desirable properties:
low IC₅₀ values for inhibiting release of cytokines, e.g., IL-1p and/or IL-6, from cells;
low EC₅₀ and/or high Eₘₐₓ values for activating the NRF2 pathway;
enhanced efficacy through improved hydrolytic stability and/or augmented maximum response;
reduced dose and dosing frequency through improved pharmacokinetics;
improved oral systemic bioavailability;
reduced plasma clearance following intravenous dosing;
augmented cell permeability;
enhanced aqueous solubility;
good tolerability, for example, by limiting the flushing and/or gastrointestinal side effects provoked by oral DMF (Hunt T. et al., 2015; WO2014/152494A1, incorporated herein by reference), possibly by reducing or eliminating HCA2 activity;
low toxicity at the relevant therapeutic dose;
distinct anti-inflammatory profiles resulting from varied electrophilicities, leading to differential targeting of the cysteine proteome (van der Reest J. et al., 2018) and, therefore, modified effects on gene activation).

The invention may be defined by the following clauses:

Clause 1. A compound of formula (I):

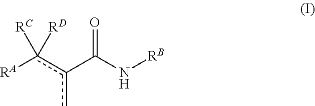

wherein,
R^{A} is:

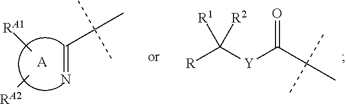

wherein when R^{A} is

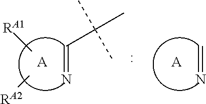

represents a 5 membered heteroaryl ring, which in addition to the C═N shown contains one or more further heteroatoms independently selected from N, O and S; or

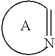

represents a 6 membered heteroaryl ring, which in addition to the C═N shown optionally contains one or more further N atoms;
R^{A1} is selected from the group consisting of C₁₋₁₀ alkyl, C₂₋₁₀ alkenyl, C₂₋₁₀ alkynyl, —(CH₂)₀₋₆—C₃₋₁₀-cycloalkyl, —(CH₂)₀₋₆—C₅₋₁₀ spirocycloalkyl, —(CH₂)₀₋₆-aryl and O-aryl;
   wherein R^{A1} is optionally substituted by one or more R^{A′} wherein each R^{A′} is independently selected from the group consisting of halo, C₁₋₆ alkyl, C₁₋₆ haloalkyl, hydroxy, cyano, OG¹, S(O)₀₋₂G¹, SF₅, (CH₂)₀₋₃C₃₋₇ cycloalkyl and 5-7-membered heterocyclyl wherein said C₃₋₇ cycloalkyl and said 5-7-membered heterocyclyl are optionally substituted by one or more R^{A″} wherein each R^{A″} is independently selected from the group consisting of halo, C₁₋₃ alkyl and C₁₋₃ haloalkyl; wherein two alkyl groups which are attached to the same carbon atom are optionally joined to form a C₃₋₇ cycloalkyl ring; wherein the C₃₋₁₀ cycloalkyl group is optionally fused to a phenyl ring which phenyl ring is optionally substituted by one or more halo atoms; and/or R^{A1} is optionally substituted by one phenyl ring which is optionally substituted by C₁₋₂ haloalkyl, C₁₋₂ haloalkoxy or one or more halo atoms;
   wherein, when two alkyl R^{A′} substituents are attached to a single carbon atom of R^{A′}, the two R^{A′} substituents may combine to form a 3- to 7-membered cycloalkyl ring;
   wherein G¹ is C₁₋₆ alkyl, C₃₋₇ cycloalkyl, C₁₋₆ haloalkyl, or (CH₂)₀₋₁phenyl wherein G¹ is optionally substituted by one or more G^{1′} wherein G^{1′} is independently selected from the group consisting of halo, C₁₋₂ alkyl, C₁₋₂ haloalkyl, hydroxy, cyano, nitro, C₁₋₂ alkoxy and C₁₋₂ haloalkoxy;
R^{A2} is selected from the group consisting of halo, C₁₋₆ alkyl, C₂₋₆ alkenyl, C₂₋₆ alkynyl, C₁₋₆ haloalkyl, hydroxy, cyano, nitro, NR³R⁴, OG² and S(O)₀₋₂G²;
   wherein G² is C₁₋₆ alkyl, C₃₋₇ cycloalkyl, C₁₋₆ haloalkyl, or phenyl which is optionally substituted by one or more G^{2′} wherein each G^{2′} is independently selected from the group consisting of halo, C₁₋₂ alkyl, C₁₋₂ haloalkyl, hydroxy, cyano, nitro, C₁₋₂ alkoxy and C₁₋₂ haloalkoxy; and
   wherein R³ and R⁴ are independently H or C₁₋₂ alkyl or, taken together, R³ and R⁴ may combine to form a 5-7-membered heterocyclic ring;
or R^{A2} is absent;
   wherein when R^{A} is

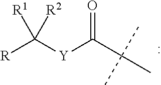

Y is O or NH; and
R is C₁₋₁₀ alkyl, and R¹ and R² are independently selected from the group consisting of H, C₁₋₄ alkyl and C₁₋₄ haloalkyl or R¹ and R² join to form a C₃₋₄ cycloalkyl ring; wherein R is optionally substituted by one or more R^{a} wherein each R^{a} is independently selected from the group consisting of halo, C₁₋₂ haloalkyl and C₁₋₂ haloalkoxy; or
R is selected from the group consisting of C₃₋₁₀ cycloalkyl, phenyl and 5- or 6-membered heteroaryl, and R¹ and R² are independently selected from the group consisting of H, C₁₋₄ alkyl and C₁₋₄ haloalkyl, or R¹ and R² join to form a C₃₋₄ cycloalkyl ring or a 4- to 6-membered heterocyclic ring, wherein the C₃₋₄ cycloalkyl ring or 4- to 6-membered heterocyclic ring is optionally substituted by methyl, halo or cyano; wherein R is optionally substituted by one or more R^{b} wherein each R^{b} is independently selected from the group consisting of halo, C₁₋₄ alkyl, C₁₋₄ haloalkyl, C₁₋₄ alkoxy, C₁₋₄ haloalkoxy and cyano; or
R is H, methyl or CF₃ and R¹ and R² are joined to form a C₃₋₁₀ cycloalkyl ring, wherein the C₃₋₁₀ cycloalkyl ring is optionally substituted by one or more R^{e} wherein each R^{e} is independently selected from the group consisting of halo, C₁₋₂ alkyl, C₁₋₂ haloalkyl, C₁₋₂ alkoxy and C₁₋₂ haloalkoxy, and/or wherein the C₃₋₁₀ cycloalkyl ring is optionally substituted by two R^{e} groups wherein the two R^{e} groups are attached to the same carbon atom and are joined to form a C₄₋₆ cycloalkyl ring;
R^{B} is heteroaryl wherein the heteroaryl is optionally substituted by one or more R^{B′} wherein each
R^{B′} is independently selected from the group consisting of halo, C₁₋₂ alkyl, C₁₋₂ haloalkyl, C₁₋₂ alkoxy, C₁₋₂ haloalkoxy, CO₂H, CO₂C₁₋₄ alkyl and NR⁵R⁶, wherein R⁵ and R⁶ are independently H, C₁₋₄ alkyl and C₃₋₆ cycloalkyl, or R⁵ and R⁶ join to form a 4-7-membered heterocyclic ring; or R^{B} is a 4- to 6-membered heterocyclic ring comprising one or two ring heteroatoms selected from N, O and S and optionally substituted on a ring nitrogen atom with C₁₋₂ alkyl and/or on a ring sulfur atom with one or two oxo substituents; or
R^{B} is selected from H, C₁₋₂ alkyl and C₁₋₂ haloalkyl, where alkyl and haloalkyl groups are optionally substituted with a substituent R^{B″};
wherein R^{B″} is selected from C(O)OH, C(O)O(C₁₋₂ alkyl), SO₂(C₁₋₂ alkyl), and a 5- or 6-membered heterocyclic ring comprising one or two ring heteroatoms selected from N, O and S and optionally substituted with C₁₋₂ alkyl; and
R^{C} and R^{D} are each independently H, C₁₋₂ alkyl, hydroxy, fluoro or C₁₋₂ alkoxy; or R^{C} and R^{D} may join to form a C₃₋₅ cycloalkyl ring;
wherein

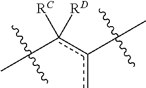

in the compound of formula (I) represents:

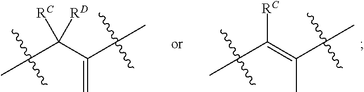

and wherein
   the total number of carbon atoms in groups R^{A′} and R^{A2} taken together including their optional substituents is 1 to 14; and
   wherein the total number of carbon atoms in groups R, R¹ and R² taken together, including their optional substituents, and including the carbon to which R, R¹ and R² are attached, is 3 to 14;
      or a pharmaceutically acceptable salt and/or solvate thereof.

The present invention provides a pharmaceutical composition comprising a compound of formula (I) or a pharmaceutically acceptable salt and/or solvate thereof.

Clause 2. The compound, pharmaceutically acceptable salt and/or solvate thereof according to clause 1, which is a compound of formula (IA):

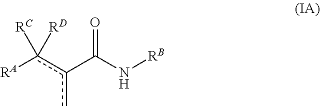

wherein,
R^{A} is

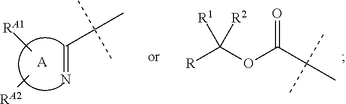

wherein when R^{A} is

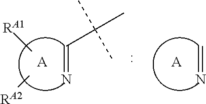

represents a 5 membered heteroaryl ring, which in addition to the C═N shown contains one or more further heteroatoms independently selected from N, O and S; or

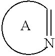

represents a 6 membered heteroaryl ring, which in addition to the C═N shown optionally contains one or more further N atoms:
R^{A1} is selected from the group consisting of C₁₋₁₀ alkyl, C₂₋₁₀ alkenyl, C₂₋₁₀ alkynyl, —(CH₂)₀₋₆—C₃₋₁₀-cycloalkyl, —(CH₂)₀₋₆—C₅₋₁₀ spirocycloalkyl, —(CH₂)₀₋₆-aryl and O-aryl;
   wherein R^{A1} is optionally substituted by one or more R^{A′} wherein R^{A′} is independently selected from the group consisting of halo, C₁₋₆ alkyl, C₁₋₆ haloalkyl, hydroxy, cyano, OG¹, S(O)₀₋₂G¹, SF₅, (CH₂)₀₋₃C₃₋₇ cycloalkyl and 5-7-membered heterocyclyl wherein said C₃₋₇ cycloalkyl and said 5-7-membered heterocyclyl are optionally substituted by one or more R^{A″} wherein R^{A″} is independently selected from the group consisting of halo, C₁₋₃ alkyl and C₁₋₃ haloalkyl; wherein two alkyl groups which are attached to the same carbon atom are optionally joined to form a C₃₋₇ cycloalkyl ring; wherein the C₃₋₁₀ cycloalkyl group is optionally fused to a phenyl ring which phenyl ring is optionally substituted by one or more halo atoms; and/or R^{A1} is optionally substituted by one phenyl ring which is optionally substituted by C₁₋₂ haloalkyl, C₁₋₂ haloalkoxy or one or more halo atoms;
   wherein G¹ is C₁₋₆ alkyl, C₃₋₇ cycloalkyl, C₁₋₆ haloalkyl, or (CH₂)₀₋₁phenyl wherein G¹ is optionally substituted by one or more G^{1′} wherein G^{1′} is independently selected from the group consisting of halo, C₁₋₂ alkyl, C₁₋₂ haloalkyl, hydroxy, cyano, nitro, C₁₋₂ alkoxy and C₁₋₂ haloalkoxy;
R^{A2} is selected from the group consisting of halo, C₁₋₆ alkyl, C₂₋₆ alkenyl, C₂₋₆ alkynyl, C₁-haloalkyl, hydroxy, cyano, nitro, NR³R⁴, OG² and S(O)₀₋₂G²;
   wherein G² is C₁₋₆ alkyl, C₃₋₇ cycloalkyl, C₁₋₆ haloalkyl, or phenyl which is optionally substituted by one or more G^{2′} wherein G^{2′} is independently selected from the group consisting of halo, C₁₋₂ alkyl, C₁₋₂ haloalkyl, hydroxy, cyano, nitro, C₁₋₂ alkoxy and C₁₋₂ haloalkoxy; and
   wherein R³ and R⁴ are independently H or C₁₋₂ alkyl or, taken together, R³ and R⁴ may combine to form a 5-7-membered heterocyclic ring;
or R^{A2} is absent;
   wherein when R^{A} is

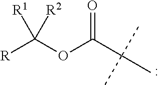

R is C₁₋₁₀ alkyl, and R¹ and R² are independently selected from the group consisting of H, C₁₋₄ alkyl and C₁₋₄ haloalkyl or R¹ and R² join to form a C₃₋₄ cycloalkyl ring; wherein R is optionally substituted by one or more R^{a} wherein R^{a} is independently selected from the group consisting of halo, C₁₋₂ haloalkyl and C₁₋₂ haloalkoxy; or
R is selected from the group consisting of C₃₋₁₀ cycloalkyl, phenyl and 5- or 6-membered heteroaryl, and R¹ and R² are independently selected from the group consisting of H, C₁₋₄ alkyl and C₁₋₄ haloalkyl, or R¹ and R² join to form a C₃₋₄ cycloalkyl ring or a C₄₋₆ heterocycloalkyl ring, wherein the C₃₋₄ cycloalkyl ring is optionally substituted by methyl, halo or cyano; wherein R is optionally substituted by one or more R^{b} wherein R^{b} is independently selected from the group consisting of halo, C₁₋₄ alkyl, C₁₋₄ haloalkyl, C₁₋₄ alkoxy, C₁₋₄ haloalkoxy and cyano; or
R is H, methyl or CF₃ and R¹ and R² are joined to form a C₃₋₁₀ cycloalkyl ring, wherein the C₃₋₁₀ cycloalkyl ring is optionally substituted by one or more R^{e} wherein R^{e} is independently selected from the group consisting of halo, C₁₋₂ alkyl, C₁₋₂ haloalkyl, C₁₋₂ alkoxy and C₁₋₂ haloalkoxy, and/or wherein the C₃₋₁₀ cycloalkyl ring is optionally substituted by two R^{e} groups wherein the two R^{e} groups are attached to the same carbon atom and are joined to form a C₄₋₆ cycloalkyl ring;
R^{B} is heteroaryl wherein the heteroaryl is optionally substituted by one or more R^{B′} wherein R^{B′} is independently selected from the group consisting of halo, C₁₋₂ alkyl, C₁₋₂ haloalkyl, C₁₋₂ alkoxy, C₁₋₂ haloalkoxy, CO₂H, CO₂C₁₋₄ alkyl and NR⁵R⁶, wherein R⁵ and R⁶ are independently H, C₁₋₄ alkyl and C₃₋₆ cycloalkyl, or R⁵ and R⁶ join to form a 4-7-membered heterocyclic ring; and
R^{C} and R^{D} are each independently H, C₁₋₂ alkyl, hydroxy, fluoro or C₁₋₂ alkoxy; or R^{C} and R^{D} may join to form a C₃₋₅ cycloalkyl ring,
wherein

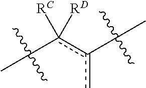

in the compound of formula (I) represents:

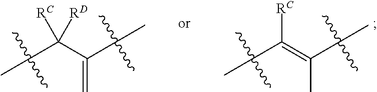

and wherein
   the total number of carbon atoms in groups R^{A} and R^{A2} taken together including their optional substituents is 1 to 14; and
   wherein the total number of carbon atoms in groups R, R¹ and R² taken together, including their optional substituents, and including the carbon to which R, R¹ and R² are attached, is 3 to 14;
or a pharmaceutically acceptable salt and/or solvate thereof.

Clause 3. The compound, pharmaceutically acceptable salt and/or solvate thereof according to clause 1 or clause 1 wherein R^{A} is

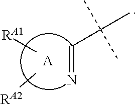

Clause 4. The compound, pharmaceutically acceptable salt and/or solvate thereof according to clause 3 wherein

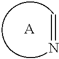

represents a 5 membered heteroaryl ring, which in addition to the C═N shown contains one or more (e.g., one or two) further heteroatoms independently selected from N, O and S.

Clause 5. The compound, pharmaceutically acceptable salt and/or solvate thereof according to clause 4 wherein the 5 membered ring is 1,2,4-oxadiazole.

Clause 6. The compound, pharmaceutically acceptable salt and/or solvate thereof according to clause 3 wherein

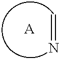

represents a 6 membered heteroaryl ring, which in addition to the C═N shown optionally contains one or more (e.g., one or two) further N atoms.

Clause 7. The compound, pharmaceutically acceptable salt and/or solvate thereof according to any one of clauses 1 to 6 wherein R^{A1} is —(CH₂)₀₋₆-aryl.

Clause 8. The compound, pharmaceutically acceptable salt and/or solvate thereof according to clause 7 wherein R^{A1} is CH₂aryl wherein aryl is phenyl.

Clause 9. The compound, pharmaceutically acceptable salt and/or solvate thereof according to any one of clauses 1 to 8 wherein R^{A1} is not substituted.

Clause 10. The compound, pharmaceutically acceptable salt and/or solvate thereof according to any one of clauses 1 to 8 wherein R^{A1} is substituted by one or more (such as one, two or three, e.g. one) R^{A′}.

Clause 11. The compound, pharmaceutically acceptable salt and/or solvate thereof according to clause 10 wherein R^{A′} is independently selected from the group consisting of C₁₋₆ alkyl and S(O)₀₋₂G¹.

Clause 12. The compound, pharmaceutically acceptable salt and/or solvate thereof according to clause 11 wherein R^{A′} is C₁₋₆ alkyl such as C₄ alkyl.

Clause 13. The compound, pharmaceutically acceptable salt and/or solvate thereof according to clause 11 wherein R^{A′} is S(O)₀₋₂G¹.

Clause 14. The compound, pharmaceutically acceptable salt and/or solvate thereof according to clause 13 wherein R^{A′} is SG¹ wherein G¹ is C₁₋₆ alkyl, C₃₋₇ cycloalkyl, C₁₋₆ haloalkyl, or (CH₂)₀₋₁ phenyl wherein G¹ is optionally substituted by one or more G^{1′}.

Clause 15. The compound, pharmaceutically acceptable salt and/or solvate thereof according to clause 13 or 14 wherein G¹ is not substituted.

Clause 16. The compound, pharmaceutically acceptable salt and/or solvate thereof according to clause 13 or 14 wherein G¹ is substituted by one or more (such as one, two or three, e.g. one) G^{1′}.

Clause 17. The compound, pharmaceutically acceptable salt and/or solvate thereof according to any one of clauses 1 to 16 wherein G¹ is C₁₋₆ haloalkyl such as CF₃.

Clause 18. The compound, pharmaceutically acceptable salt and/or solvate thereof according to any one of clauses 1 to 10 wherein said C₃₋₇ cycloalkyl and said 5-7-membered heterocyclyl are not substituted.

Clause 19. The compound, pharmaceutically acceptable salt and/or solvate thereof according to any one of clauses 1 to 10 wherein said C₃₋₇ cycloalkyl and said 5-7-membered heterocyclyl are substituted by one or more (such as one, two or three e.g. one) R^{A″}.

Clause 20. The compound, pharmaceutically acceptable salt and/or solvate thereof according to any one of clauses 1 to 18 wherein two alkyl groups R^{A′} which are attached to the same carbon atom are optionally joined (e.g. are joined) to form a C₃₋₇ cycloalkyl ring.

Clause 21. The compound, pharmaceutically acceptable salt and/or solvate thereof according to clause 20 wherein R^{A1} is —CH₂-aryl and wherein two methyl R^{A′} groups are attached to the CH₂ carbon of CH₂aryl and are joined to form a cyclopropyl ring.

Clause 22. The compound, pharmaceutically acceptable salt and/or solvate thereof according to any one of clauses 1 to 21 wherein R^{A1} is optionally substituted by one phenyl ring which is optionally substituted by C₁₋₂ haloalkyl, C₁₋₂ haloalkoxy or one or more (such as one, two or three e.g. one) halo atoms.

Clause 23. The compound, pharmaceutically acceptable salt and/or solvate thereof according to any one of clauses 1 to 22 wherein R^{A1} is substituted by two alkyl groups (such as two methyl groups) which are attached to the same carbon atom are optionally joined (e.g. are joined) to form a C₃₋₇ cycloalkyl ring (such as a cyclopropyl ring) and R^{A1} is additionally substituted by one or more R^{A′}, wherein R^{A′} is C₁₋₆ alkyl such as C₄ alkyl or S(O)₀₋₂G¹ such as SG¹ wherein G¹ is suitably C₁₋₆ haloalkyl such as CF₃.

Clause 24. The compound, pharmaceutically acceptable salt and/or solvate thereof according to any one of clauses 1 to 23 wherein R^{A2} is absent.

Clause 25. The compound, pharmaceutically acceptable salt and/or solvate thereof according to clause 1 wherein R^{A} is

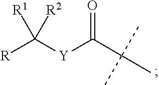

wherein Y, R, R¹ and R² are as defined in clause 1.

Clause 26. The compound, pharmaceutically acceptable salt and/or solvate thereof according to clause 25 wherein Y is O.

Clause 27. The compound, pharmaceutically acceptable salt and/or solvate thereof according to clause 25 wherein Y is NH.

Clause 28. The compound, pharmaceutically acceptable salt and/or solvate thereof according to any one of clauses 25 to 27 wherein R is C₁₋₁₀ alkyl, and R¹ and R² are independently selected from the group consisting of H, C₁₋₄ alkyl and C₁₋₄ haloalkyl or R¹ and R² join to form a C₃₋₄ cycloalkyl ring.

Clause 29. The compound, pharmaceutically acceptable salt and/or solvate thereof according to clause 28 wherein R is C₆₋₁₀ alkyl such as Cᵣ alkyl.

Clause 30. The compound, pharmaceutically acceptable salt and/or solvate thereof according to clause 28 or clause 29 wherein R¹ and R² are independently selected from the group consisting of H, C₁₋₄ alkyl and C₁₋₄ haloalkyl.

Clause 31. The compound, pharmaceutically acceptable salt and/or solvate thereof according to clause 30 wherein R₁ and R₂ are independently C₁₋₄ alkyl such as methyl.

Clause 32. The compound, pharmaceutically acceptable salt and/or solvate thereof according to clause 28 or clause 29 wherein R¹ and R² join to form a C₃₋₄ cycloalkyl ring.

Clause 33. The compound, pharmaceutically acceptable salt and/or solvate thereof according to any one of clauses 25 to 32 wherein R is not substituted.

Clause 34. The compound, pharmaceutically acceptable salt and/or solvate thereof according to any one of clauses 25 to 32 wherein R is substituted by one or more (such as one, two, or three, e.g. one) R^{a}.

Clause 35. The compound, pharmaceutically acceptable salt and/or solvate thereof according to clause 25 wherein R is selected from the group consisting of C₃₋₁₀ cycloalkyl, phenyl and 5- or 6-membered heteroaryl, and R¹ and R² are independently selected from the group consisting of H, C₁₋₄ alkyl and C₁₋₄ haloalkyl, or R¹ and R² join to form a C₃₋₄ cycloalkyl ring or a C₄₋₆ heterocycloalkyl ring, wherein the C₃₋₄ cycloalkyl ring is optionally substituted by methyl, halo or cyano.

Clause 36. The compound, pharmaceutically acceptable salt and/or solvate thereof according to clause 35 wherein R is C₃₋₁₀ cycloalkyl.

Clause 37. The compound, pharmaceutically acceptable salt and/or solvate thereof according to clause 35 wherein R is phenyl.

Clause 38. The compound, pharmaceutically acceptable salt and/or solvate thereof according to clause 35 wherein R is 5- or 6-membered heteroaryl, for example pyridyl.

Clause 39. The compound, pharmaceutically acceptable salt and/or solvate thereof according to any one of clauses 35 to 38 wherein R¹ and R² are independently selected from the group consisting of H, C₁₋₄ alkyl and C₁₋₄ haloalkyl.

Clause 40. The compound, pharmaceutically acceptable salt and/or solvate thereof according to any one of clauses 35 to 38 wherein R¹ and R² join to form a C₃₋₄ cycloalkyl ring.

Clause 41. The compound, pharmaceutically acceptable salt and/or solvate thereof according to clause 40 wherein the C₃₋₄ cycloalkyl ring is not substituted.

Clause 42. The compound, pharmaceutically acceptable salt and/or solvate thereof according to clause 40 wherein the C₃₋₄ cycloalkyl ring is substituted by methyl, halo or cyano.

Clause 43. The compound, pharmaceutically acceptable salt and/or solvate thereof according to any one of clauses 35 to 38 wherein R¹ and R² join to form a 4- to 6-membered heterocyclic ring.

Clause 44. The compound, pharmaceutically acceptable salt and/or solvate thereof according to any one of clauses 35 to 43 wherein R is not substituted.

Clause 45. The compound, pharmaceutically acceptable salt and/or solvate thereof according to any one of clauses 35 to 43 wherein R is substituted by one or more (such as one, two or three e.g. one) R^{b}.

Clause 46. The compound, pharmaceutically acceptable salt and/or solvate thereof according to clause 45 wherein R is phenyl or pyridyl and is substituted by one R^{b}, which is at the para position of the phenyl or pyridyl ring with respect to the linkage to —C(R¹)(R²)—.

Clause 47. The compound, pharmaceutically acceptable salt and/or solvate thereof according to clause 46 wherein R^{b} is trifluoromethyl.

Clause 48. The compound, pharmaceutically acceptable salt and/or solvate thereof according to any one of clauses 25 to 27 wherein wherein R is H, methyl or CF₃ and R¹ and R² are joined to form a C₃₋₁₀ cycloalkyl ring.

Clause 49. The compound, pharmaceutically acceptable salt and/or solvate thereof according to clause 48 wherein R is H.

Clause 50. The compound, pharmaceutically acceptable salt and/or solvate thereof according to clause 48 wherein R is methyl.

Clause 51. The compound, pharmaceutically acceptable salt and/or solvate thereof according to clause 48 wherein R is cyano.

Clause 52. The compound, pharmaceutically acceptable salt and/or solvate thereof according to any one of clauses 48 to 51 wherein the C₃₋₁₀ cycloalkyl ring is not substituted.

Clause 53. The compound, pharmaceutically acceptable salt and/or solvate thereof according to any one of clauses 48 to 51 wherein the C₃₋₁₀ cycloalkyl ring is substituted by one or more (such as one, two or three e.g. one) R^{e} wherein R^{e} is independently selected from the group consisting of halo, C₁₋₂ alkyl, C₁₋₂ haloalkyl, C₁₋₂ alkoxy and C₁₋₂ haloalkoxy.

Clause 54. The compound, pharmaceutically acceptable salt and/or solvate thereof according to any one of clauses 48 to 51 and 53 wherein the C₃₋₁₀ cycloalkyl ring is substituted by two R^{e} groups wherein the two R^{e} groups are attached to the same carbon atom and are joined to form a C₄₋₆ cycloalkyl ring.

Clause 55. The compound, pharmaceutically acceptable salt and/or solvate thereof according to any one of clauses 1 to 54 wherein R^{B} is heteroaryl wherein the heteroaryl is optionally substituted by one or more R^{B′}.

Clause 56. The compound, pharmaceutically acceptable salt and/or solvate thereof according to clause 55 wherein R^{B} is thiazolyl.

Clause 57. The compound, pharmaceutically acceptable salt and/or solvate thereof according to clause 55 wherein R^{B} is pyridinyl.

Clause 58. The compound, pharmaceutically acceptable salt and/or solvate thereof according to clause 55 wherein R^{B} is pyrazinyl.

Clause 59. The compound, pharmaceutically acceptable salt and/or solvate thereof according to clause 55 wherein R^{B} is pyrimidinyl.

Clause 60. The compound, pharmaceutically acceptable salt and/or solvate thereof according to any one of clauses 55 to 59 wherein R^{B} is not substituted.

Clause 61. The compound, pharmaceutically acceptable salt and/or solvate thereof according to any one of clauses 55 to 59 wherein R^{B} is substituted by one or more (such as one, two or three e.g. one) R^{B}.

Clause 62. The compound, pharmaceutically acceptable salt and/or solvate thereof according to clause 61 wherein R^{B′} is C₁₋₂ alkyl e.g. methyl.

Clause 63. The compound, pharmaceutically acceptable salt and/or solvate thereof according to any one of clauses 1 to 54 wherein R^{B} is a 4- to 6-membered heterocyclic ring comprising one or two ring heteroatoms selected from N, O and S and optionally substituted on a ring nitrogen atom with C₁₋₂ alkyl and/or on a ring sulfur atom with one or two oxo substituents.

Clause 64. The compound, pharmaceutically acceptable salt and/or solvate thereof according to clause 63 wherein R^{B} is selected from piperidinyl, N-methyl-piperidinyl, pyrrolidinyl, azetidinyl, morpholinyl, thietane-1,1-dioxide and thietane-1-oxide.

Clause 65. The compound, pharmaceutically acceptable salt and/or solvate thereof according to any one of clauses 1 to 54 wherein R^{B} is selected from H, C₁₋₂ alkyl and C₁₋₂ haloalkyl, where alkyl and haloalkyl groups are optionally substituted with a substituent R^{B″}, wherein R^{B″} is as defined in clause 1.

Clause 66. The compound, pharmaceutically acceptable salt and/or solvate thereof according to clause 65, wherein R^{B} is H.

Clause 67. The compound, pharmaceutically acceptable salt and/or solvate thereof according to clause 65, wherein R^{B} is selected from C₁₋₂ alkyl and C₁₋₂ haloalkyl optionally substituted with a substituent R^{B″} as defined in clause 1.

Clause 68. The compound, pharmaceutically acceptable salt and/or solvate thereof according to clause 67, wherein R^{B} is unsubstituted C₁₋₂ alkyl or C₁₋₂ haloalkyl.

Clause 69. The compound, pharmaceutically acceptable salt and/or solvate thereof according to clause 67, wherein R^{B} is C₁₋₂ alkyl or C₁₋₂ haloalkyl substituted with a substituent R^{B″} as defined in clause 1.

Clause 70. The compound, pharmaceutically acceptable salt and/or solvate thereof according to clause 69, wherein R^{B″} is selected from C(O)OH, SO₂(methyl), and a 5- to 6-membered heterocyclic ring comprising a ring nitrogen atom and optionally one additional ring heteroatom.

Clause 71. The compound, pharmaceutically acceptable salt and/or solvate thereof according to any one of clauses 1 to 70 wherein R^{C} and R^{D} are each independently H, C₁₋₂ alkyl, hydroxy, fluoro or C₁₋₂ alkoxy.

Clause 72. The compound, pharmaceutically acceptable salt and/or solvate thereof according to clause 71 wherein R^{C} and R^{D} are H.

Clause 73. The compound, pharmaceutically acceptable salt and/or solvate thereof according to any one of clauses 1 to 70 wherein R^{C} and R^{D} may join to form a C₃₋₅ cycloalkyl ring.

Clause 74. The compound, pharmaceutically acceptable salt and/or solvate thereof according to any one of clauses 1 to 73 wherein

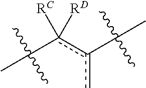

represents

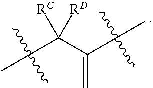

Clause 75. The compound, pharmaceutically acceptable salt and/or solvate thereof according to any one of clauses 1 to 73 wherein

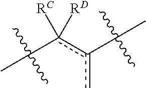

represents

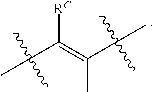

Clause 76. The compound, pharmaceutically acceptable salt and/or solvate thereof according to any one of clauses 1 to 75 wherein the compound of formula (I) is in the form of a salt, such as a pharmaceutically acceptable salt.

Clause 77. The compound, pharmaceutically acceptable salt and/or solvate thereof according to any one of clauses 1 to 75 wherein the compound of formula (I) is not in the form of a salt, such as a pharmaceutically acceptable salt.

Clause 78. The compound according to clause 1, which is selected from the list consisting of:
N-(5-methylthiazol-2-yl)-2-((3-(1-(4-((trifluoromethyl)thio)phenyl)cyclopropyl)-1,2,4-oxadiazol-5-yl)methyl)acrylamide;
N-(pyrazin-2-yl)-2-((3-(1-(4-((trifluoromethyl)thio)phenyl)cyclopropyl)-1,2,4-oxadiazol-5-yl)methyl)acrylamide;
2-((3-(4-butylbenzyl)-1,2,4-oxadiazol-5-yl)methyl)-N-(pyridin-2-yl)acrylamide;
2-((3-(4-butylbenzyl)-1,2,4-oxadiazol-5-yl)methyl)-N-(pyrazin-2-yl)acrylamide;
2-((3-(4-butylbenzyl)-1,2,4-oxadiazol-5-yl)methyl)-N-(5-methylthiazol-2-yl)acrylamide;
2-methyloctan-2-yl 3-(pyrazin-2-ylcarbamoyl)but-3-enoate;
1-(4-(trifluoromethyl)phenyl)cyclobutyl 3-(methylcarbamoyl)but-3-enoate;
1-(4-(trifluoromethyl)phenyl)cyclobutyl 3-carbamoylbut-3-enoate;
(S)-1-(4-(trifluoromethyl)phenyl)ethyl 3-carbamoylbut-3-enoate;
(S)-1-(4-(trifluoromethyl)phenyl)ethyl 3-(methylcarbamoyl)but-3-enoate;
(R)-1-(4-(trifluoromethyl)phenyl)ethyl 3-(methylcarbamoyl)but-3-enoate;
(R)-1-(4-(trifluoromethyl)phenyl)ethyl 3-carbamoylbut-3-enoate;
3-(4-(trifluoromethyl)phenyl)oxetan-3-yl 3-(methylcarbamoyl)but-3-enoate;
3-(4-(trifluoromethyl)phenyl)oxetan-3-yl 3-carbamoylbut-3-enoate;
3-(5-(trifluoromethyl) pyridin-2-yl)oxetan-3-yl 3-(methylcarbamoyl)but-3-enoate;
3-(5-(trifluoromethyl)pyridin-2-yl)oxetan-3-yl 3-carbamoylbut-3-enoate;
1-(5-(trifluoromethyl)pyridin-2-yl)cyclobutyl 3-(methylcarbamoyl)but-3-enoate;
1-(5-(trifluoromethyl)pyridin-2-yl)cyclobutyl 3-carbamoylbut-3-enoate;
1-(3,5-dichlorophenyl)cyclobutyl 3-(methylcarbamoyl)but-3-enoate;
1-(3,5-dichlorophenyl)cyclobutyl 3-carbamoylbut-3-enoate;
1-(3-fluoro-4-(trifluoromethyl)phenyl)cyclobutyl 3-carbamoylbut-3-enoate;
1-(3-fluoro-4-(trifluoromethyl)phenyl)cyclobutyl 3-(methylcarbamoyl)but-3-enoate;
1-(4-(trifluoromethyl)phenyl)cyclobutyl 3-((1-methylpiperidin-4-yl)carbamoyl)but-3-enoate;
1-(4-(trifluoromethyl)phenyl)cyclobutyl 3-((2-morpholinoethyl)carbamoyl)but-3-enoate;
1-(4-(trifluoromethyl)phenyl)cyclobutyl 3-((1,1-dioxidothietan-3-yl)carbamoyl)but-3-enoate;
1-(4-(trifluoromethyl)phenyl)cyclobutyl 3-((2-(methylsulfonyl)ethyl)carbamoyl)but-3-enoate;
(2-((3-(1-(4-((trifluoromethyl)thio)phenyl)cyclopropyl)-1,2,4-oxadiazol-5-yl)methyl)acryloyl)glycine;
3,3,3-trifluoro-2-(2-((3-(1-(4-((trifluoromethyl)thio)phenyl)cyclopropyl)-1,2,4-oxadiazol-5-yl)methyl)acrylamido)propanoic acid;
(2-methylene-4-oxo-4-(1-(4-(trifluoromethyl)phenyl)cyclobutoxy)butanoyl) glycine;
   or a pharmaceutically acceptable salt and/or solvate of any one thereof.

Clause 79. A pharmaceutical composition comprising a compound, pharmaceutically acceptable salt and/or solvate thereof according to any one of clauses 1 to 78.

Clause 80. A compound, pharmaceutically acceptable salt and/or solvate thereof according to any one of clauses 1 to 78 or a pharmaceutical composition according to clause 79 for use as a medicament.

Clause 81. A compound, pharmaceutically acceptable salt and/or solvate thereof according to any one of clauses 1 to 78 or a pharmaceutical composition according to clause 79 for use in treating or preventing an inflammatory disease or a disease associated with an undesirable immune response.

Clause 82. Use of a compound, pharmaceutically acceptable salt and/or solvate thereof according to any one of clauses 1 to 78 or a pharmaceutical composition according to clause 79 in the manufacture of a medicament for treating or preventing an inflammatory disease or a disease associated with an undesirable immune response.

Clause 83. A method of treating or preventing an inflammatory disease or a disease associated with an undesirable immune response, which comprises administering a compound, pharmaceutically acceptable salt and/or solvate thereof according to any one of clauses 1 to 78 or a pharmaceutical composition according to clause 79.

Clause 84. The compound, pharmaceutically acceptable salt and/or solvate thereof, pharmaceutical composition, compound for use, use or method according to any one of clauses 1 to 84, for treating an inflammatory disease or a disease associated with an undesirable immune response.

Clause 85. The compound, pharmaceutically acceptable salt and/or solvate thereof, pharmaceutical composition, compound for use, use or method according to any one of clauses 1 to 84, for preventing an inflammatory disease or a disease associated with an undesirable immune response.

Clause 86. The compound, pharmaceutically acceptable salt and/or solvate thereof, pharmaceutical composition, compound for use, use or method according to any one of clauses 1 to 84, for treating or preventing an inflammatory disease.

Clause 87. The compound, pharmaceutically acceptable salt and/or solvate thereof, pharmaceutical composition, compound for use, use or method according to any one of clauses 1 to 84, for treating or preventing a disease associated with an undesirable immune response.

Clause 88. The compound, pharmaceutically acceptable salt and/or solvate thereof, pharmaceutical composition, compound for use, use or method according to any one of clauses 1 to 87, wherein the inflammatory disease or disease associated with an undesirable immune response is, or is associated with, a disease selected from the group consisting of: psoriasis (including chronic plaque, erythrodermic, pustular, guttate, inverse and nail variants), asthma, chronic obstructive pulmonary disease (COPD, including chronic bronchitis and emphysema), heart failure (including left ventricular failure), myocardial infarction, angina pectoris, other atherosclerosis and/or atherothrombosis-related disorders (including peripheral vascular disease and ischaemic stroke), a mitochondrial and neurodegenerative disease (such as Parkinson's disease, Alzheimer's disease, Huntington's disease, amyotrophic lateral sclerosis, retinitis pigmentosa or mitochondrial encephalomyopathy), autoimmune paraneoplastic retinopathy, transplantation rejection (including antibody-mediated and T cell-mediated forms), multiple sclerosis, transverse myelitis, ischaemia-reperfusion injury (e.g. during elective surgery such as cardiopulmonary bypass for coronary artery bypass grafting or other cardiac surgery, following percutaneous coronary intervention, following treatment of acute ST-elevation myocardial infarction or ischaemic stroke, organ transplantation, or acute compartment syndrome), AGE-induced genome damage, an inflammatory bowel disease (e.g. Crohn's disease or ulcerative colitis), primary sclerosing cholangitis (PSC), PSC-autoimmune hepatitis overlap syndrome, non-alcoholic fatty liver disease (non-alcoholic steatohepatitis), rheumatica, granuloma annulare, cutaneous lupus erythematosus (CLE), systemic lupus erythematosus (SLE), lupus nephritis, drug-induced lupus, autoimmune myocarditis or myopericarditis, Dressler's syndrome, giant cell myocarditis, post-pericardiotomy syndrome, drug-induced hypersensitivity syndromes (including hypersensitivity myocarditis), eczema, sarcoidosis, erythema nodosum, acute disseminated encephalomyelitis (ADEM), neuromyelitis optica spectrum disorders, MOG (myelin oligodendrocyte glycoprotein) antibody-associated disorders (including MOG-EM), optic neuritis, CLIPPERS (chronic lymphocytic inflammation with pontine perivascular enhancement responsive to steroids), diffuse myelinoclastic sclerosis, Addison's disease, alopecia areata, ankylosing spondylitis, other spondyloarthritides (including peripheral spondyloarthritis, that is associated with psoriasis, inflammatory bowel disease, reactive arthritis or juvenile onset forms), antiphospholipid antibody syndrome, autoimmune hemolytic anaemia, autoimmune hepatitis, autoimmune inner ear disease, pemphigoid (including bullous pemphigoid, mucous membrane pemphigoid, cicatricial pemphigoid, herpes gestationis or pemphigoid gestationis, ocular cicatricial pemphigoid), linear IgA disease, Behçet's disease, celiac disease, Chagas disease, dermatomyositis, diabetes mellitus type I, endometriosis, Goodpasture's syndrome, Graves' disease, Guillain-Barre syndrome and its subtypes (including acute inflammatory demyelinating polyneuropathy, AIDP, acute motor axonal neuropathy (AMAN), acute motor and sensory axonal neuropathy (AMSAN), pharyngeal-cervical-brachial variant, Miller-Fisher variant and Bickerstaff's brainstem encephalitis), progressive inflammatory neuropathy, Hashimoto's disease, hidradenitis suppurativa, inclusion body myositis, necrotising myopathy, Kawasaki disease, IgA nephropathy, Henoch-Schonlein purpura, idiopathic thrombocytopenic purpura, thrombotic thrombocytopenic purpura (TTP), Evans' syndrome, interstitial cystitis, mixed connective tissue disease, undifferentiated connective tissue disease, morphea, myasthenia gravis (including MuSK antibody positive and seronegative variants), narcolepsy, neuromyotonia, pemphigus vulgaris, pernicious anaemia, psoriatic arthritis, polymyositis, primary biliary cholangitis (also known as primary biliary cirrhosis), rheumatoid arthritis, palindromic rheumatism, schizophrenia, autoimmune (meningo-)encephalitis syndromes, scleroderma, Sjogren's syndrome, stiff person syndrome, polymylagia rheumatica, giant cell arteritis (temporal arteritis), Takayasu arteritis, polyarteritis nodosa, Kawasaki disease, granulomatosis with polyangitis (GPA; formerly known as Wegener's granulomatosis), eosinophilic granulomatosis with polyangiitis (EGPA; formerly known as Churg-Strauss syndrome), microscopic polyarteritis/polyangiitis, hypocomplementaemic urticarial vasculitis, hypersensitivity vasculitis, cryoglobulinemia, thromboangiitis obliterans (Buerger's disease), vasculitis, leukocytoclastic vasculitis, vitiligo, acute disseminated encephalomyelitis, adrenoleukodystrophy, Alexander's disease, Alper's disease, balo concentric sclerosis or Marburg disease, cryptogenic organising pneumonia (formerly known as bronchiolitis obliterans organizing pneumonia), Canavan disease, central nervous system vasculitic syndrome, Charcot-Marie-Tooth disease, childhood ataxia with central nervous system hypomyelination, chronic inflammatory demyelinating polyneuropathy (CIDP), diabetic retinopathy, globoid cell leukodystrophy (Krabbe disease), graft-versus-host disease (GVHD) (including acute and chronic forms, as well as intestinal GVHD), hepatitis C (HCV) infection or complication, herpes simplex viral infection or complication, human immunodeficiency virus (HIV) infection or complication, lichen planus, monomelic amyotrophy, cystic fibrosis, pulmonary arterial hypertension (PAH, including idiopathic PAH), lung sarcoidosis, idiopathic pulmonary fibrosis, paediatric asthma, atopic dermatitis, allergic dermatitis, contact dermatitis, allergic rhinitis, rhinitis, sinusitis, conjunctivitis, allergic conjunctivitis, keratoconjunctivitis sicca, dry eye, xerophthalmia, glaucoma, macular oedema, diabetic macular oedema, central retinal vein occlusion (CRVO), macular degeneration (including dry and/or wet age related macular degeneration, AMD), post-operative cataract inflammation, uveitis (including posterior, anterior, intermediate and pan uveitis), iridocyclitis, scleritis, corneal graft and limbal cell transplant rejection, gluten sensitive enteropathy (coeliac disease), dermatitis herpetiformis, eosinophilic esophagitis, achalasia, autoimmune dysautonomia, autoimmune encephalomyelitis, autoimmune oophoritis, autoimmune orchitis, autoimmune pancreatitis, aortitis and periaortitis, autoimmune retinopathy, autoimmune urticaria, (idiopathic) Castleman's disease, Cogan's syndrome, IgG4-related disease, retroperitoneal fibrosis, juvenile idiopathic arthritis including systemic juvenile idiopathic arthritis (Still's disease), adult-onset Still's disease, ligneous conjunctivitis, Mooren's ulcer, pityriasis lichenoides et varioliformis acuta (PLEVA, also known as Mucha-Habermann disease), multifocal motor neuropathy (MMN), paediatric acute-onset neuropsychiatric syndrome (PANS) (including paediatric autoimmune neuropsychiatric disorders associated with streptococcal infections (PANDAS)), paraneoplastic syndromes (including paraneoplastic cerebellar degeneration, Lambert-Eaton myaesthenic syndrome, limbic encephalitis, brainstem encephalitis, opsoclonus myoclonus ataxia syndrome, anti-NMDA receptor encephalitis, thymoma-associated multiorgan autoimmunity), perivenous encephalomyelitis, reflex sympathetic dystrophy, relapsing polychondritis, sperm & testicular autoimmunity, Susac's syndrome, Tolosa-Hunt syndrome, Vogt-Koyanagi-Harada Disease, anti-synthetase syndrome, autoimmune enteropathy, immune dysregulation polyendocrinopathy enteropathy X-linked (IPEX), microscopic colitis, autoimmune lymphoproliferative syndrome (ALPS), autoimmune polyendocrinopathy-candidiasis-ectodermal dystrophy syndrome (APEX), gout, pseudogout, amyloid (including AA or secondary amyloidosis), eosinophilic fasciitis (Shulman syndrome) progesterone hypersensitivity (including progesterone dermatitis), amilial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutieres syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria), Schnitzler syndrome; familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders (e.g. myocardial infarction, angina, ischaemic heart failure, ischaemic nephropathy, ischaemic stroke, peripheral vascular disease, aortic aneurysm), and renal inflammatory disorders (e.g. diabetic nephropathy, membranous nephropathy, minimal change disease, crescentic glomerulonephritis, acute kidney injury, renal transplantation).

Clause 89. The compound, pharmaceutically acceptable salt and/or solvate thereof, pharmaceutical composition, compound for use, use or method according to clause 88, wherein the inflammatory disease or disease associated with an undesirable immune response is selected from the group consisting of rheumatoid arthritis, psoriatic arthritis, ankylosing spondylitis, systemic lupus erythematosus, multiple sclerosis, psoriasis, Crohn's disease, ulcerative colitis, uveitis, cryopyrin-associated periodic syndromes, Muckle-Wells syndrome, juvenile idiopathic arthritis, chronic obstructive pulmonary disease and asthma.

Clause 90. The compound, pharmaceutically acceptable salt and/or solvate thereof, pharmaceutical composition, compound for use, use or method according to clause 89, wherein the inflammatory disease or disease associated with an undesirable immune response is multiple sclerosis.

Clause 91. The compound, pharmaceutically acceptable salt and/or solvate thereof, pharmaceutical composition, compound for use, use or method according to clause 89, wherein the inflammatory disease or disease associated with an undesirable immune response is psoriasis.

Clause 92. The compound, pharmaceutically acceptable salt and/or solvate thereof, pharmaceutical composition, compound for use, use or method according to clause 89, wherein the inflammatory disease or disease associated with an undesirable immune response is asthma.

Clause 93. The compound, pharmaceutically acceptable salt and/or solvate thereof, pharmaceutical composition, compound for use, use or method according to clause 89, wherein the inflammatory disease or disease associated with an undesirable immune response is chronic obstructive pulmonary disease.

Clause 94. The compound, pharmaceutically acceptable salt and/or solvate thereof, pharmaceutical composition, compound for use, use or method according to clause 89, wherein the inflammatory disease or disease associated with an undesirable immune response is systemic lupus erythematosus.

Clause 95. The compound, pharmaceutically acceptable salt and/or solvate thereof, pharmaceutical composition, compound for use, use or method according to any one of clauses 1 to 94, wherein the compound is for administration to a human subject.

Clause 96. The compound, pharmaceutically acceptable salt and/or solvate thereof, pharmaceutical composition, compound for use, use or method according to any one of clauses 1 to 95, for use in combination with a further therapeutic agent, such as a corticosteroid (glucocorticoid), retinoid (e.g. acitretin, isotretinoin, tazarotene), anthralin, vitamin D analogue (e.g. cacitriol, calcipotriol), calcineurin inhibitors (e.g. tacrolimus, pimecrolimus), phototherapy or photochemotherapy (e.g. psoralen ultraviolet irradiation, PUVA) or other form of ultraviolet light irradiation therapy, ciclosporine, a thiopurine (e.g. azathioprine, 6-mercaptopurine), methotrexate, an anti-TNFα agents (e.g. infliximab, etanercept, adalimumab, certolizumab, golimumab or a biosimilar), phosphodiesterase-4 (PDE4) inhibition (e.g. apremilast, crisaborole), anti-IL-17 agent (e.g. brodalumab, ixekizumab, secukinumab), anti-IL12/IL-23 agent (e.g. ustekinumab, briakinumab), anti-IL-23 agent (e.g. guselkumab, tildrakizumab), JAK (Janus Kinase) inhibitor (e.g. tofacitinib, ruxolitinib, baricitinib, filgotinib, upadacitinib), plasma exchange, intravenous immune globulin (IVIG), cyclophosphamide, anti-CD20 B cell depleting agent (e.g. rituximab, ocrelizumab, ofatumumab, obinutuzumab), anthracycline analogue (e.g. mitoxantrone), cladribine, sphingosine 1-phosphate receptor modulator or sphingosine analogue (e.g. fingolimod, siponimod, ozanimod, etrasimod), interferon beta preparation (including interferon beta 1b/1a), glatiramer, anti-CD3 therapy (e.g. OKT3), anti-CD52 targeting agent (e.g. alemtuzumab), leflunomide, teriflunomide, gold compound, laquinimod, potassium channel blocker (e.g. dalfampridine/4-aminopyridine), mycophenolic acid, mycophenolate mofetil, purine analogue (e.g. pentostatin), mTOR (mechanistic target of rapamycin) pathway inhibitor (e.g. sirolimus, everolimus), anti-thymocyte globulin (ATG), IL-2 receptor (CD25) inhibitor (e.g. basiliximab, daclizumab), anti-IL-6 receptor or anti-IL-6 agent (e.g. tocilizumab, siltuximab), Bruton's tyrosine kinase (BTK) inhibitor (e.g. ibrutinib), tyrosine kinase inhibitor (e.g. imatinib), ursodeoxycholic acid, hydroxychloroquine, chloroquine, B cell activating factor (BAFF, also known as BLyS, B lymphocyte stimulator) inhibitor (e.g. belimumab, blisibimod), other B cell targeted therapy including a fusion protein targeting both APRIL (A PRoliferation-Inducing Ligand) and BLyS (e.g. atacicept), PI3K inhibitor including pan-inhibitor or one targeting the p110δ and/or p110γ containing isoforms (e.g. idelalisib, copanlisib, duvelisib), an interferon α receptor inhibitor (e.g. anifrolumab, sifalimumab), T cell co-stimulation blocker (e.g. abatacept, belatacept), thalidomide and its derivatives (e.g. lenalidomide), dapsone, clofazimine, a leukotriene antagonist (e.g. montelukast), theophylline, anti-IgE therapy (e.g. omalizumab), an anti-IL-5 agent (e.g. mepolizumab, reslizumab), a long-acting muscarinic agent (e.g. tiotropium, aclidinium, umeclidinium), a PDE4 inhibitor (e.g. roflumilast), riluzole, a free radical scavenger (e.g. edaravone), a proteasome inhibitor (e.g. bortezomib), a complement cascade inhibitor including one directed against C₅ (e.g. eculizumab), immunoadsor, antithymocyte globulin, 5-aminosalicylates and their derivatives (e.g. sulfasalazine, balsalazide, mesalamine), an anti-integrin agent including one targeting α4β1 and/or α4β7 integrins (e.g. natalizumab, vedolizumab), an anti-CD11-α agent (e.g. efalizumab), a non-steroidal anti-inflammatory drug (NSAID) including a salicylate (e.g. aspirin), a propionic acid (e.g. ibuprofen, naproxen), an acetic acid (e.g. indomethacin, diclofenac, etodolac), an oxicam (e.g. meloxicam) a fenamate (e.g. mefenamic acid), a selective or relatively selective COX-2 inhibitor (e.g. celecoxib, etroxicoxib, valdecoxib and etodolac, meloxicam, nabumetone), colchicine, an IL-4 receptor inhibitor (e.g. dupilumab), topical/contact immunotherapy (e.g. diphenylcyclopropenone, squaric acid dibutyl ester), anti-IL-1 receptor therapy (e.g. anakinra), IL-1β inhibitor (e.g. canakinumab), IL-1 neutralising therapy (e.g. rilonacept), chlorambucil, a specific antibiotic with immunomodulatory properties and/or ability to modulate NRF2 (e.g. tetracyclines including minocycline, clindamycin, macrolide antibiotics), anti-androgenic therapy (e.g. cyproterone, spironolactone, finasteride), pentoxifylline, ursodeoxycholic acid, obeticholic acid, fibrate, a cystic fibrosis transmembrane conductance regulator (CFTR) modulator, a VEGF (vascular endothelial growth factor) inhibitor (e.g. bevacizumab, ranibizumab, pegaptanib, aflibercept), pirfenidone or mizoribine.

Clause 97. A process for preparing a compound of formula (I) according to clause 1, or a salt such as a pharmaceutical acceptable salt thereof, which comprises reacting a compound of formula (II):

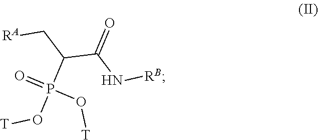

or a salt thereof;
with formaldehyde or a formaldehyde equivalent thereof, e.g., paraformaldehyde; wherein R^{A} and R^{B} are defined in clause 1, and T is C₁₋₄ alkyl such as ethyl.

Clause 98. A compound of formula (II):

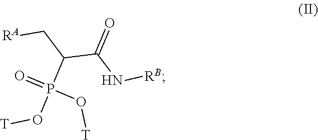

or a salt thereof;
wherein R^{A} and R^{B} are defined in clause 1, and T is C₁₋₄ alkyl such as ethyl.

Clause 99. A process for preparing a compound of formula (I) or a salt such as a pharmaceutically acceptable salt thereof, which comprises reacting a compound of formula (XV):

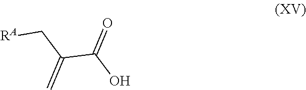

wherein R^{A} is as defined for formula (I);
with a halogenating agent to produce an acyl halide and reacting the acyl halide with a compound of formula (III):

H₂N—R^{B} (III)
wherein R^{B} is as defined in claim 1; or
reacting a compound of formula (XV) as defined above with a compound of formula (III) in the presence of a coupling agent and a base.

Clause 100. A compound of formula (XV):

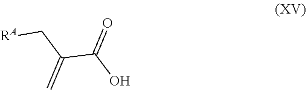

wherein R^{A} is as defined in clause 1.

### ABBREVIATIONS USED IN EXAMPLES

4OI 4-octyl itaconic acid
Ac acetyl
AcOH acetic acid
app. apparent
aq. aqueous
BBFO broadband fluorine observe
BEH ethylene bridged hybrid
br. broad
BTEAC benzyl(triethyl)ammonium chloride
ca. circa
CSH charged surface hybrid
d doublet
DAD diode array detector
DBU 1,8-diazabicyclo(5.4.0)undec-7-ene
DCC N,N′-Dicyclohexylcarbodiimide
DCM dichloromethane
DIPEA N,N-diisopropylethylamine
DMAP 4-(Dimethylamino)pyridine
DMF dimethyl fumarate
DMSO dimethyl sulfoxide
DMSO-d6 deuterated dimethyl sulfoxide
EDC/EDCI N-Ethyl-N′-(3-dimethylaminopropyl)carbodiimide hydrochloride
Et ethyl
ES⁺ electrospray
FBS fetal bovine serum
g gram(s)
GSH glutathione
h hour(s)
HATU 1-[bis(dimethylamino)methylene]-1H-1,2,3-triazolo[4,5-b]pyridinium 3-oxid hexafluorophosphate
HPLC high-performance liquid chromatography
LCMS liquid chromatography-mass spectrometry
LPS lipopolysaccharide
m multiplet
M molar concentration/molar mass
Me methyl
m/z mass to charge ratio
(M)Hz (mega)hertz
min(s) minute(s)
mL millilitres
mm millimetre
mmol millimole
MS mass spectrometry
MSD mass selective detector
MTBE methyl tertiary-butyl ether
N Normal
nm nanometre
NMP N-methyl-2-pyrrolidone
NMR nuclear magnetic resonance
NRF2 nuclear factor (erythroid-derived 2)-like 2
O/N overnight
PBS phosphate buffered saline
PDA photodiode array
ppm parts per million
prep preparatory
rpm revolutions per minute
RT room temperature
s singlet
S-Phos dicyclohexyl(2′,6′-dimethoxy[1,1′-biphenyl]-2-yl)phosphane
sat. saturated
t triplet
T3P propylphosphonic anhydride
TFA trifluoroacetic acid
THF tetrahydrofuran
μL microlitre
μM micromolar
UPLC ultra performance liquid chromatography
VWD variable wavelength detector
wt. weight
° C. degrees Celsius

### EXAMPLES

### Analytical Equipment

NMR spectra were recorded using a Bruker 400 MHz Avance Ill spectrometer fitted with a BBFO 5 mm probe, or a Bruker 500 MHz Avance Ill HD spectrometer equipped with a Bruker 5 mm SmartProbe™. Spectra were measured at 298 K, unless indicated otherwise, and were referenced relative to the solvent resonance. The chemical shifts are reported in parts per million. Data were acquired using Bruker TopSpin software.

UPLC/MS analysis was carried out on a Waters Acquity UPLC system using either a Waters Acquity CSH C18 or BEH C18 column (2.1×30 mm) maintained at a temperature of 40° C. and eluted with a linear acetonitrile gradient appropriate for the lipophilicity of the compound over 3 or 10 minutes at a constant flow rate of 0.77 mL/min. The aqueous portion of the mobile phase was either 0.1% Formic Acid (CSH C18 column) or 10 mM Ammonium Bicarbonate (BEH C18 column). LC-UV chromatograms were recorded using a Waters Acquity PDA detector between 210 and 400 nm. Mass spectra were recorded using a Waters Acquity Qda detector with electrospray ionisation switching between positive and negative ion mode. Sample concentration was adjusted to give adequate UV response.

LCMS analysis was carried out on an Agilent LCMS system using either a Waters Acquity CSH C18 or BEH C18 column (4.6×30 mm) maintained at a temperature of 40° C. and eluted with a linear acetonitrile gradient appropriate for the lipophilicity of the compound over 4 or 15 minutes at a constant flow rate of 2.5 mL/min. The aqueous portion of the mobile phase was either 0.1% Formic Acid (CSH C18 column) or 10 mM Ammonium Bicarbonate (BEH C18 column). LC-UV chromatograms were recorded using an Agilent VWD or DAD detector at 254 nm. Mass spectra were recorded using an Agilent MSD detector with electrospray ionisation switching between positive and negative ion mode. Sample concentration was adjusted to give adequate UV response.

### Commercial Materials

All starting materials disclosed herein are commercially available. Dimethyl itaconate was purchased from Sigma-Aldrich (product number: 109533). 4-Octyl itaconate was purchased from BOC biosciences (product number: B0001-007866). 2-(2-chlorobenzyl)acrylic acid was purchased from ChemSpace.

Unless otherwise stated all reactions were stirred. Organic solutions were routinely dried over anhydrous magnesium sulfate. Hydrogenations were performed on a Thales H-cube flow reactor under the conditions stated or under pressure in a gas autoclave (bomb).

### Intermediate 1—4-(tert-butoxy)-3-(diethoxyphosphoryl)-4-oxobutanoic acid

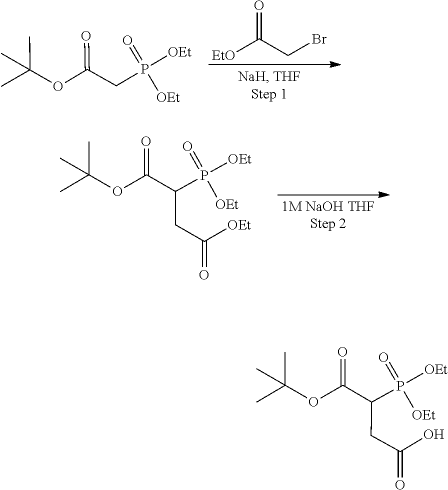

### Step 1

Sodium hydride (60% dispersion in mineral oil, 9.0 g, 225 mmol) was added portionwise to a solution of tert-butyl 2-(diethoxyphosphoryl)acetate (50 mL, 213 mmol) in THF (500 mL) at 0° C. The mixture was stirred for 15 min before ethyl bromoacetate (23 mL, 210 mmol) was added dropwise. The mixture was stirred for 1 h then quenched with sat. aq. ammonium chloride (100 mL) and extracted with EtOAc (3×100 mL). The combined organic phases were washed with brine (300 mL), dried (magnesium sulfate) and concentrated under reduced pressure to afford 1-(tert-butyl) 4-ethyl 2-(diethoxyphosphoryl)succinate (77.1 g, 182 mmol, 87% yield) as a colourless oil. LCMS m/z 361.2 (M+Na)+ (ES+). ¹H NMR (400 MHz, DMSO-d₆) δ 4.13-4.01 (m, 6H), 3.28 (ddd, J=23.8, 11.3, 3.9 Hz, 1H), 2.78 (ddd, J=17.2, 11.3, 8.2 Hz, 1H), 2.64 (ddd, J=17.1, 8.5, 4.0 Hz, 1H), 1.40 (s, 9H), 1.28-1.21 (m, 6H), 1.18 (t, J=7.1 Hz, 3H).

### Step 2

An aqueous solution of sodium hydroxide (1 M, 250 mL, 250 mmol) was added to a solution of 1-(tert-butyl) 4-ethyl 2-(diethoxyphosphoryl)succinate (77.1 g, 182 mmol) in THF (250 mL). The mixture was stirred at RT for 16 h. The mixture was partially concentrated under reduced pressure to ca. 250 mL, then extracted with EtOAc (3×100 mL). The aqueous phase was acidified to pH 1 with conc. hydrochloric acid and extracted with EtOAc (3×100 mL). The combined organic phases were washed with brine (250 mL), dried (magnesium sulfate) and concentrated under reduced pressure. The residue was triturated with hexane (300 mL) and the resulting solid collected by filtration to afford 4-(tert-butoxy)-3-(diethoxyphosphoryl)-4-oxobutanoic acid (53.0 g, 0.15 mol, 82% yield) as a white solid. LCMS m/z 333.2 (M+Na)+ (ES+). ¹H NMR (400 MHz, DMSO-d₆) δ 12.44 (s, 1H), 4.11-3.99 (m, 4H), 3.22 (ddd, J=23.7, 11.5, 3.7 Hz, 1H), 2.73 (ddd, J=17.3, 11.5, 7.6 Hz, 1H), 2.56 (ddd, J=17.3, 8.6, 3.7 Hz, 1H), 1.40 (s, 9H), 1.30-1.20 (m, 6H).

### Intermediate 2—2-(diethoxyphosphoryl)-3-(3-(1-(4-((trifluoromethyl)thio)phenyl) cyclopropyl)-1,2,4-oxadiazol-5-yl) propanoic acid

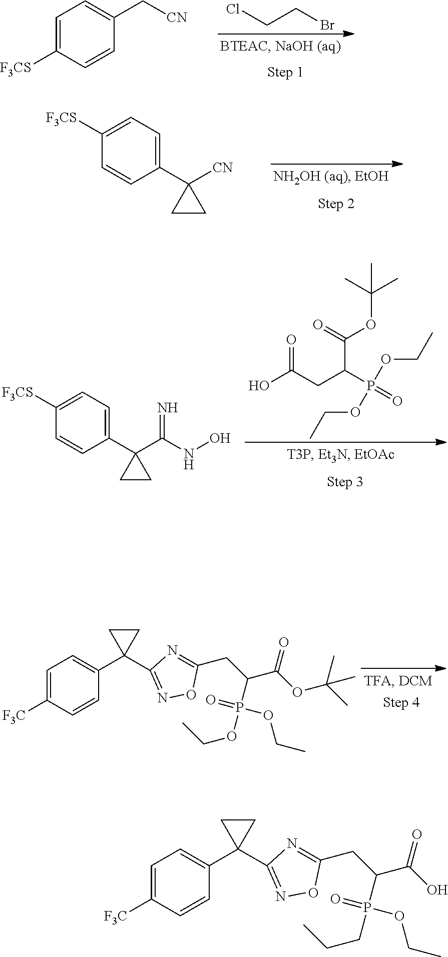

### Step 1

A solution of sodium hydroxide (27.6 g, 691 mmol) in water (40 mL) was added dropwise at 50° C. to a mixture of 2-(4-((trifluoromethyl)thio)phenyl)acetonitrile (25.0 g, 115 mmol), benzyl(triethyl)ammonium chloride (524 mg, 2.3 mmol) and 1-bromo-2-chloroethane (14.3 mL, 173 mmol). The mixture was stirred at 50° C. for 3 h, then cooled to RT. The mixture was diluted with water (300 mL) and extracted with DCM (3×75 mL). The combined organic phases were washed with 1 M hydrochloric acid (2×100 mL), water (100 mL), brine (100 mL), dried (magnesium sulfate) and concentrated under reduced pressure to afford 1-(4-((trifluoromethyl)thio)phenyl)cyclopropane-1-carbonitrile (28.6 g, 110 mmol, 96% yield) as an orange oil. ¹H NMR (400 MHz, DMSO-d₆) δ 7.84-7.66 (m, 2H), 7.59-7.39 (m, 2H), 1.92-1.78 (m, 2H), 1.69-1.55 (m, 2H).

### Step 2

Hydroxylamine (50% in water, 13 mL, 219 mmol) was added to a solution of 1-(4-((trifluoromethyl)thio)phenyl)cyclopropane-1-carbonitrile (28.6 g, 110 mmol) in EtOH (200 mL). The mixture was stirred at RT for 2 h, then heated to 45° C. and stirred for 16 h. The mixture was cooled to RT then concentrated under reduced pressure. The residue was co-evaporated with toluene (2×50 mL). The residue was recrystallised from MTBE/isohexane and the resulting solid was isolated by filtration, washing with isohexane (2×100 mL) to afford N-hydroxy-1-(4-((trifluoromethyl)thio)phenyl)cyclopropane-1-carboximidamide (20.4 g, 73 mmol, 66% yield) as a white solid. LCMS m/z 277.1 (M+H)⁺ (ES+). ¹H NMR (400 MHz, DMSO-d₆) δ 9.09 (s, 1H), 7.62 (d, J=8.3 Hz, 2H), 7.39 (d, J=8.4 Hz, 2H), 5.42 (s, 2H), 1.38-1.21 (m, 2H), 1.11-0.98 (m, 2H).

### Step 3

A solution of 2,4,6-tripropyl-1,3,5,2,4,6-trioxatriphosphinane 2,4,6-trioxide (T3P, 50% in EtOAc, 16.2 mL, 27 mmol) was added dropwise to a solution of N-hydroxy-1-(4-((trifluoromethyl)thio)phenyl)cyclopropane-1-carboximidamide (3.0 g, 10.9 mmol) and 4-(tert-butoxy)-3-(diethoxyphosphoryl)-4-oxobutanoic acid (Intermediate 1, 3.4 g, 10.9 mmol) and triethylamine (4.5 mL, 33 mmol) in EtOAc (5.5 mL) at RT. The mixture was heated to 80° C. for 16 h, then cooled to RT and poured into ice water (50 mL). The mixture was extracted with EtOAc (3×25 mL). The combined organic layers were washed with sat. aq. sodium bicarbonate (2×25 mL), brine (30 mL), dried (sodium sulfate) and concentrated under reduced pressure. The crude product was purified by chromatography on silica gel (0-100% MTBE/isohexane) to afford tert-butyl 2-(diethoxyphosphoryl)-3-(3-(1-(4-((trifluoromethyl)thio)phenyl)cyclopropyl)-1,2,4-oxadiazol-5-yl)propanoate (4.5 g, 8.1 mmol, 74% yield) as a yellow oil. LCMS m/z 573.3 (M+Na)⁺ (ES+). ¹H NMR (400 MHz, DMSO-d₆) δ 7.73-7.64 (m, 2H), 7.56-7.47 (m, 2H), 4.13-4.02 (m, 4H), 3.60-3.46 (m, 1H), 3.37-3.31 (m, 1H), 3.26-3.16 (m, 1H), 1.54-1.42 (m, 4H), 1.35 (s, 9H), 1.27-1.21 (m, 6H).

### Step 4

TFA (6.3 mL, 81 mmol) was added to a solution of tert-butyl 2-(diethoxyphosphoryl)-3-(3-(1-(4-((trifluoromethyl)thio)phenyl)cyclopropyl)-1,2,4-oxadiazol-5-yl)propanoate (4.5 g, 8.1 mmol) in DCM (30 mL) at RT. The mixture was stirred at RT for 18 h, then concentrated under reduced pressure to afford 2-(diethoxyphosphoryl)-3-(3-(1-(4-((trifluoromethyl)thio)phenyl)cyclopropyl)-1,2,4-oxadiazol-5-yl)propanoic acid (4.3 g, 8.1 mmol, quantitative yield) as a light yellow oil. LCMS m/z 495.2 (M+H)⁺ (ES+). ¹H NMR (400 MHz, DMSO-d₆) δ 12.86 (br. s, 1H), 7.71-7.66 (m, 2H), 7.55-7.50 (m, 2H), 4.11-4.02 (m, 4H), 3.55 (ddd, J=23.6, 10.1, 4.9 Hz, 1H), 3.42-3.30 (m, 1H), 3.22 (ddd, J=16.7, 9.9, 4.9 Hz, 1H), 1.52-1.43 (m, 4H), 1.25-1.20 (m, 6H).

### Intermediate 3—3-(3-(4-butylbenzyl)-1,2,4-oxadiazol-5-yl)-2-(diethoxyphosphoryl) propanoic acid

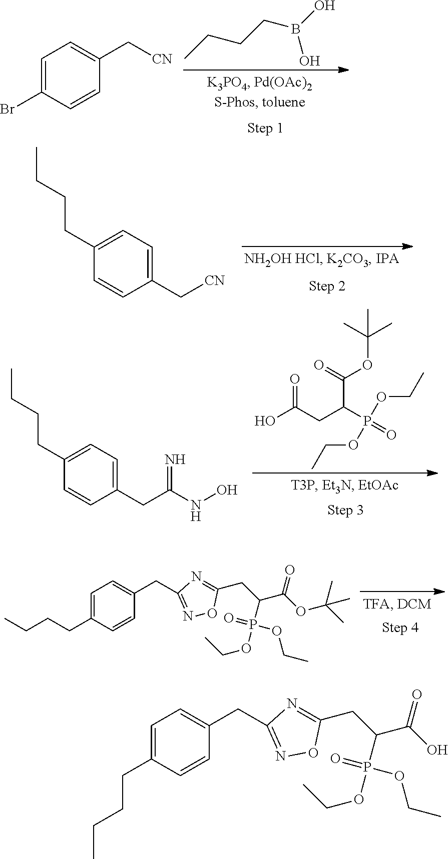

### Step 1

A mixture of 2-(4-bromophenyl)acetonitrile (15.0 g, 76.5 mmol), butylboronic acid (11.7 g, 114.8 mmol), palladium (II) acetate (1.7 g, 7.6 mmol), S-Phos (6.3 g, 15.3 mmol) and potassium phosphate (32.4 g, 153.0 mmol) in toluene (250 mL) was stirred at 110° C. overnight. The mixture was cooled to RT, filtered and the filtrate was concentrated under reduced pressure. The residue was purified by flash column chromatography on silica (0-20% ethyl acetate/petroleum ether) to give 2-(4-butylphenyl)acetonitrile (11.0 g, 63.5 mmol, 83% yield) as colorless liquid. ¹H NMR (400 MHz, CDCl₃) δ: 7.25-7.17 (m, 4H), 3.71 (s, 2H), 2.60 (t, J=7.6 Hz, 2H), 1.63-1.54 (m, 2H), 1.37-1.31 (m, 2H), 0.92 (t, J=7.2 Hz, 3H).

### Step 2

A mixture of hydroxylamine hydrochloride (1.2 g, 17.3 mmol) and sodium bicarbonate (1.5 g, 17.3 mmol) in isopropyl alcohol (23 mL) was stirred at room temperature for 20 min and 2-(4-butylphenyl)acetonitrile (2.0 g, 11.6 mmol) was added. The resulting suspension was stirred at 60° C. overnight. The mixture was cooled to room temperature, the solid filtered off and the filtrate concentrated under reduced pressure. The residue was redissolved in DCM (30 mL), the solid filtered off and the filtrate concentrated under reduced pressure to give 2-(4-butylphenyl)-N-hydroxyacetimidamide (2.5 g, 17.3 mmol, quantitative yield) as off-white solid. LCMS m/z 207.3 (M+H)⁺ (ES+).

### Step 3

2,4,6-tripropyl-1,3,5,2,4,6-trioxatriphosphinane 2,4,6-trioxide (T3P, 50% in ethyl acetate, 7.7 g, 24.3 mmol) was added at 0° C. to the solution of 2-(4-butylphenyl)-N-hydroxyacetimidamide (2.5 g, 12.1 mmol), 4-tert-butoxy-3-(diethoxyphosphoryl)-4-oxobutanoic acid (2.6 g, 8.5 mmol) and triethylamine (3.7 g, 36.4 mmol) in ethyl acetate (20 mL), and the mixture was heated to 80° C. and stirred overnight. The mixture was quenched with 0.5 N hydrochloric acid (40 mL), the organic layer separated and the aqueous layer extracted with ethyl acetate (2×50 mL). The combined organic layers were washed with water (2×25 mL) and brine, dried (sodium sulfate) and concentrated under reduced pressure. The residue was purified by flash column chromatography on silica (20-40% ethyl acetate/petroleum ether) to give tert-butyl 3-(3-(4-butylbenzyl)-1,2,4-oxadiazol-5-yl)-2-(diethoxyphosphoryl)propanoate (2.5 g, 3.6 mmol, 43% yield) as a colorless oil. LCMS m/z 481.2 (M+H)⁺ (ES+).

### Step 4

A solution of tert-butyl 3-(3-(4-butylbenzyl)-1,2,4-oxadiazol-5-yl)-2-(diethoxyphosphoryl)propanoate (2.5 g, 5.2 mmol) in TFA (6 mL) and DCM (12 mL) was stirred at room temperature overnight. The mixture was concentrated under reduced pressure and the residue was purified by reverse phase preparative HPLC (Column: Boston ODS 330 g Flash; Flow Rate: 60 mL/min; solvent system: MeCN/(10 mmol/L formic acid/water); MeCN gradient:60-80%; collection wavelength: 214 nm). Fractions were concentrated under reduced pressure to remove MeCN, and the residue was lyophilized to give 3-(3-(4-butylbenzyl)-1,2,4-oxadiazol-5-yl)-2-(diethoxyphosphoryl)propanoic acid (2.9 g, 5.2 mmol, quantitative yield) as yellow oil. LCMS m/z 425.1 (M+H)⁺ (ES+).

### Intermediate 4—2-(diethoxyphosphoryl)-4-((2-methyloctan-2-yl)oxy)-4-oxobutanoic acid

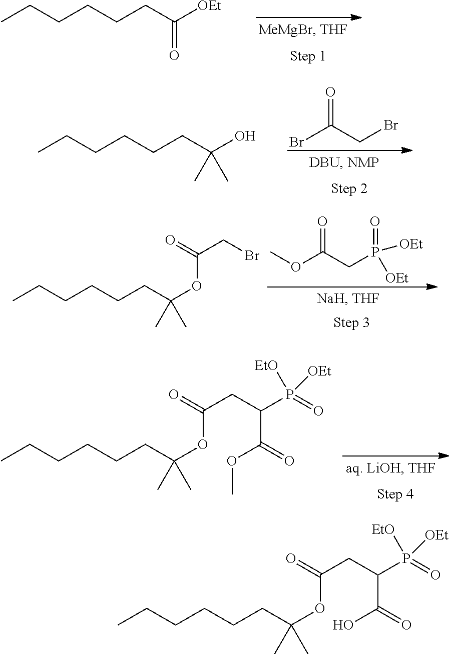

### Step 1

Methyl magnesium bromide solution (3 M in diethylether, 122 mL, 366 mmol,) was dropwise added at 0° C. to a solution of ethyl heptanoate (23.0 g, 145.3 mmol) in THF (250 mL) and the mixture was stirred at RT for 2 h. The reaction mixture was quenched with saturated aqueous ammonium chloride (120 mL), the organic layer separated and the aqueous layer extracted with tert-butyl methyl ether (2×150 mL). The combined organic layers were washed by brine, dried (sodium sulfate) and concentrated under reduced pressure to give 2-methyloctan-2-ol (20.0 g, 138.6 mmol, 95% yield) as colorless liquid. ¹H NMR (400 MHz, CDCl₃) δ: 1.48-1.45 (m, 2H), 1.35-1.29 (m, 8H), 1.20 (s, 6H), 0.88 (t, J=6.8 Hz, 3H).

### Step 2

2-bromoacetyl bromide (12.59 g, 62.39 mmol) was dropwise added at 0° C. to a solution of 2-methyloctan-2-ol (20.0 g, 138.6 mmol) and DBU (31.6 g, 208.0 mmol) in 1-methyl-2-pyrrolidinone (250 mL) and the mixture was stirred at RT for 16 hours. The reaction mixture was diluted with water (200 mL) and tert-butyl methyl ether (300 mL), The organic layer was separated and the aqueous layer was extracted with tert-butyl methyl ether (2×100 mL). The combined organic layers were washed by brine, dried (sodium sulfate) and concentrated under reduced pressure. The residue was purified by flash column chromatography (0-6% tert-butyl methyl ether/petroleum) to give 2-methyloctan-2-yl 2-bromoacetate (27.0 g, 45 mmol, 73% yield) as colorless oil. ¹H NMR (400 MHz, CDCl₃) δ: 3.75 (s, 2H), 1.77-1.73 (m, 2H), 1.45 (s, 6H), 1.28-1.23 (m, 8H), 0.90-0.84 (m, 3H).

### Step 3

NaH (60% in mineral oil, 4.5 g, 112.0 mmol,) was added portionwise at 0° C. to a solution of methyl 2-(diethoxyphosphoryl)acetate (21.4 g, 101.8 mmol) in THF (200 mL), and the reaction mixture was stirred at 0° C. for 0.5 h. A solution of 2-methyloctan-2-yl 2-bromoacetate (27.0 g, 101.8 mmol) in THF (100 mL) was added and the reaction mixture was stirred at RT overnight. The reaction mixture was quenched with saturated aqueous ammonium chloride, the organic layer separated and the aqueous layer extracted with EtOAc (2×150 mL). The combined organic layers were washed by brine, dried (sodium sulfate) and concentrated under reduced pressure to give 1-methyl 4-(2-methyloctan-2-yl) 2-(diethoxyphosphoryl)succinate (41.0 g, 101.8 mmol, quantitative yield) as a colorless oil. LCMS m/z 417.2 (M+Na)⁺ (ES+).

### Step 4

To a solution of 1-methyl 4-(2-methyloctan-2-yl) 2-(diethoxyphosphoryl)succinate (500 mg, 1.3 mmol) in THF (6 mL) was added 2 N lithium hydroxide (1.9 mL, 3.8 mmol), and the reaction mixture was stirred at RT for 5 h then left at 0° C. for 16 h. The mixture was acidified with 0.5 N hydrochloric acid until pH 3 and extracted with MTBE (3×6 mL). The combined organic layers were washed by brine, dried (sodium sulfate) and concentrated under reduced pressure. The residue was purified by flash column chromatography on silica (0-6% tert-butyl methyl ether/petroleum ether) to give 2-(diethoxyphosphoryl)-4-(2-methyloctan-2-yloxy)-4-oxobutanoic acid (460 mg, 1.2 mmol, 95% yield) as light yellow oil. LCMS m/z 403.2 (M+Na)⁺ (ES+).

### Intermediate 5—2-methylene-4-oxo-4-(1-(4-(trifluoromethyl)phenyl)cyclobutoxy)butanoic acid

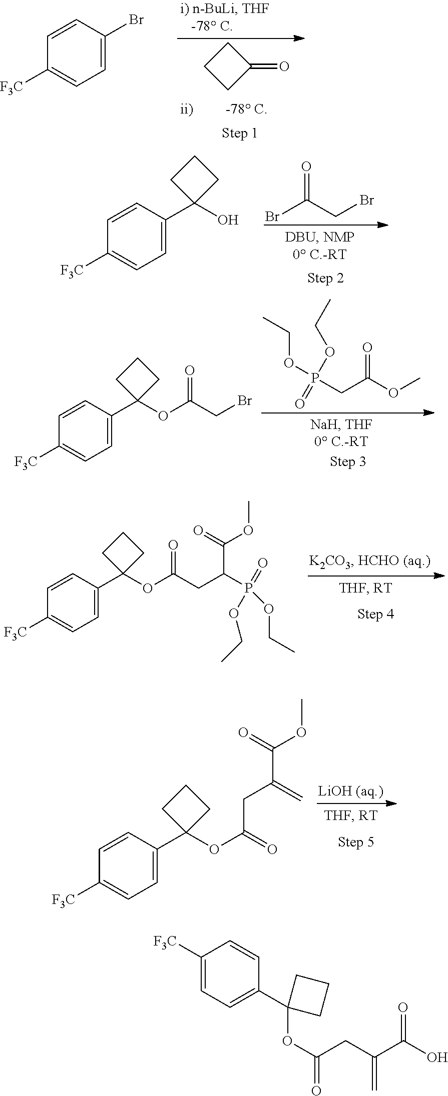

### Step 1

To a solution of 1-bromo-4-(trifluoromethyl)benzene (22.3 g, 99.5 mmol) in THF (180 mL) at −78° C. was added n-BuLi solution in hexane (2.5 M, 43.7 mL, 109.2 mmol) and the mixture was stirred at −78° C. for 1 h. Cyclobutanone (7.6 g, 109.2 mmol) was added, and the mixture was stirred at −78° C. for 5 h, then quenched with saturated aqueous NH₄Cl solution (200 mL). The phases were separated and the aqueous layer was extracted with MTBE (2×80 mL). The combined organic layers were washed with brine, dried over Na₂SO₄, filtered and concentrated under reduced pressure at 30° C., and the residue was purified by flash column chromatography (120 g silica, 0-14% MTBE/petroleum ether) to give 1-(4-(trifluoromethyl)phenyl)cyclobutan-1-ol (16.5 g, 76.3 mmol, 77% yield) as a yellow oil. ¹H NMR (400 MHz, CDCl₃) δ: 7.61 (s, 4H), 2.59-2.48 (m, 2H), 2.43-2.32 (m, 2H), 2.12-1.98 (m, 1H), 1.81-1.66 (m, 1H). One exchangeable proton not observed.

### Step 2

To a solution of 1-(4-(trifluoromethyl)phenyl)cyclobutan-1-ol (16.5 g, 76.3 mmol) and DBU (23.2 g, 153.4 mmol) in 1-methyl-2-pyrrolidinone (300 mL) at 0° C. was slowly added 2-bromoacetyl bromide (30.9 g, 153.4 mmol) dropwise, and the mixture was stirred at room temperature for 16 h. The reaction mixture was diluted with water (160 mL) and MTBE (200 mL), the phases were separated, and the aqueous layer was extracted with MTBE (2×150 mL). The combined organic layers were washed with brine, dried over Na₂SO₄, filtered and concentrated under reduced pressure at 40° C. The residue was purified by flash column chromatography (40 g silica, 0-10% MTBE/petroleum ether) to give 1-(4-(trifluoromethyl)phenyl)cyclobutyl 2-bromoacetate (20 g, 59.3 mmol, 77% yield) as a yellow oil. ¹H NMR (400 MHz, CDCl₃) δ: 7.67-7.57 (m, 4H), 3.76 (s, 2H), 2.75-2.60 (m, 4H), 2.11-1.98 (m, 1H), 1.85-1.70 (m, 1H).

### Step 3

To a solution of methyl 2-(diethoxyphosphoryl)acetate (8.1 g, 38.3 mmol) in THF (150 mL) at 0° C. was added NaH suspension in mineral oil (60 wt. %, 1.5 g, 38.3 mmol) and the reaction mixture was stirred at 0° C. for 0.5 h. 1-(4-(Trifluoromethyl)phenyl)cyclobutyl 2-bromoacetate (10 g, 29.8 mmol) was then added and the reaction mixture was stirred at room temperature overnight. The reaction mixture was quenched with dilute aqueous HCl (0.5 M) and adjusted to pH=5. The phases were separated, and the aqueous layer was extracted with EtOAc (2×100 mL). The combined organic layers were washed with brine, dried over Na₂SO₄, filtered and concentrated under reduced pressure at 40° C. to give 1-methyl 4-(1-(4-(trifluoromethyl)phenyl)cyclobutyl) 2-(diethoxyphosphoryl)succinate (14.4 g, 30.9 mmol, quantitative yield) as a colorless oil, which was used directly in the next step. LCMS (System 2, Method C) m/z 489.0 (M+Na)⁺ (ES⁺).

### Step 4

To a mixture of 1-methyl 4-(1-(4-(trifluoromethyl)phenyl)cyclobutyl) 2-(diethoxyphosphoryl)succinate (14.4 g, 30.9 mmol) and potassium carbonate (8.5 g, 61.4 mmol) in THF (200 mL) at room temperature was added formaldehyde solution in water (37 wt. %, 16.3 mL, 153.6 mmol) and the reaction mixture was stirred at room temperature for 4 h. The reaction mixture was diluted with H₂O (150 mL) and extracted with MTBE (2×200 mL). The combined organic layers were washed with H₂O (2×100 mL) and brine, dried over Na₂SO₄, filtered and concentrated under reduced pressure at 30° C. The residue was purified by flash column chromatography (120 g silica, 0-10% MTBE/petroleum ether) to give 1-methyl 4-(1-(4-(trifluoromethyl)phenyl)cyclobutyl) 2-methylenesuccinate (6 g, 17.5 mmol, 56% yield) as a colorless oil. LCMS (System 2, Method C) m/z 365.0 (M+Na)⁺ (ES⁺).

### Step 5

To a solution of 1-methyl 4-(1-(4-(trifluoromethyl)phenyl)cyclobutyl) 2-methylenesuccinate (6.0 g, 17.6 mmol) in THF (30 mL) was added LiOH solution in water (2 M, 13.5 mL, 26.3 mmol), and the reaction mixture was stirred at room temperature for 6 h and then kept in a fridge at 0° C. overnight. The reaction mixture was acidified with dilute aqueous HCl (0.5 M) until pH=3 and extracted with EtOAc (2×10 mL). The combined EtOAc layers were washed with brine, dried over Na₂SO₄, filtered and concentrated under reduced pressure at 30° C. to give a residue comprising of a 2:1 mixture of 2-methylene-4-oxo-4-(1-(4-(trifluoromethyl)phenyl)cyclobutoxy)butanoic acid and 2-methyl-4-oxo-4-(1-(4-(trifluoromethyl)phenyl)cyclobutoxy)but-2-enoic acid. The mixture was purified by reversed phase column chromatography (330 g C18 silica; flow rate: 60 mL/min; 60-85% MeCN/(10 mM formic acid/water); collection wavelength: 214 nm). The collected fractions were concentrated under reduced pressure to remove MeCN, and the residue was lyophilized to give impure 2-methylene-4-oxo-4-(1-(4-(trifluoromethyl)phenyl)cyclobutoxy) butanoic acid (1.8 g, 5.48 mmol, 31% yield) as a white solid. A small quantity (100 mg) was further purified by preparative HPLC (Column: Waters X-Bridge C18 OBD 10 μm 19×250 mm; Flow Rate: 20 mL/min; solvent system: MeCN/(0.2% formic acid/water); gradient: 55-95% MeCN; collection wavelength: 214 nm). The collected fractions were concentrated under reduced pressure at 30° C. to remove MeCN, and the residue was lyophilized to give 2-methylene-4-oxo-4-(1-(4-(trifluoromethyl)phenyl)cyclobutoxy)butanoic acid (47 mg) as a white solid. LCMS m/z 351.0 (M+Na)⁺ (ES⁺). ¹H NMR (400 MHz, DMSO-d6) δ: 12.65 (br, 1H), 7.71 (d, J=8.5 Hz, 2H), 7.65 (d, J=8.2 Hz, 2H), 6.13 (d, J=1.6 Hz, 1H), 5.74 (d, J=1.5 Hz, 1H), 3.33 (d, J=3.9 Hz, 2H), 2.61-2.50 (m, 4H), 2.02-1.89 (m, 1H), 1.84-1.69 (m, 1H).

### Intermediate 6—(R)-2-methylene-4-oxo-4-(1-(4-(trifluoromethyl)phenyl)ethoxy)butanoic acid

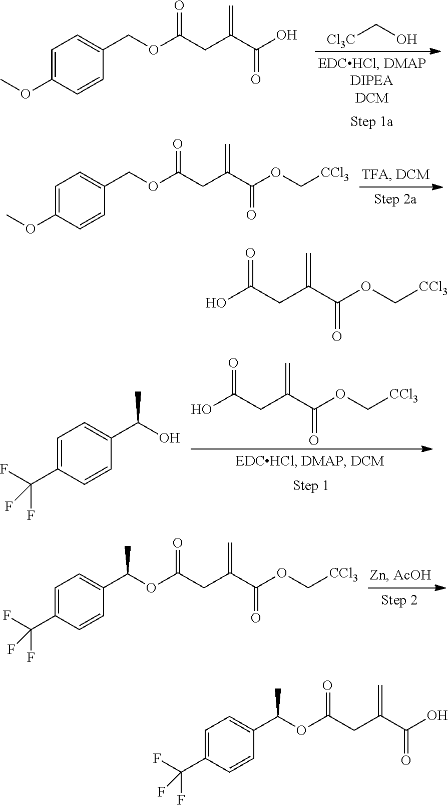

### Step 1a

To a solution of 4-((4-methoxybenzyl)oxy)-2-methylene-4-oxobutanoic acid (30.0 g, 120 mmol), 2,2,2-trichloroethan-1-ol (19.7 g, 132 mmol), DMAP (11.7 g, 96 mmol) and DIPEA (46.4 g, 360 mmol) in DCM (500 mL) at 0° C. was added EDC HCl (34.6 g, 180 mmol), and the resulting pale-yellow mixture was stirred at room temperature overnight. The mixture was quenched with dilute aqueous HCl (0.5 M), the phases were separated and the aqueous layer was extracted with DCM (3×500 mL). The combined organic phases were washed with brine, dried over Na₂SO₄ and filtered. The filtrate was concentrated under reduced pressure, and the residue was purified by flash column chromatography (120 g silica, 0-20% MTBE/petroleum ether) to give 4-(4-methoxybenzyl) 1-(2,2,2-trichloroethyl) 2-methylenesuccinate (35.0 g, 91.7 mmol, 76% yield) as a colorless oil. LCMS: m/z 402.8/404.8 (M+Na)⁺ (ES⁺).

### Step 2a

A solution of 4-(4-methoxybenzyl) 1-(2,2,2-trichloroethyl) 2-methylenesuccinate (35.0 g, 91.7 mmol) in TFA (40 mL) and DCM (80 mL) was stirred at room temperature for 16 h. The mixture was concentrated under reduced pressure and the residue was purified by reversed phase column chromatography (330 g C18 silica; flow rate: 60 mL/min; 60-80% MeCN/10 mM formic acid/water; collection wavelength: 214 nm). The collected fractions were concentrated under reduced pressure at 30° C. to remove MeCN, and the residue was lyophilized to give 3-((2,2,2-trichloroethoxy)carbonyl)but-3-enoic acid (23.0 g, 88.0 mmol, 96% yield) as a colorless oil. LCMS: m/z 282.8/284.8 (M+Na)⁺ (ES⁺).

### Step 1

To a solution of (R)-1-(4-(trifluoromethyl)phenyl)ethan-1-ol (200 mg, 1.05 mmol), 3-((2,2,2-trichloroethoxy)carbonyl)but-3-enoic acid (273 mg, 1.05 mmol) and DMAP (102 mg, 0.84 mmol) in DCM (4 mL) at 0° C. was added EDC HCl (303 mg, 1.58 mmol), and the resulting pale-yellow mixture was stirred at room temperature for 20 min. The mixture was quenched with dilute aqueous HCl (0.5 M), separated and the aqueous phase was extracted with DCM (3×5 mL). The combined organic layers were washed with brine, dried over Na₂SO₄ and filtered. The filtrate was concentrated under reduced pressure at 25° C., and the residue was purified by flash column chromatography (20 g silica, 0-20% MTBE/petroleum ether) to give (R)-1-(2,2,2-trichloroethyl) 4-(1-(4-(trifluoromethyl)phenyl)ethyl) 2-methylenesuccinate (200 mg, 0.46 mmol, 44% yield) as a pale-yellow oil. LCMS: m/z 454.8/456.8 (M+Na)⁺ (ES⁺).

### Step 2

A mixture of (R)-1-(2,2,2-trichloroethyl) 4-(1-(4-(trifluoromethyl)phenyl)ethyl) 2-methylenesuccinate (200 mg, 0.46 mmol) and zinc powder (150 mg, 2.32 mmol) in AcOH (2 mL) was stirred at room temperature for 2 days. The reaction mixture was filtered, and the filtrate was quenched with H₂O (3 mL), the phases were separated, and the aqueous layer was extracted with EtOAc (2×5 mL). The combined organic layers were washed with brine, dried over Na₂SO₄ and filtered. The filtrate was concentrated under reduced pressure and the residue was purified by preparative HPLC (Column: Waters Sunfire Prep C18 OBD 10 μm 19×250 mm; Flow Rate: 20 mL/min; solvent system: MeCN/(0.2% formic acid/water); gradient: 45-95% MeCN; collection wavelength: 214 nm). The collected fractions were concentrated under reduced pressure to remove MeCN, and the residue was lyophilized to give (R)-2-methylene-4-oxo-4-(1-(4-(trifluoromethyl)phenyl)ethoxy)butanoic acid (99 mg, 0.33 mmol, 71% yield) as a colorless oil. LCMS m/z 324.9 (M+Na)⁺ (ES⁺). ¹H NMR (400 MHz, DMSO-d6) δ: 12.64 (br, 1H), 7.72 (d, J=8.1 Hz, 2H), 7.58 (d, J=8.1 Hz, 2H), 6.16 (d, J=1.6 Hz, 1H), 5.87 (q, J=6.6 Hz, 1H), 5.78 (d, J=1.5 Hz, 1H), 3.38 (s, 2H), 1.46 (d, J=6.6 Hz, 3H).

### Intermediate 7—(S)-2-methylene-4-oxo-4-(1-(4-(trifluoromethyl)phenyl)ethoxy)butanoic acid

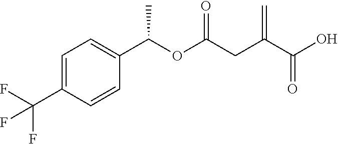 [SMILES: C=C(CC(=O)O[C@@H](C)c1ccc(C(F)(F)F)cc1)C(=O)O]

Prepared by an analogous method to Intermediate 6 starting from (S)-1-(4-(trifluoromethyl)phenyl)ethan-1-ol (200 mg, 1.05 mmol). Yield: 79 mg, 0.26 mmol. Colorless oil. LCMS m/z 324.9 (M+Na)⁺ (ES⁺). ¹H NMR (400 MHz, DMSO-d6) δ: 12.67 (br, 1H), 7.72 (d, J=8.1 Hz, 2H), 7.58 (d, J=8.1 Hz, 2H), 6.16 (d, J=1.6 Hz, 1H), 5.87 (q, J=6.6 Hz, 1H), 5.78 (d, J=1.5 Hz, 1H), 3.38 (s, 2H), 1.46 (d, J=6.6 Hz, 3H).

### Intermediate 8—4-(1-(3-fluoro-4-(trifluoromethyl)phenyl)cyclobutoxy)-2-methylene-4-oxobutanoic acid

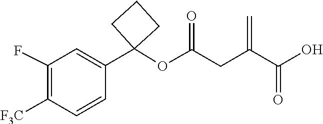 [SMILES: C=C(CC(=O)OC1(c2ccc(C(F)(F)F)c(F)c2)CCC1)C(=O)O]

Prepared by an analogous method to Intermediate 5 starting from 4-bromo-2-fluoro-1-(trifluoromethyl)benzene (7.0 g, 28.8 mmol). Yield: 122 mg, 0.35 mmol. White solid. LCMS m/z 369.0 (M+Na)⁺ (ES⁺). ¹H NMR (400 MHz, DMSO-d6) δ: 12.69 (br, 1H), 7.76 (t, J=7.9 Hz, 1H), 7.52 (d, J=12.2 Hz, 1H), 7.47 (d, J=8.2 Hz, 1H), 6.14 (s, 1H), 5.77 (s, 1H), 3.35 (s, 2H), 2.62-2.44 (m, 4H), 2.03-1.89 (m, 1H), 1.88-1.72 (m, 1H).

### Intermediate 9—2-methylene-4-oxo-4-((3-(4-(trifluoromethyl)phenyl)oxetan-3-yl)oxy)butanoic acid

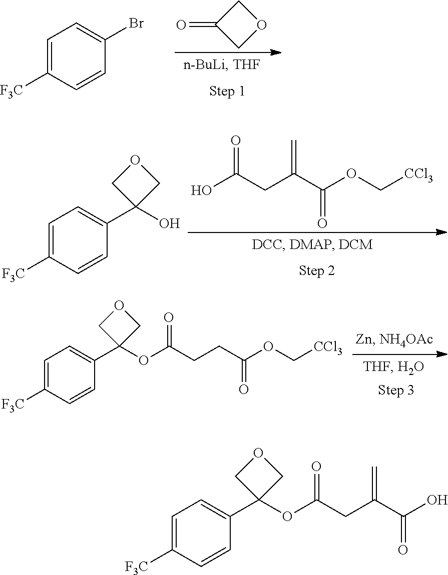

To the solution of 1-bromo-4-(trifluoromethyl)benzene (1.25 g, 5.56 mmol) in THF (15 mL) at −70° C. was added n-BuLi solution in hexane (2.5 M, 2.2 mL, 5.56 mmol) and the mixture was stirred at −70° C. for 1 h. Oxetan-3-one (400 mg, 5.56 mmol) was then added at −70° C., and the mixture was stirred at room temperature for 1 h. The reaction mixture was quenched with dilute aqueous HCl (0.5 M, 10 mL), the phases were separated and the aqueous layer was extracted with MTBE (2×20 mL). The combined organic layers were washed with brine, dried over Na₂SO₄, filtered and concentrated under reduced pressure, and the residue was purified by flash column chromatography (20 g silica, 0-25% MTBE/petroleum ether) to give 3-(4-(trifluoromethyl)phenyl)oxetan-3-ol (700 mg, 3.21 mmol, 58% yield) as a yellow oil. ¹H NMR (400 MHz, CDCl₃) δ: 7.78 (d, J=8.1 Hz, 2H), 7.69 (d, J=8.0 Hz, 2H), 4.95 (d, J=7.6 Hz, 2H), 4.88 (d, J=7.6 Hz, 2H), 2.63 (s, 1H).

### Step 2

A mixture of 3-(4-(trifluoromethyl)phenyl)oxetan-3-ol (700 mg, 3.21 mmol) 3-((2,2,2-trichloroethoxy)carbonyl)but-3-enoic acid (838 mg, 3.21 mmol), DCC (992 mg, 4.81 mmol) and DMAP (39 mg, 0.32 mmol) in DCM (10 mL) was stirred at room temperature for 30 minutes. The mixture was filtered, the filtrate was concentrated under reduced pressure, and the residue was purified by flash column chromatography (0-20% tert-butyl methyl ether/petroleum ether) to give 1-(2,2,2-trichloroethyl) 4-(3-(4-(trifluoromethyl)phenyl)oxetan-3-yl) 2-methylenesuccinate (900 mg, 61% yield) as a colorless oil. LCMS m/z 460.8 (M+H)⁺ (ES⁺).

### Step 3

To a mixture of 1-(2,2,2-trichloroethyl) 4-(3-(4-(trifluoromethyl)phenyl)oxetan-3-yl) 2-methylenesuccinate (900 mg, 1.95 mmol) and NH₄OAc (370 mg, 4.75 mmol) in THF (5 mL) and H₂O (2 mL) was added zinc powder (380 mg, 5.85 mmol), the reaction mixture was stirred room temperature for 2 hours. The reaction mixture was filtered, the filtrate was acidified with 0.5N HCl to pH 3-4, and extracted with ethyl acetate (3×10 mL). The organic layer was washed by brine, dried over Na₂SO₄, filtered and concentrated under reduced pressure. The mixture was purified by flash column chromatography (0-20% tert-butyl methyl ether/petroleum ether) to give 2-methylene-4-oxo-4-(3-(4-(trifluoromethyl)phenyl)oxetan-3-yloxy)butanoic acid (450 mg, 70% yield) as white solid. LCMS m/z 331.0 (M+H)⁺ (ES⁺). ¹H NMR (400 MHz, DMSO-d6) δ: 12.81 (s, 1H), 7.80-7.72 (m, 4H), 6.19 (s, 1H), 5.82 (s, 1H), 4.91 (d, J=7.7 Hz, 2H), 4.82 (d, J=7.7 Hz, 2H), 3.50 (s, 2H).

### Intermediate 10-2-methylene-4-oxo-4-(1-(5-(trifluoromethyl)pyridin-2-yl)cyclobutoxy)butanoic acid

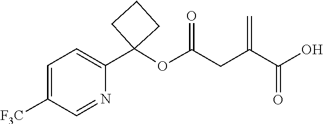 [SMILES: C=C(CC(=O)OC1(c2ccc(C(F)(F)F)cn2)CCC1)C(=O)O]

Prepared by an analogous method to Intermediate 9 starting from 2-bromo-5-(trifluoromethyl)pyridine (400 mg, 1.78 mmol) except that toluene was used as solvent in place of THF in Step 1. Yield: 102 mg, 0.31 mmol. White solid. LCMS m/z 330.0 (M+H)⁺ (ES⁺). ¹H NMR (400 MHz, DMSO-d6) δ: 12.74 (br, 1H), 8.98 (s, 1H), 8.14 (dd, J=8.5, 2.4 Hz, 1H), 7.59 (d, J=8.4 Hz, 1H), 6.16 (d, J=1.6 Hz, 1H), 5.79 (d, J=1.4 Hz, 1H), 3.41 (s, 2H), 2.73-2.62 (m, 2H), 2.50-2.41 (m, 2H), 2.03-1.85 (m, 2H).

### Intermediate 11—2-methylene-4-oxo-4-(3-(5-(trifluoromethyl)pyridin-2-yl)oxetan-3-yloxy)butanoic acid

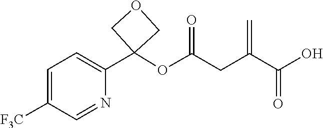 [SMILES: C=C(CC(=O)OC1(c2ccc(C(F)(F)F)cn2)COC1)C(=O)O]

Prepared by an analogous method to Intermediate 9 starting from 2-bromo-5-(trifluoromethyl)pyridine (3.0 g, 13.30 mmol) and oxetan-3-one (1.05 g, 14.63 mmol). Yield: 155 mg, 65%. White solid. LCMS m/z 331.9 (M+H)⁺ (ES⁺). ¹H NMR (400 MHz, DMSO-d6) δ: 12.83 (br, 1H), 9.08 (dt, J=2.1, 1.0 Hz, 1H), 8.22 (dd, J=8.5, 2.4, Hz, 1H), 7.68 (d, J=8.4 Hz, 1H), 6.21 (d, J=1.2 Hz, 1H), 5.86 (d, J=1.2 Hz, 1H), 4.99 (d, J=7.6 Hz, 2H), 4.84 (d, J=7.6 Hz, 2H), 3.56 (s, 2H).

### Intermediate 12—3-(((9H-fluoren-9-yl)methoxy)carbonyl)but-3-enoic acid

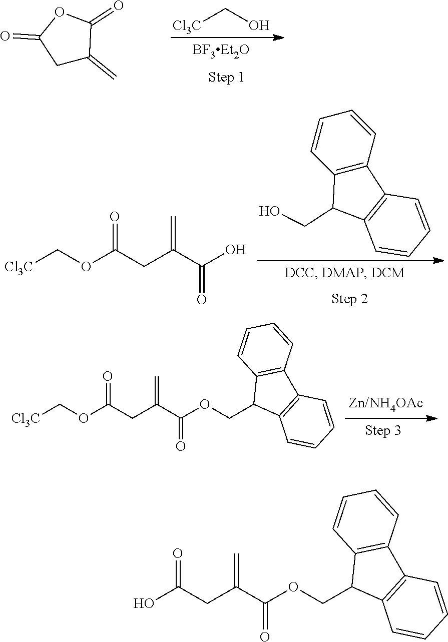

### Step 1

To a mixture of 3-methylenedihydrofuran-2,5-dione (10.0 g, 89.22 mmol) and 2,2,2-trichloroethanol (20.0 g, 133.82 mmol) was added boron trifluoride diethyl etherate (1.27 g, 8.92 mmol), and the mixture was allowed to stir at 75° C. for 40 minutes. The mixture was cooled to room temperature, quenched with methanol (4 mL), diluted with EtOAc (100 mL) and water (20 mL), separated and extracted with EtOAc (2×50 mL). The combined organic layers were washed with brine, dried over Na₂SO₄ and filtered. The filtrate was concentrated under reduced pressure, the residue was purified by reversed column chromatography (Column: Boston ODS 120 g Flash; Flow Rate: 40 mL/min; solvent system: MeCN/(10 mmol/L HCl/water); MeCN gradient: 60-80%; collection wavelength: 214 nm). The combined fractions were concentrated under reduced pressure to remove MeCN, and the residue was lyophilized to give 2-methylene-4-oxo-4-(2,2,2-trichloroethoxy)butanoic acid (18.0 g). The resulting solid was triturated in a mixture of n-hexane (150 mL) and tert-butyl methyl ether (20 mL), stirred at room temperature overnight, filtered and dried at 40° C. under reduced pressure to give 2-methylene-4-oxo-4-(2,2,2-trichloroethoxy)butanoic acid (16.0 g, 68% yield) as white solid. LCMS m/z 283.2 (M+Na)⁺ (ES⁺).

### Step 2

A mixture of 2-methylene-4-oxo-4-(2,2,2-trichloroethoxy)butanoic acid (13.0 g, 49.72 mmol), (9H-fluoren-9-yl)methanol (9.76 g, 49.72 mmol), DCC (15.36 g, 74.58 mmol) and DMAP (910 mg, 7.46 mmol) in DCM (200 mL) was stirred at room temperature for 30 minutes. The mixture was filtered, the filtrate was concentrated under reduced pressure, and the residue was purified by flash column chromatography (0-20% tert-butyl methyl ether/petroleum ether) to give 1-((9H-fluoren-9-yl)methyl) 4-(2,2,2-trichloroethyl) 2-methylenesuccinatee (12.0 g, 55% yield) as a white solid. LCMS m/z 461.0 (M+Na)⁺ (ES⁺).

### Step 3

A mixture of 1-((9H-fluoren-9-yl)methyl) 4-(2,2,2-trichloroethyl) 2-methylenesuccinate (12.0 g, 27.29 mmol), zinc powder (5.32 g, 81.87 mmol) and NH₄OAc (10.50 g, 136.45 mol) in THF (80 mL) and H₂O (20 mL) was stirred at 25° C. for 2 hours. The reaction mixture was filtered, and the filtrate was extracted with tert-butyl methyl ether (2×5 mL). The combined organics were washed with brine, dried over Na₂SO₄ and concentrated under reduced pressure. The residue was purified by flash column chromatography (20-40% tert-butyl methyl ether/petroleum ether) to give 3-(((9H-fluoren-9-yl)methoxy)carbonyl)but-3-enoic acid (8.41 g, 73% yield) as white solid. LCMS m/z 331.0 (M+Na)⁺ (ES⁺).

### Intermediate 13—4-(1-(3,5-dichlorophenyl)cyclobutoxy)-2-methylene-4-oxobutanoic acid

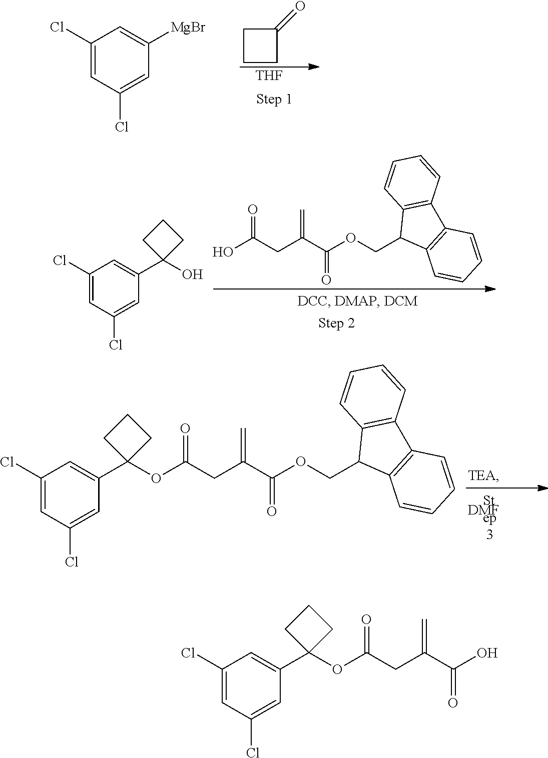

### Step 1

To the solution of cyclobutanone (1.40 g, 19.98 mmol) in THF (20 mL) was added (3,5-dichlorophenyl)magnesium bromide (20 mL, 9.99 mmol, 0.5M in 2-MeTHF) slowly at 0° C.; and the mixture was stirred at room temperature for 16 hour. After the reaction was completed (monitored by TLC), the reaction mixture was quenched with saturated ammonium chloride aqueous (30 mL), separated and the aqueous layer was extracted with ethyl acetate (20 mL×2). The combined organic layer was washed by brine, dried over Na₂SO₄, filtered and concentrated at 30° C. under reduced pressure to give 1-(3,5-dichlorophenyl)cyclobutanol (2.0 g, 92% yield) as colorless oil. ¹H NMR (400 MHz, CDCl₃) δ: 7.38 (d, J=2.0 Hz, 2H), 7.27 (t, J=2.0 Hz, 1H), 2.54-2.47 (m, 2H), 2.40-2.32 (m, 2H), 2.10-2.01 (m, 1H), 1.79-1.71 (m, 1H).

### Step 2

A mixture of 1-(3,5-dichlorophenyl)cyclobutanol (800 mg, 3.70 mmol), 3-(((9H-fluoren-9-yl)methoxy)carbonyl)but-3-enoic acid (1141 mg, 3.70 mmol), DCC (1143 mg, 5.55 mmol) and DMAP (45 mg, 0.37 mmol) in DCM (15 mL) was stirred at room temperature for 1 hour. After LCMS indicated the reaction completed, the mixture was filtered, and the filtrate was concentrated at 35° C. under reduced pressure, and the residue was purified by flash column chromatography (20 g, 0-15% tert-butyl methyl ether/petroleum ether) to give 1-(9H-fluoren-9-yl)methyl 4-(1-(3,5-dichlorophenyl)cyclobutyl) 2-methylenesuccinate (1.20 g, 64% yield) as a light yellow oil. LCMS m/z 528.8 (M+Na)⁺ (ES⁺).

### Step 3

A mixture of (1-(9H-fluoren-9-yl)methyl 4-(1-(3,5-dichlorophenyl)cyclobutyl) 2-methylenesuccinate (1.20 g, 2.37 mmol) in N,N-Dimethylformamide (6 mL) and triethylamine (1.2 mL) was stirred at 25° C. for 1 hour. After LCMS indicated the reaction was completed, the reaction mixture was acidified with 0.5N HCl until pH=6-7, and extracted with EtOAc (2×8 mL). The EtOAc layer was washed by brine, dried over Na₂SO₄, filtered and concentrated under reduced pressure, and the residue was purified by flash column chromatography (0-25% tert-butyl methyl ether/petroleum ether) to give 4-(1-(3,5-dichlorophenyl)cyclobutoxy)-2-methylene-4-oxobutanoic acid (650 mg, 83% yield) as white solid. LCMS m/z 351.0 (M+Na)⁺ (ES⁺).

### Example 1—N-(5-methylthiazol-2-yl)-2-((3-(1-(4-((trifluoromethyl)thio)phenyl)cyclopropyl)-1,2,4-oxadiazol-5-yl)methyl)acrylamide

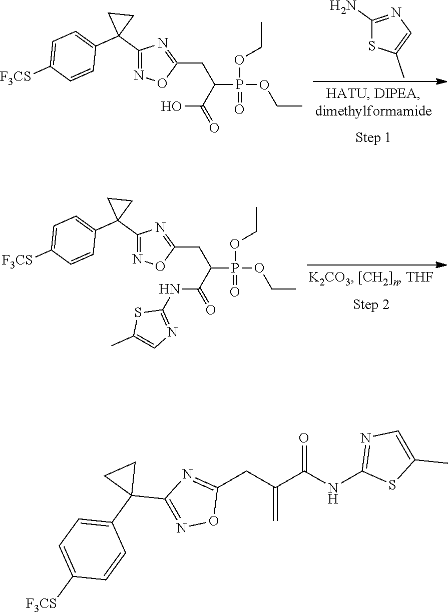

### Step 1

HATU (461 mg, 1.2 mmol) was added to a mixture of 5-methylthiazol-2-amine (139 mg, 1.2 mmol), 2-(diethoxyphosphoryl)-3-(3-(1-(4-((trifluoromethyl)thio)phenyl)cyclopropyl)-1,2,4-oxadiazol-5-yl)propanoic acid (Intermediate 2, 0.5 g, 1.0 mmol) and DIPEA (0.3 mL, 1.5 mmol) in dimethylformamide (2 mL). The mixture was stirred at RT for 16 h, then water (20 mL) was added. The mixture was extracted with EtOAc (3×10 mL). The combined organic phases were washed with sat. aq. bicarbonate (20 mL), 1 M hydrochloric acid (20 mL), brine (3×20 mL), dried (magnesium sulfate) and concentrated under reduced pressure. The crude product was purified by chromatography on silica (0-10% MeOH/DCM) to afford diethyl (1-((5-methylthiazol-2-yl)amino)-1-oxo-3-(3-(1-(4-((trifluoromethyl)thio)phenyl)cyclopropyl)-1,2,4-oxadiazol-5-yl)propan-2-yl)phosphonate (0.26 g, 0.3 mmol, 30% yield) as a light yellow oil. LCMS m/z 591.2 (M+H)⁺ (ES⁺).

### Step 2

Paraformaldehyde (13 mg, 0.4 mmol) was added to a suspension of diethyl (1-((5-methylthiazol-2-yl)amino)-1-oxo-3-(3-(1-(4-((trifluoromethyl)thio)phenyl)cyclopropyl)-1,2,4-oxadiazol-5-yl)propan-2-yl)phosphonate (159 mg, 0.2 mmol) and potassium carbonate (45 mg, 0.3 mmol) in THF (1.3 mL) and the mixture was heated to 50° C. for 1.5 h, then poured into water (15 mL). The mixture was extracted with EtOAc (3×15 mL) and the combined organic layers were washed with brine (20 mL), dried (magnesium sulfate) and concentrated under reduced pressure. The crude material was dissolved in DMSO (0.6 mL), filtered and purified by reversed phase preparative HPLC (column: Waters X-Select CSH C18 ODB; Flow Rate: 40 mL/min; solvent system: MeCN/(0.1% formic acid/water); MeCN gradient: 50-100%). The clean fractions were evaporated in a Genevac to give N-(5-methylthiazol-2-yl)-2-((3-(1-(4-((trifluoromethyl)thio)phenyl)cyclopropyl)-1,2,4-oxadiazol-5-yl)methyl)acrylamide (12 mg, 26 μmol, 13% yield) as a white solid. LCMS m/z 467.3 (M+H)⁺ (ES⁺). ¹H NMR (400 MHz, DMSO-d₆) δ 12.24 (br. s, 1H), 7.65-7.58 (m, 2H), 7.52-7.46 (m, 2H), 7.18 (d, J=1.4 Hz, 1H), 6.37 (s, 1H), 5.94 (s, 1H), 4.03 (s, 2H), 2.34 (d, J=1.3 Hz, 3H), 1.51-1.47 (m, 2H), 1.46-1.40 (m, 2H).

### Example 2—N-(pyrazin-2-yl)-2-((3-(1-(4-((trifluoromethyl)thio)phenyl)cyclopropyl)-1,2,4-oxadiazol-5-yl)methyl)acrylamide

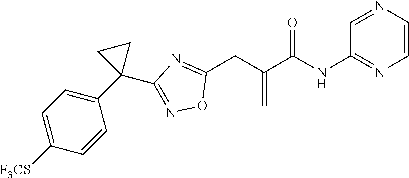

Prepared from 2-(diethoxyphosphoryl)-3-(3-(1-(4-((trifluoromethyl)thio)phenyl)cyclopropyl)-1,2,4-oxadiazol-5-yl)propanoic acid (Intermediate 2) and pyrazin-2-amine using a similar procedure to N-(5-methylthiazol-2-yl)-2-((3-(1-(4-((trifluoromethyl)thio)phenyl)cyclopropyl)-1,2,4-oxadiazol-5-yl)methyl)acrylamide.

### Step 1

LCMS m/z 572.1 (M+H)⁺ (ES+).

### Step 2

LCMS m/z 448.2 (M+H)⁺ (ES+). ¹H NMR (400 MHz, DMSO-d₆) δ 0.95 (s, 1H), 9.22 (d, J=1.6 Hz, 1H), 8.45 (dd, J=2.6, 1.6 Hz, 1H), 8.40 (d, J=2.5 Hz, 1H), 7.66-7.60 (m, 2H), 7.54-7.48 (m, 2H), 6.35 (s, 1H), 5.95 (s, 1H), 4.02 (s, 2H), 1.52-1.47 (m, 2H), 1.46-1.40 (m, 2H).

### Example 3—2-((3-(4-butylbenzyl)-1,2,4-oxadiazol-5-yl)methyl)-N-(pyridin-2-yl)acrylamide

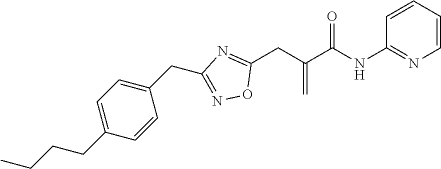 [SMILES: C=C(Cc1nc(Cc2ccc(CCCC)cc2)no1)C(=O)Nc1ccccn1]

Prepared from 3-(3-(4-butylbenzyl)-1,2,4-oxadiazol-5-yl)-2-(diethoxyphosphoryl) propanoic acid (Intermediate 3) and pyridin-2-amine using a similar procedure to N-(5-methylthiazol-2-yl)-2-((3-(1-(4-((trifluoromethyl)thio)phenyl)cyclopropyl)-1,2,4-oxadiazol-5-yl)methyl)acrylamide.

### Step 1

LCMS m/z 501.2 (M+H)⁺ (ES+).

### Step 2

LCMS m/z 377.2 (M+H)⁺ (ES+). ¹H NMR (400 MHz, DMSO-d₆) δ: 10.61 (br s, 1H), 8.35 (dm, J=4.9 Hz, 1H), 7.97 (d, J=8.4 Hz, 1H), 7.81-7.76 (m, 1H), 7.16-7.10 (m, 3H), 7.05 (d, J=8 Hz, 2H), 6.33 (s, 1H), 5.88 (s, 1H), 4.04 (s, 2H), 3.98 (s, 2H), 2.50-2.47 (m, 2H), 1.53-1.45 (m, 2H), 1.30-1.24 (m, 2H), 0.87 (t, J=7.2 Hz, 3H).

### Example 4—2-((3-(4-butylbenzyl)-1,2,4-oxadiazol-5-yl)methyl)-N-(pyrazin-2-yl)acrylamide

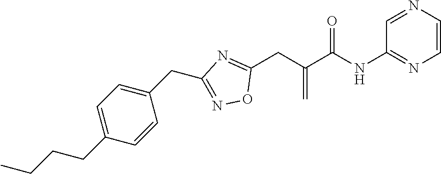 [SMILES: C=C(Cc1nc(Cc2ccc(CCCC)cc2)no1)C(=O)Nc1cnccn1]

Prepared from 3-(3-(4-butylbenzyl)-1,2,4-oxadiazol-5-yl)-2-(diethoxyphosphoryl) propanoic acid (Intermediate 3) and pyrazin-2-amine using a similar procedure to N-(5-methylthiazol-2-yl)-2-((3-(1-(4-((trifluoromethyl)thio)phenyl)cyclopropyl)-1,2,4-oxadiazol-5-yl)methyl)acrylamide.

### Step 1

LCMS m/z 502.1 (M+H)⁺ (ES+).

### Step 2

LCMS m/z 400.0 (M+Na)⁺ (ES+). ¹H NMR (400 MHz, DMSO-d₆) δ: 10.97 (br s, 1H), 9.21 (d, J=1.6 Hz, 1H), 8.45 (dd, J=2.4, 1.6 Hz, 1H), 8.39 (d, J=2.4 Hz, 1H), 7.13 (d, J=8.0 Hz, 2H), 7.04 (d, J=8.0 Hz, 2H), 6.37 (s, 1H), 5.96 (s, 1H), 4.03 (s, 2H), 3.98 (s, 2H), 2.50-2.47 (m, 2H), 1.53-1.43 (m, 2H), 1.31-1.22 (m, 2H), 0.88 (t, J=7.6 Hz, 3H).

### Example 5—2-((3-(4-butylbenzyl)-1,2,4-oxadiazol-5-yl)methyl)-N-(5-methylthiazol-2-yl)acrylamide

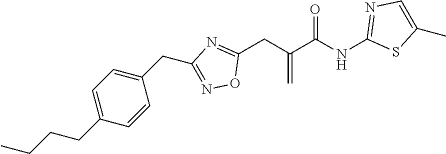 [SMILES: C=C(Cc1nc(Cc2ccc(CCCC)cc2)no1)C(=O)Nc1ncc(C)s1]

Prepared from 3-(3-(4-butylbenzyl)-1,2,4-oxadiazol-5-yl)-2-(diethoxyphosphoryl) propanoic acid (Intermediate 3) and 5-methylthiazol-2-amine using a similar procedure to N-(5-methylthiazol-2-yl)-2-((3-(1-(4-((trifluoromethyl)thio)phenyl)cyclopropyl)-1,2,4-oxadiazol-5-yl)methyl)acrylamide.

### Step 1

LCMS m/z 521.0 (M+H)⁺ (ES+).

### Step 2

LCMS m/z 419.0 (M+Na)⁺ (ES+). ¹H NMR (400 MHz, DMSO-d₆) δ: 12.26 (br s, 1H), 7.19 (s, 1H), 7.12 (d, J=8.4 Hz, 2H), 7.04 (d, J=8.4 Hz, 2H), 6.38 (s, 1H), 5.95 (s, 1H), 4.03 (s, 2H), 3.98 (s, 2H), 2.50-2.47 (m, 2H), 2.34 (s, 3H), 1.53-1.46 (m, 2H), 1.33-1.20 (m, 2H), 0.88 (t, J=7.2 Hz, 3H).

### Example 6—2-methyloctan-2-yl 3-(pyrazin-2-ylcarbamoyl)but-3-enoate

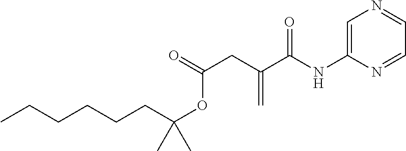 [SMILES: C=C(CC(=O)OC(C)(C)CCCCCC)C(=O)Nc1cnccn1]

Prepared from 2-(diethoxyphosphoryl)-4-((2-methyloctan-2-yl)oxy)-4-oxobutanoic acid (Intermediate 4) and pyrazin-2-amine using a similar procedure to N-(5-methylthiazol-2-yl)-2-((3-(1-(4-((trifluoromethyl)thio)phenyl)cyclopropyl)-1,2,4-oxadiazol-5-yl)methyl)acrylamide.

### Step 1

LCMS m/z 458.2 (M+H)⁺ (ES+).

### Step 2

LCMS m/z 334.1 (M+H)⁺ (ES+). ¹H NMR (400 MHz, DMSO-d₆) δ: 10.81 (s, 1H), 9.30 (d, J=1.2 Hz, 1H), 8.44 (m, 1H), 8.38 (d, J=2.4 Hz, 1H), 6.16 (s, 1H), 5.74 (s, 1H), 3.36 (s, 2H), 1.63-1.59 (m, 2H), 1.33 (s, 6H), 1.26-1.06 (m, 8H), 0.80 (t, J=6.8 Hz, 3H).

### Example 7—1-(4-(trifluoromethyl)phenyl)cyclobutyl 3-(methylcarbamoyl)but-3-enoate

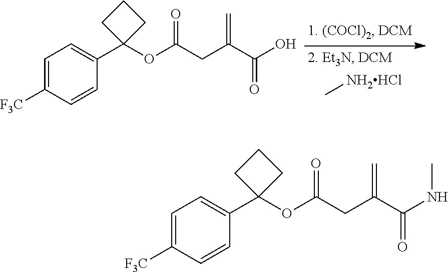

To a solution of 2-methylene-4-oxo-4-(1-(4-(trifluoromethyl)phenyl)cyclobutoxy)butanoic acid (600 mg, 1.83 mmol) in DCM (10 mL) was added oxalyl dichloride (346 mg, 2.75 mmol) at 0° C., and the reaction mixture was stirred at room temperature for 1 hour. The reaction mixture was concentrated under reduced pressure to give a yellow solid (600 mg crude). To a solution of methylamine hydrochloride (83 mg, 1.22 mmol) and Et₃N (246 mg, 2.44 mmol) in DCM (5 mL) was added the yellow solid synthesised above (200 mg) at 0° C., and the mixture was allowed to stir at room temperature for 1 hour. The mixture was quenched with H₂O (5 mL) and extracted with DCM (2×6 mL). The combined organic layers were washed with brine, dried over Na₂SO₄ and concentrated under reduced pressure. The residue was purified by prep-HPLC (Column: Waters SUNFIRE Prep C18 OBD 10 μm 19×250 mm; Flow Rate: 20 mL/min; solvent system: MeCN/(0.2% Formic/water; MeCN gradient: 55-95%; collection wavelength: 214 nm). The combined fractions were concentrated under reduced pressure to remove MeCN, and the residue was lyophilized to give 1-(4-(trifluoromethyl)phenyl)cyclobutyl 3-(methylcarbamoyl)but-3-enoate (34.60 mg, 16% yield) as white solid. LCMS m/z 364.0 (M+Na)⁺ (ES+). H NMR (400 MHz, DMSO-d₆) δ 8.10-8.03 (m, 1H), 7.71 (d, J=8.3 Hz, 2H), 7.64 (d, J=8.2 Hz, 2H), 5.76 (s, 1H), 5.47 (s, 1H), 3.29 (s, 2H), 2.63 (d, J=4.6 Hz, 3H), 2.57-2.47 (m, 4H), 2.03-1.89 (m, 1H), 1.84-1.67 (m, 1H).

### Example 8—1-(4-(trifluoromethyl)phenyl)cyclobutyl 3-carbamoylbut-3-enoate

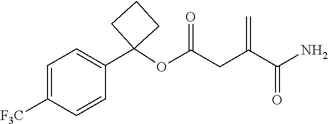 [SMILES: C=C(CC(=O)OC1(c2ccc(C(F)(F)F)cc2)CCC1)C(N)=O]

Prepared by an analogous method to Example 7 starting from 2-methylene-4-oxo-4-(1-(4-(trifluoromethyl)phenyl)cyclobutoxy)butanoic acid (600 mg, 1.83 mmol) and aqueous ammonia (2 mL). Yield: 50.3 mg, 25%. White solid. LCMS m/z 350.0 (M+Na)⁺ (ES⁺). ¹H NMR (400 MHz, DMSO-d6) δ: 7.70 (d, J=8.4 Hz, 2H), 7.65 (d, J=8.4 Hz, 2H), 7.60 (br. s, 1H), 7.07 (br. s, 1H), 5.87 (s, 1H), 5.50 (s, 1H), 3.26 (s, 2H), 2.55-2.50 (m, 4H), 2.02-1.91 (m, 1H), 1.85-1.70 (m, 1H).

### Example 9—(S)-1-(4-(trifluoromethyl)phenyl)ethyl 3-carbamoylbut-3-enoate

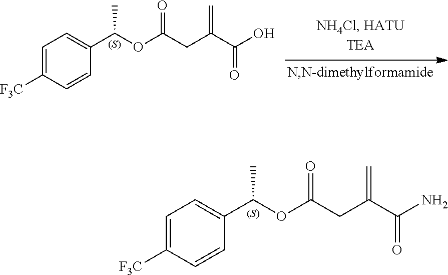

A mixture of (S)-2-methylene-4-oxo-4-(1-(4-(trifluoromethyl)phenyl)ethoxy)butanoic acid (300 mg, 0.99 mmol), ammonium chloride (104 mg, 2.0 mmol), HATU (456 mg, 1.2 mmol) and triethylamine (202 mg, 2 mmol) in N,N-dimethylformamide (3 mL) was stirred at room temperature for 1 hour. The mixture was quenched with H₂O (3 mL) and extracted with MTBE (3×5 mL). The combined organic layers were concentrated under reduced pressure and the residue was purified by prep-HPLC (Column: Waters SUNFIRE Prep C18 OBD 10 μm 19×250 mm; Flow Rate: 20 mL/min; solvent system: MeCN/(0.05% trifluoroacetic acid/water); MeCN gradient: 45-95%; collection wavelength: 214 nm). The combined fractions were concentrated under reduced pressure to remove MeCN, and the residue was lyophilized to give (S)-1-(4-(trifluoromethyl)phenyl)ethyl 3-carbamoylbut-3-enoate (227.4 mg, 57% yield) as white solid. LCMS m/z 324.2 (M+Na)⁺ (ES⁺). ¹H NMR (400 MHz, DMSO-d6) δ: 7.71 (d, J=8.1 Hz, 2H), 7.62 (br. s, 1H), 7.59 (d, J=8.1 Hz, 2H), 7.07 (br. s, 1H), 5.90 (s, 1H), 5.85 (q, J=6.6 Hz, 1H), 5.55 (s, 1H), 3.33 (s, 2H), 1.46 (d, J=6.6 Hz, 3H).

### Example 10—(S)-1-(4-(trifluoromethyl)phenyl)ethyl 3-(methylcarbamoyl)but-3-enoate

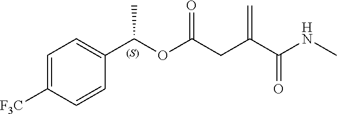

Prepared by an analogous method to Example 9 starting from (S)-2-methylene-4-oxo-4-(1-(4-(trifluoromethyl)phenyl)ethoxy)butanoic acid (360 mg, 1.19 mmol) and methanamine hydrochloride (161 mg, 2.38 mmol). Yield: 185.8 mg, 50%. Colorless oil. LCMS m/z 316.1 (M+H)⁺ (ES⁺). ¹H NMR (400 MHz, DMSO-d6) δ: 8.10-8.05 (m, 1H), 7.72 (d, J=8.1 Hz, 2H), 7.57 (d, J=8.1 Hz, 2H), 5.84 (q, J=6.6 Hz, 1H), 5.79 (s, 1H), 5.51 (s, 1H), 3.36 (s, 2H), 2.61 (d, J=4.5 Hz, 3H), 1.45 (d, J=6.6 Hz, 3H).

### Example 11—(R)-1-(4-(trifluoromethyl)phenyl)ethyl 3-(methylcarbamoyl)but-3-enoate

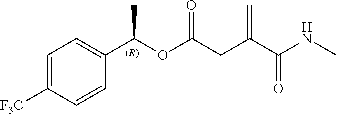

Prepared by an analogous method to Example 9 starting from (R)-2-methylene-4-oxo-4-(1-(4-(trifluoromethyl)phenyl)ethoxy)butanoic acid (200 mg, 0.66 mmol), and methanamine hydrochloride (50 mg, 0.73 mmol). Yield: 107.3 mg, 53%. White solid. LCMS m/z 316.2 (M+H)⁺ (ES⁺). ¹H NMR (400 MHz, DMSO-d6) δ: 8.11-8.06 (m, 1H), 7.72 (d, J=8.1 Hz, 2H), 7.57 (d, J=8.1 Hz, 2H), 5.85 (q, J=6.6 Hz, 1H), 5.79 (s, 1H), 5.51 (s, 1H), 3.36 (s, 2H), 2.61 (d, J=4.6 Hz, 3H), 1.45 (d, J=6.6 Hz, 3H).

### Example 12—(R)-1-(4-(trifluoromethyl)phenyl)ethyl 3-carbamoylbut-3-enoate

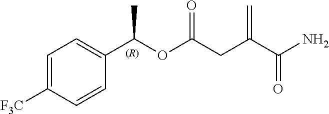

Prepared by an analogous method to Example 9 starting from (R)-2-methylene-4-oxo-4-(1-(4-(trifluoromethyl)phenyl)ethoxy)butanoic acid (200 mg, 0.66 mmol) and NH₄Cl (39 mg, 0.73 mmol). Yield: 44.0 mg, 22%. Colourless oil. LCMS m/z 324.1 (M+Na)⁺ (ES⁺). ¹H NMR (400 MHz, DMSO-d6) δ: 7.71 (d, J=8.0 Hz, 2H), 7.61 (br. s, 1H), 7.58 (d, J=8.0 Hz, 2H), 7.06 (br. s, 1H), 5.89 (s, 1H), 5.85 (q, J=6.6 Hz, 1H), 5.54 (s, 1H), 3.31 (s, 2H), 1.46 (d, J=6.6 Hz, 3H).

### Example 13—3-(4-(trifluoromethyl)phenyl)oxetan-3-yl 3-(methylcarbamoyl)but-3-enoate

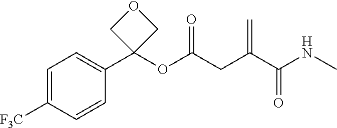 [SMILES: C=C(CC(=O)OC1(c2ccc(C(F)(F)F)cc2)COC1)C(=O)NC]

Prepared by an analogous method to Example 9 starting from 2-methylene-4-oxo-4-(3-(4-(trifluoromethyl)phenyl)oxetan-3-yloxy)butanoic acid (200 mg, 0.61 mmol) and methylamine hydrochloride (62 mg, 0.91 mmol). Yield: 93.4 mg, 45%. Colourless oil. m/z 366.1 (M+Na)⁺ (ES⁺). ¹H NMR (400 MHz, DMSO-d6) δ: 8.18-8.11 (m, 1H), 7.78 (d, J=8.8 Hz, 2H), 7.75 (d, J=8.8 Hz, 2H), 5.84 (s, 1H), 5.57 (s, 1H), 4.92 (d, J=8.0 Hz, 2H), 4.80 (d, J=8.0 Hz, 2H), 3.45 (s, 2H), 2.64 (d, J=4.4 Hz, 3H)

### Example 14—3-(4-(trifluoromethyl)phenyl)oxetan-3-yl 3-carbamoylbut-3-enoate

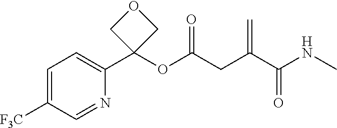 [SMILES: C=C(CC(=O)OC1(c2ccc(C(F)(F)F)cn2)COC1)C(=O)NC]

Prepared by an analogous method to Example 9 starting from 2-methylene-4-oxo-4-(3-(4-(trifluoromethyl)phenyl)oxetan-3-yloxy)butanoic acid (190 mg, 0.58 mmol) and ammonium chloride (46 mg, 0.87 mmol). Yield: 32.1 mg, 16%. Colourless oil. LCMS m/z 352.1 (M+Na)⁺ (ES⁺). ¹H NMR (400 MHz, DMSO-d6) δ: 7.76 (s, 4H), 7.68 (s, 1H), 7.13 (s, 1H), 5.94 (s, 1H), 5.61 (s, 1H), 4.92 (d, J=8.0 Hz, 2H), 4.79 (d, J=8.0 Hz, 2H), 3.43 (s, 2H).

### Example 15—3-(5-(trifluoromethyl)pyridin-2-yl)oxetan-3-yl 3-(methylcarbamoyl)but-3-enoate

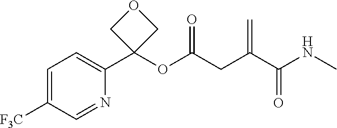 [SMILES: C=C(CC(=O)OC1(c2ccc(C(F)(F)F)cn2)COC1)C(=O)NC]

Prepared by an analogous method to Example 9 starting from 2-methylene-4-oxo-4-(3-(5-(trifluoromethyl)pyridin-2-yl)oxetan-3-yloxy)butanoic acid (150 mg, 0.45 mmol) and methylamine hydrochloride (46 mg, 0.68 mmol). Yield: 46.8 mg, 30%. White solid. LCMS m/z 345.3 (M+H)⁺ (ES⁺). ¹H NMR (400 MHz, DMSO-d6) δ: 9.07 (s, 1H), 8.24 (dd, J=8.5, 2.4 Hz, 1H), 8.20-8.14 (m, 1H), 7.72 (d, J=8.3 Hz, 1H), 5.87 (s, 1H), 5.60 (s, 1H), 4.98 (d, J=7.3 Hz, 2H), 4.84 (d, J=7.3 Hz, 2H), 3.50 (s, 2H), 2.65 (d, J=4.5 Hz, 3H).

### Example 16—3-(5-(trifluoromethyl)pyridin-2-yl)oxetan-3-yl 3-carbamoylbut-3-enoate

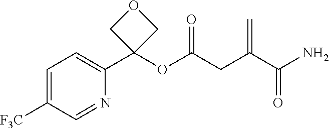 [SMILES: C=C(CC(=O)OC1(c2ccc(C(F)(F)F)cn2)COC1)C(N)=O]

Prepared by an analogous method to Example 9 starting from 2-methylene-4-oxo-4-(3-(5-(trifluoromethyl)pyridin-2-yl)oxetan-3-yloxy)butanoic acid (140 mg, 0.42 mmol) and ammonium chloride (34 mg, 0.63 mmol). Yield: 27.7 mg, 20%. White solid. LCMS m/z 331.1 (M+H)⁺ (ES⁺). ¹H NMR (400 MHz, DMSO-d6) δ: 9.09-9.06 (m, 1H), 8.20 (dd, J=8.4 Hz, 2.0 Hz, 1H), 7.72 (d, J=8.4 Hz, 1H), 7.70 (br. s, 1H), 7.14 (br. s, 1H), 5.97 (s, 1H), 5.64 (s, 1H), 4.97 (d, J=7.6 Hz, 2H), 4.83 (d, J=7.6 Hz, 2H), 3.47 (s, 2H).

### Example 17—1-(5-(trifluoromethyl)pyridin-2-yl)cyclobutyl 3-(methylcarbamoyl)but-3-enoate

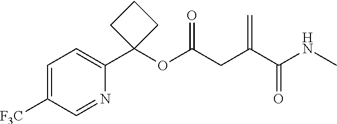 [SMILES: C=C(CC(=O)OC1(c2ccc(C(F)(F)F)cn2)CCC1)C(=O)NC]

Prepared by an analogous method to Example 9 starting from 2-methylene-4-oxo-4-(1-(5-(trifluoromethyl)pyridin-2-yl)cyclobutoxy)butanoic acid (200 mg, 0.61 mmol) and methylamine hydrochloride (62 mg, 0.91 mmol). Yield: 90.5 mg, 44%. White solid. LCMS m/z 343.1 (M+H)⁺ (ES⁺). ¹H NMR (400 MHz, DMSO-d6) δ: 8.99-8.96 (m, 1H), 8.16 (dd, J=8.6, 2.4 Hz, 1H), 8.13-8.07 (m, 1H), 7.60 (d, J=8.4 Hz, 1H), 5.80 (s, 1H), 5.53 (s, 1H), 3.37 (s, 2H), 2.69-2.65 (m, 2H), 2.64 (d, J=4.4 Hz, 3H), 2.49-2.43 (m, 2H), 2.01-1.88 (m, 2H).

### Example 18—1-(5-(trifluoromethyl)pyridin-2-yl)cyclobutyl 3-carbamoylbut-3-enoate

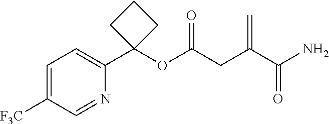 [SMILES: C=C(CC(=O)OC1(c2ccc(C(F)(F)F)cn2)CCC1)C(N)=O]

Prepared by an analogous method to Example 9 starting from 2-methylene-4-oxo-4-(1-(5-(trifluoromethyl)pyridin-2-yl)cyclobutoxy)butanoic acid (200 mg, 0.61 mmol) and NH₄Cl (36 mg, 0.67 mmol). Yield: 45.4 mg, 22%. White solid. LCMS m/z 329.2 (M+H)⁺ (ES⁺). ¹H NMR (400 MHz, DMSO-d6) δ: 8.99-8.96 (m, 1H), 8.12 (dd, J=8.4, 2.0 Hz, 1H), 7.66-7.59 (m, 2H), 7.09 (s, 1H), 5.91 (s, 1H), 5.57 (s, 1H), 3.34 (s, 2H), 2.69-2.62 (m, 2H), 2.46-2.43 (m, 2H), 2.02-1.87 (m, 2H).

### Example 19—1-(3,5-dichlorophenyl)cyclobutyl 3-(methylcarbamoyl)but-3-enoate

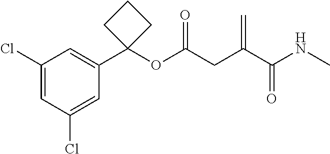 [SMILES: C=C(CC(=O)OC1(c2cc(Cl)cc(Cl)c2)CCC1)C(=O)NC]

Prepared by an analogous method to Example 9 starting from 4-(1-(3,5-dichlorophenyl)cyclobutoxy)-2-methylene-4-oxobutanoic acid (230 mg, 0.70 mmol) and methylamine hydrochloride (47 mg, 0.70 mmol). Yield: 103.9 mg, 43%. Yellow solid. LCMS (System 2, Method B) m/z 364.0 (M+Na)⁺ (ES⁺). ¹H NMR (400 MHz, DMSO-d6) δ: 88.11-8.03 (m, 1H), 7.52 (t, J=1.9 Hz, 1H), 7.42 (d, J=1.9 Hz, 2H), 5.78 (s, 1H), 5.48 (s, 1H), 3.30 (s, 2H), 2.64 (d, J=4.4 Hz, 3H), 2.58-2.55 (m, 2H), 2.51-2.42 (m, 2H), 1.99-1.87 (m, 1H), 1.81-1.68 (m, 1H).

### Example 20—1-(3,5-dichlorophenyl)cyclobutyl 3-carbamoylbut-3-enoate

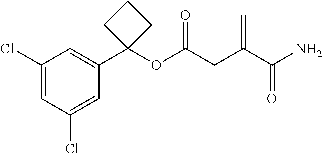 [SMILES: C=C(CC(=O)OC1(c2cc(Cl)cc(Cl)c2)CCC1)C(N)=O]

Prepared by an analogous method to Example 9 starting from 4-(1-(3,5-dichlorophenyl)cyclobutoxy)-2-methylene-4-oxobutanoic acid (230 mg, 0.70 mmol) and NH₄Cl (37 mg, 0.70 mmol). Yield: 35.8 mg, 15%. White solid. LCMS m/z 350.1 (M+Na)⁺ (ES⁺). ¹H NMR (400 MHz, DMSO-d6) δ: 7.60 (br. s, 1H), 7.51 (t, J=1.9 Hz, 1H), 7.43 (d, J=1.9 Hz, 2H), 7.06 (br. s, 1H), 5.89 (s, 1H), 5.52 (s, 1H), 3.27 (s, 2H), 2.56-2.41 (m, 4H), 1.96-1.91 (m, 1H), 1.78-1.71 (m, 1H).

### Example 21—1-(3-fluoro-4-(trifluoromethyl)phenyl)cyclobutyl 3-carbamoylbut-3-enoate

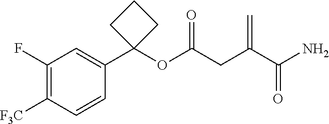 [SMILES: C=C(CC(=O)OC1(c2ccc(C(F)(F)F)c(F)c2)CCC1)C(N)=O]

Prepared by an analogous method to Example 9 starting from 4-(1-(3-fluoro-4-(trifluoromethyl)phenyl)cyclobutoxy)-2-methylene-4-oxobutanoic acid (200 mg, 0.58 mmol) and ammonium chloride (46 mg, 0.87 mmol). Yield: 86.6 mg, 44%. White solid. LCMS m/z 368.2 (M+Na)⁺ (ES⁺). ¹H NMR (400 MHz, DMSO-d6) δ: 7.75 (app. t, J=8.0 Hz, 1H), 7.62 (br. s, 1H), 7.53 (d, J=12.3 Hz, 1H), 7.47 (d, J=8.2 Hz, 1H), 7.09 (br. s, 1H), 5.89 (s, 1H), 5.53 (s, 1H), 3.29 (s, 2H), 2.56-2.50 (m, 4H), 2.02-1.90 (m, 1H), 1.88-1.75 (m, 1H).

### Example 22—1-(3-fluoro-4-(trifluoromethyl)phenyl)cyclobutyl 3-(methylcarbamoyl)but-3-enoate

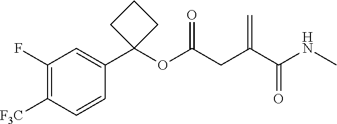 [SMILES: C=C(CC(=O)OC1(c2ccc(C(F)(F)F)c(F)c2)CCC1)C(=O)NC]

Prepared by an analogous method to Example 9 starting from 4-(1-(3-fluoro-4-(trifluoromethyl)phenyl)cyclobutoxy)-2-methylene-4-oxobutanoic acid (200 mg, 0.58 mmol) and methylamine hydrochloride (59 mg, 0.87 mmol). Yield: 86.1 mg, 42%. White solid. LCMS m/z 382.1 (M+Na)⁺ (ES⁺). ¹H NMR (400 MHz, DMSO-d6) δ: 8.08 (q, J=4.5 Hz, 1H), 7.76 (app. t, J=8.0 Hz, 1H), 7.51 (d, J=12.2 Hz, 1H), 7.46 (d, J=9.3 Hz, 1H), 5.78 (s, 1H), 5.49 (s, 1H), 3.32 (s, 2H), 2.63 (d, J=4.5 Hz, 3H), 2.59-2.45 (m, 4H), 2.02-1.90 (m, 1H), 1.86-1.74 (m, 1H).

### Example 23—1-(4-(trifluoromethyl)phenyl)cyclobutyl 3-((1-methylpiperidin-4-yl)carbamoyl)but-3-enoate

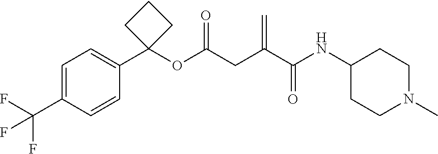 [SMILES: C=C(CC(=O)OC1(c2ccc(C(F)(F)F)cc2)CCC1)C(=O)NC1CCN(C)CC1]

Prepared by an analogous method to Example 9 starting from 2-methylene-4-oxo-4-(1-(4-(trifluoromethyl)phenyl)cyclobutoxy)butanoic acid (250 mg, 0.76 mmol) and 1-methylpiperidin-4-amine (87 mg, 0.76 mmol). Crude product was purified by prep-HPLC (Column: Waters SUNFIRE Prep C18 OBD 10 μm 19×250 mm; Flow Rate: 20 mL/min; solvent system: MeCN/(0.2% formic acid/water) gradient MeCN: 50-95%; collection wavelength: 214 nm). The fractions were concentrated under reduced pressure to remove MeCN, and lyophilized to give 1-(4-(trifluoromethyl)phenyl)cyclobutyl 3-(1-methylpiperidin-4-ylcarbamoyl)but-3-enoate, formate salt (96.2 mg, 30% yield) as light yellow oil. LCMS m/z 425.2 (M+H)⁺ (ES⁺). ¹H NMR (400 MHz, CDCl₃) δ: 8.29 (br. s, 1H), 7.61 (d, J=8.3 Hz, 2H), 7.53 (d, J=8.3 Hz, 2H), 6.40 (d, J=7.8 Hz, 1H), 5.81 (s, 1H), 5.46 (s, 1H), 3.98-3.93 (m, 1H), 3.45-3.35 (m, 2H), 3.33 (s, 2H), 2.78-2.72 (m, 2H), 2.71 (s, 3H), 2.66-2.59 (m, 4H), 2.14-1.95 (m, 5H), 1.83-1.73 (m, 1H).

### Example 24—1-(4-(trifluoromethyl)phenyl)cyclobutyl 3-((2-morpholinoethyl)carbamoyl)but-3-enoate

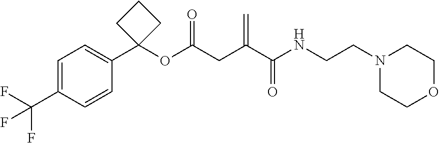 [SMILES: C=C(CC(=O)OC1(c2ccc(C(F)(F)F)cc2)CCC1)C(=O)NCCN1CCOCC1]

Prepared by an analogous method to Example 23 starting from 2-methylene-4-oxo-4-(1-(4-(trifluoromethyl)phenyl)cyclobutoxy)butanoic acid (150 mg, 0.46 mmol) and 2-morpholinoethanamine (59 mg, 0.46 mmol). Yield: 37.7 mg, 18%. Colourless oil. LCMS m/z 441.3 (M+H)⁺ (ES⁺). ¹H NMR (400 MHz, DMSO-d6) δ: 8.06 (t, J=5.7 Hz, 1H), 7.71 (d, J=8.1 Hz, 2H), 7.64 (d, J=8.2 Hz, 2H), 5.78 (s, 1H), 5.48 (s, 1H), 3.55-3.49 (m, 4H), 3.33 (s, 2H), 3.22 (q, J=6.6 Hz, 2H), 2.55-2.50 (m, 4H), 2.37-2.29 (m, 6H), 1.97-1.92 (m, 1H), 1.80-1.73 (m, 1H).

### Example 25—1-(4-(trifluoromethyl)phenyl)cyclobutyl 3-((1,1-dioxidothietan-3-yl)carbamoyl)but-3-enoate

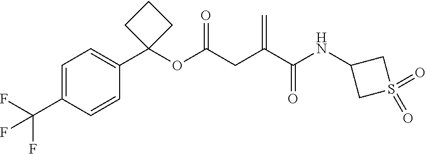 [SMILES: C=C(CC(=O)OC1(c2ccc(C(F)(F)F)cc2)CCC1)C(=O)NC1CS(=O)(=O)C1]

Prepared by an analogous method to Example 23 starting from 2-methylene-4-oxo-4-(1-(4-(trifluoromethyl)phenyl)cyclobutoxy)butanoic acid (150 mg, 0.46 mmol) and 3-aminothietane 1,1-dioxide (55 mg, 0.46 mmol). Yield: 106.5 mg, 54%. White solid. LCMS m/z 454.1 (M+Na)⁺ (ES⁺). ¹H NMR (400 MHz, DMSO-d6) δ: 8.78 (d, J=5.2 Hz, 1H), 7.72 (d, J=8.4 Hz, 2H), 7.63 (d, J=8.0 Hz, 2H), 5.89 (s, 1H), 5.60 (s, 1H), 4.54-4.48 (m, 2H), 4.41-4.34 (m, 1H), 4.14-4.09 (m, 2H), 3.32 (s, 2H), 2.57-2.52 (m, 4H), 1.99-1.92 (m, 1H), 1.79-1.72 (m, 1H).

### Example 26—1-(4-(trifluoromethyl)phenyl)cyclobutyl 3-((2-(methylsulfonyl)ethyl)carbamoyl)but-3-enoate

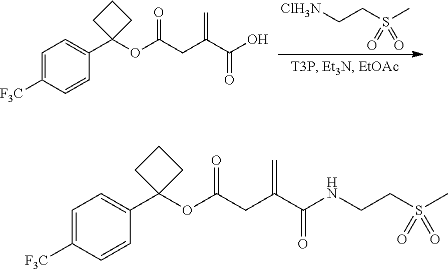

A solution of T3P (50 wt % in EtOAc, 0.8 mL, 1.3 mmol) was added to a solution of 2-methylene-4-oxo-4-(1-(4-(trifluoromethyl)phenyl)cyclobutoxy)butanoic acid (0.250 g, 0.66 mmol), 2-(methylsulfonyl)ethan-1-amine hydrochloride (106 mg, 0.66 mmol) and triethylamine (0.37 mL, 2.7 mmol) in EtOAc (3 mL) at RT. The mixture was stirred at RT for 1 h, then diluted with brine (20 mL) and extracted with EtOAc (2×20 mL). The combined organic phases were dried (MgSO₄) and concentrated. The crude product was purified by chromatography on RP Flash C18 (5-75% (0.1% Formic acid in MeCN)/(0.1% Formic Acid in Water)) to afford 1-(4-(trifluoromethyl)phenyl)cyclobutyl 3-((2-(methylsulfonyl)ethyl)carbamoyl)but-3-enoate (0.04 g, 0.09 mmol) as a colourless gum. LCMS m/z 456.1 (M+Na)⁺ (ES+). ¹H NMR (400 MHz, DMSO-d₆) δ: 8.38 (t, J=5.6 Hz, 1H), 7.71 (d, J=8.3 Hz, 2H), 7.64 (d, J=8.3 Hz, 2H), 5.81 (s, 1H), 5.53 (s, 1H), 3.58-3.47 (m, 2H), 3.32 (s, 2H), 3.25 (t, J=6.9 Hz, 2H), 2.97 (s, 3H), 2.57-2.52 (m, 4H), 2.03-1.88 (m, 1H), 1.85-1.67 (m, 1H).

### Example 27—(2-((3-(1-(4-((trifluoromethyl)thio)phenyl)cyclopropyl)-1,2,4-oxadiazol-5-yl)methyl)acryloyl)glycine

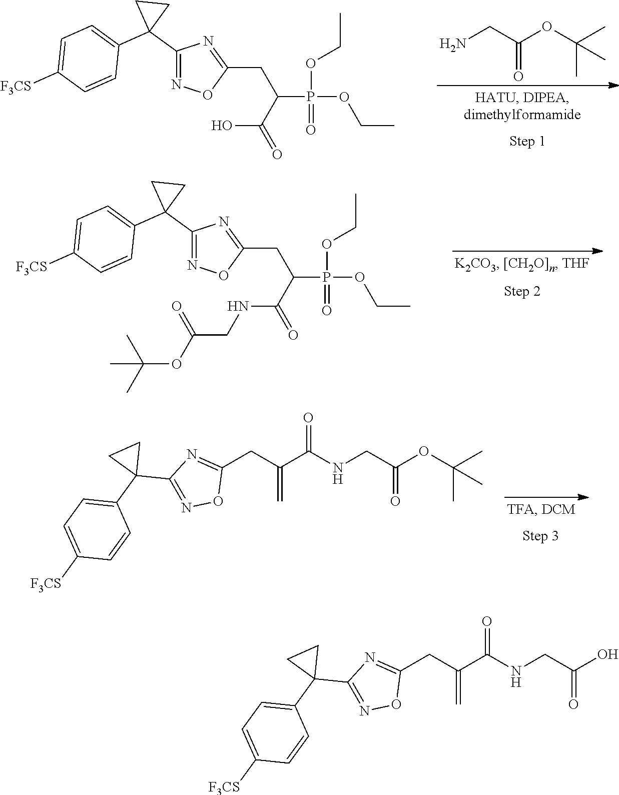

### Step 1

HATU (166 mg, 0.44 mmol) was added to a mixture of 2-(diethoxyphosphoryl)-3-(3-(1-(4-((trifluoromethyl)thio)phenyl)cyclopropyl)-1,2,4-oxadiazol-5-yl)propanoic acid (Intermediate 2, 0.20 g, 90% wt, 0.36 mmol), tert-butyl glycinate (68 μL, 0.50 mmol) and DIPEA (0.10 mL, 0.6 mmol) in N,N-dimethylformamide (2 mL). The mixture was stirred at RT for 2 h. The mixture was directly purified by chromatography on RP Flash C18 (5-75% (0.1% Formic acid in MeCN)/(0.1% Formic Acid in Water)) to afford tert-butyl (2-(diethoxyphosphoryl)-3-(3-(1-(4-((trifluoromethyl)thio)phenyl)cyclopropyl)-1,2,4-oxadiazol-5-yl)propanoyl)glycinate (0.17 g, 0.24 mmol, 85% purity) as a pale yellow gum. ¹H NMR (400 MHz, DMSO-de) δ 8.49 (t, J=5.7 Hz, 1H), 7.71-7.64 (m, 2H), 7.55-7.47 (m, 2H), 4.12-3.91 (m, 4H), 3.83-3.51 (m, 3H), 3.42-3.33 (m, 1H), 3.24-3.10 (m, 1H), 1.59-1.49 (m, 2H), 1.46-1.40 (m, 2H), 1.39 (s, 9H), 1.20 (dt, J=9.1, 7.0 Hz, 6H). LCMS m/z 552.2 (M-tBu+H)⁺ (ES+).

### Step 2

Paraformaldehyde (30 mg, 1.0 mmol) was added to a mixture of tert-butyl (2-(diethoxyphosphoryl)-3-(3-(1-(4-((trifluoromethyl)thio)phenyl)cyclopropyl)-1,2,4-oxadiazol-5-yl)propanoyl)glycinate (0.17 g, 85% purity, 0.24 mmol) and potassium carbonate (51 mg, 0.37 mmol) in THF (4 mL) at RT. The mixture was stirred at 50° C. for 2 h, then cooled to RT and poured into brine (10 mL). The mixture was extracted with DCM (3×10 mL) and the combined organic layers were dried (MgSO₄) and concentrated. The crude product was purified by chromatography on silica gel (0-100% MTBE/isohexane) to afford tert-butyl (2-((3-(1-(4-((trifluoromethyl)thio)phenyl)cyclopropyl)-1,2,4-oxadiazol-5-yl)methyl)acryloyl)glycinate (0.04 g, 0.08 mmol) as a colourless gum. LCMS m/z 428.3 (M-tBu+H)⁺ (ES+). ¹H NMR (400 MHz, DMSO-d₆) δ 8.61 (t, J=6.0 Hz, 1H), 7.72-7.64 (m, 2H), 7.56-7.48 (m, 2H), 6.01 (s, 1H), 5.74-5.68 (m, 1H), 3.88 (s, 2H), 3.73 (d, J=6.0 Hz, 2H), 1.55-1.48 (m, 2H), 1.46-1.41 (m, 2H), 1.39 (s, 9H).

### Step 3

TFA (0.06 mL, 0.8 mmol) was added to a solution of tert-butyl (2-((3-(1-(4-((trifluoromethyl)thio)phenyl)cyclopropyl)-1,2,4-oxadiazol-5-yl)methyl)acryloyl)glycinate (0.04 g, 0.08 mmol) in DCM (3 mL) at RT. The reaction was stirred at RT for 18 h, before additional TFA (0.06 mL, 0.8 mmol) was added and the mixture was stirred a further 18 h. The mixture was concentrated and the crude product was purified by chromatography on RP Flash C18 (5-75% (0.1% Formic acid in MeCN)/(0.1% Formic Acid in Water)) to afford (2-((3-(1-(4-((trifluoromethyl)thio)phenyl)cyclopropyl)-1,2,4-oxadiazol-5-yl)methyl)acryloyl)glycine (32 mg, 0.07 mmol) as a colourless gum. LCMS m/z 428.3 (M+H)⁺ (ES+). ¹H NMR (400 MHz, DMSO-d₆) δ 12.55 (s, 1H), 8.58 (t, J=5.9 Hz, 1H), 7.72-7.63 (m, 2H), 7.58-7.50 (m, 2H), 6.02 (s, 1H), 5.73-5.66 (m, 1H), 3.87 (s, 2H), 3.76 (d, J=5.9 Hz, 2H), 1.55-1.47 (m, 2H), 1.46-1.36 (m, 2H).

### Example 28—3,3,3-trifluoro-2-(2-((3-(1-(4-((trifluoromethyl)thio)phenyl)cyclopropyl)-1,2,4-oxadiazol-5-yl)methyl)acrylamido)propanoic a

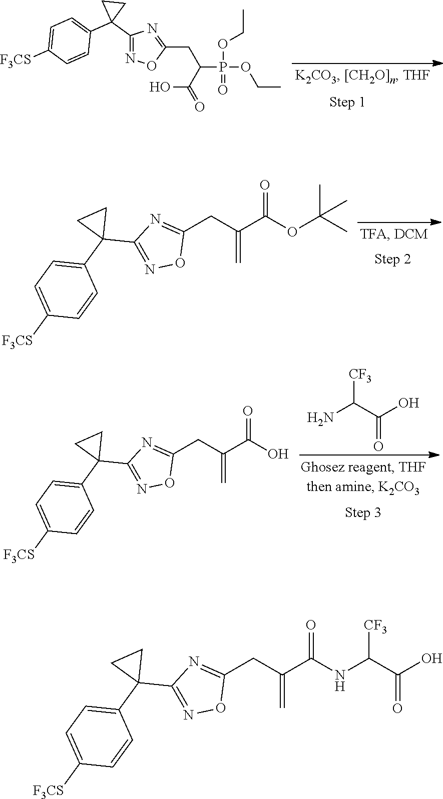

### Step 1

Aqueous formaldehyde (10 mL, 37% wt, 134 mmol) was added to a suspension of tert-butyl 2-(diethoxyphosphoryl)-3-(3-(1-(4-((trifluoromethyl)thio)phenyl)cyclopropyl)-1,2,4-oxadiazol-5-yl)propanoate (Intermediate 2, Step 3, 16.0 g, 27.6 mmol) and potassium carbonate (7.63 g, 55.2 mmol) in THF (100 mL) at RT. The mixture was stirred for 1 h, before water (10 mL) was added and the mixture was stirred for a further 2 h. The mixture was concentrated to ca. 30 mL, then diluted with water (100 mL) and extracted with EtOAc (3×100 mL). The combined organic phases were washed with brine (100 mL), dried (MgSO₄) and concentrated. The crude product was purified by chromatography on silica gel (0-50% MTBE/isohexane) to afford tert-butyl 2-((3-(1-(4-((trifluoromethyl)thio)phenyl)cyclopropyl)-1,2,4-oxadiazol-5-yl)methyl)acrylate (10.8 g, 24 mmol) as a colourless oil. LCMS m/z 371.1 (M-tBu+H)⁺ (ES⁺). ¹H NMR (400 MHz, DMSO-d6) δ 7.72-7.65 (m, 2H), 7.58-7.49 (m, 2H), 6.23-6.19 (m, 1H), 5.91-5.86 (m, 1H), 3.90 (s, 2H), 1.52-1.40 (m, 4H), 1.33 (s, 9H).

### Step 2

A mixture of tert-butyl 2-((3-(1-(4-((trifluoromethyl)thio)phenyl)cyclopropyl)-1,2,4-oxadiazol-5-yl)methyl)acrylate (10.8 g, 24 mmol) and TFA (20 mL, 0.260 mmol) in DCM (20 mL) was stirred at RT for 2 h, then concentrated. The residue was co-evaporated with toluene (3×30 mL). Hexane (30 mL) and MTBE (5 mL) were added and the mixture was triturated. The resulting solid was collected by filtration, washing with MTBE/hexane (1:10, ca. 50 mL). The crude product was purified by chromatography on RP Flash C18 (0-60% (0.1% Formic acid in MeCN)/(0.1% Formic Acid in Water)). The clean fractions were combined and concentrated. The residue was taken up in EtOAc (50 mL), dried (MgSO4) and concentrated to give a colourless oil. A mixture of MTBE/isohexane (1:10, 50 mL) was added and the mixture triturated. Further isohexane (ca. 100 mL) was added and the resulting solid was isolated by filtration, washing with isohexane (100 mL) to afford 2-((3-(1-(4-((trifluoromethyl)thio)phenyl)cyclopropyl)-1,2,4-oxadiazol-5-yl)methyl)acrylic acid (5.18 g, 14 mmol) as a white solid. LCMS m/z 371.0 (M+H)⁺ (ES⁺). ¹H NMR (400 MHz, DMSO-d6) δ 12.83 (s, 1H), 7.68 (d, J=8.1 Hz, 2H), 7.53 (d, J=8.3 Hz, 2H), 6.26 (s, 1H), 5.89 (s, 1H), 3.89 (s, 2H), 1.53-1.48 (m, 2H), 1.48-1.41 (m, 2H)

### Step 3

Ghosez reagent (0.11 mL, 0.83 mmol) was added to a solution of 2-((3-(1-(4-((trifluoromethyl)thio)phenyl)cyclopropyl)-1,2,4-oxadiazol-5-yl)methyl)acrylic acid (0.25 g, 0.68 mmol) in THF (5 mL) at 0° C. The mixture was stirred at RT for 1 h, then 2-amino-3,3,3-trifluoropropanoic acid (145 mg, 1.01 mmol), potassium carbonate (140 mg, 1.01 mmol) and water (1 mL) were added. The reaction mixture was stirred at RT for 18 h, then quenched with 1 M HCl (20 mL) and extracted with DCM (3×20 mL). The combined organic layers were dried (MgSO₄) and concentrated. The crude product was purified by chromatography on silica gel (0-60% MTBE/isohexane) to afford 3,3,3-trifluoro-2-(2-((3-(1-(4-((trifluoromethyl)thio)phenyl)cyclopropyl)-1,2,4-oxadiazol-5-yl)methyl)acrylamido)propanoic acid (0.03 g, 0.06 mmol) as a white solid. LCMS m/z 496.1 (M+H)⁺ (ES+). ¹H NMR (400 MHz, DMSO-d₆) δ: 13.89 (s, 1H), 9.21 (d, J=9.0 Hz, 1H), 7.72-7.63 (m, 2H), 7.58-7.48 (m, 2H), 6.16 (s, 1H), 5.84 (s, 1H), 5.32 (p, J=8.8 Hz, 1H), 3.93 (s, 2H), 1.56-1.36 (m, 4H).

### Example 29—(2-methylene-4-oxo-4-(1-(4-(trifluoromethyl)phenyl)cyclobutoxy)butanoyl) glycine

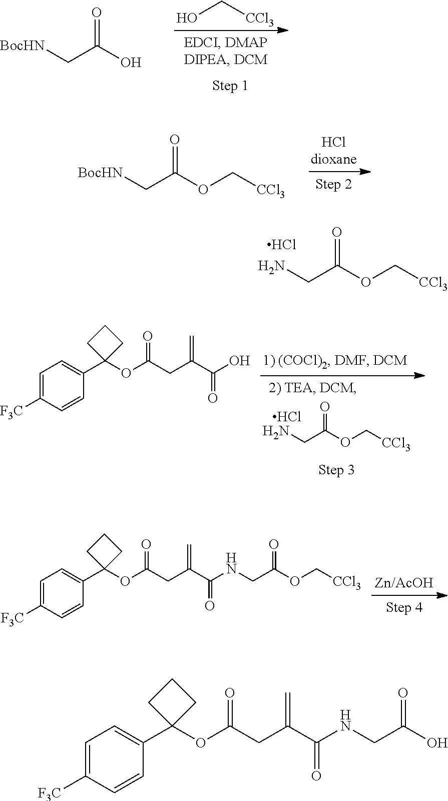

### Step 1

To a solution of (tert-butoxycarbonyl)glycine (6.0 g, 34.25 mmol), 2,2,2-trichloroethanol (5.63 g, 37.67 mmol), DMAP (418 mg, 3.42 mmol) and DIPEA (13.25 g, 102.75 mmol) in DCM (50 mL) was added EDCI (9.85 g, 51.37 mmol) at 0° C., and the resulting light yellow mixture was stirred at room temperature overnight. The mixture was quenched with 0.5 N HCl (20 mL) to pH 3-4 and extracted with DCM (3×30 mL). The combined organic layers were washed with brine, dried over Na₂SO₄ and the filtrate was concentrated under reduced pressure. The residue was purified by flash column chromatography (0-5% tert-butyl methyl ether/petroleum ether) to give 2,2,2-trichloroethyl (tert-butoxycarbonyl)glycinate (7.0 g, 67% yield) as a colorless oil. ¹H NMR (400 MHz, CDCl₃) δ: 5.01 (br. s, 1H), 4.78 (s, 2H), 4.06 (d, J=6.0 Hz, 2H), 1.44 (s, 9H).

### Step 2

A solution of 2,2,2-trichloroethyl (tert-butoxycarbonyl)glycinate (2.0 g, 6.52 mmol) in HCl/dioxane (10 mL, 4 mol/L) was stirred at room temperature for 3 hours. The mixture was concentrated under reduced pressure to give 2,2,2-trichloroethyl glycinate hydrochloride (1.50 g, quantitative yield) as a white solid. ¹H NMR (400 MHz, CDCl₃) δ: 8.52 (br. s, 3H), 5.02 (d, J=2.8 Hz, 2H), 4.00 (d, J=7.6 Hz, 2H).

### Step 3

To the solution of 2-methylene-4-oxo-4-(1-(4-(trifluoromethyl)phenyl)cyclobutoxy)butanoic acid (163 mg, 0.50 mmol) in DCM (2 mL) was added a drop of N,N-dimethylformamide and oxalyl chloride (77 mg, 0.60 mmol) at 0° C., and the mixture was stirred at room temperature for 30 minutes. The reaction mixture was concentrated under reduced pressure to give 1-(4-(trifluoromethyl)phenyl)cyclobutyl 3-(chlorocarbonyl)but-3-enoate (200 mg, quantitative yield) as light brown oil. To a mixture of 2,2,2-trichloroethyl glycinate hydrochloride (120 mg, 0.50 mmol) and triethyl amine (152 mg, 1.50 mmol) in DCM (2 mL) was added the solution of 1-(4-(trifluoromethyl)phenyl)cyclobutyl 3-(chlorocarbonyl)but-3-enoate (200 mg, 0.50 mmol) in DCM (2 mL) at 0° C., and the resulting light yellow mixture was stirred at room temperature for 3 hours. The mixture was quenched with 0.5 N HCl (5 mL) and extracted with DCM (3×5 mL). The combined organic layers were washed with brine, dried over Na₂SO₄ and the filtrate was concentrated under reduced pressure. The residue was purified by flash column chromatography (0-35% tert-butyl methyl ether/petroleum ether) to give 1-(4-(trifluoromethyl)phenyl)cyclobutyl 3-((2-oxo-2-(2,2,2-trichloroethoxy)ethyl)carbamoyl)but-3-enoate (160 mg, 63% yield) as a colorless oil. LCMS (System 2, Method C) m/z 537.8 (M+Na)⁺ (ES⁺).

### Step 4

To a solution of 1-(4-(trifluoromethyl)phenyl)cyclobutyl 3-((2-oxo-2-(2,2,2-trichloroethoxy)ethyl)carbamoyl)but-3-enoate (160 mg, 0.31 mmol) and zinc powder (101 mg, 1.55 mmol) in acetic acid (2 mL) was stirred at 30° C. for 24 hours. The reaction mixture was diluted with acetonitrile (5 mL), filtered and the filtrate was concentrated under reduced pressure. The residue was purified by prep-HPLC (Column: Waters SUNFIRE Prep C18 OBD 10 μm 19×250 mm; Flow Rate: 20 mL/min; solvent system: MeCN/(0.05% trifluoroacetic acid/water) gradient: MeCN: 45-95%; collection wavelength: 214 nm). The fractions were concentrated under reduced pressure to remove MeCN, and the residue was lyophilized to give (2-methylene-4-oxo-4-(1-(4-(trifluoromethyl)phenyl)cyclobutoxy)butanoyl)glycine (71.4 mg, 60% yield) as colorless oil. LCMS (System 2, Method B) m/z 407.9 (M+Na)⁺ (ES⁺). ¹H NMR (400 MHz, DMSO-d6) δ: 12.57 (br. s, 1H), 8.48 (t, J=5.6 Hz, 1H), 7.70 (d, J=8.4 Hz, 2H), 7.63 (d, J=8.4 Hz, 2H), 5.90 (s, 1H), 5.56 (s, 1H), 3.78 (d, J=6.0 Hz, 2H), 3.29 (s, 2H), 2.55-2.49 (m, 4H), 1.98-1.92 (m, 1H), 1.78-1.71 (m, 1H).

### Biological Example 1—THP-1 AlphaLISA IL-1β and IL-6 Cytokine Assay

Measuring Inhibitory Effects on IL-1β and IL-6 Cytokine Output from THP-1s

The cytokine inhibition profiles of compounds of formula (I) were determined in a differentiated THP-1 cell assay. All assays were performed in RPMI-1640 growth medium (Gibco), supplemented with 10% fetal bovine serum (FBS; Gibco), 1% penicillin-streptomycin and 1% sodium pyruvate unless specified otherwise. The IL-1β and IL-6 cytokine inhibition assays were each run in a background of differentiated THP-1 cells as described below. All reagents described were from Sigma-Aldrich unless specified otherwise. Compounds were prepared as 10 mM DMSO stocks.

### Assay Procedure

THP-1 cells were expanded as a suspension up to 80% confluence in appropriate growth medium. Cells were harvested, suspended, and treated with an appropriate concentration of phorbol 12-myristate 13-acetate (PMA) over a 72 hr period (37° C./5% CO₂).

Following 72 hrs of THP-1 cell incubation, cellular medium was removed and replaced with fresh growth media containing 1% of FBS. Working concentrations of compounds were prepared separately in 10% FBS treated growth medium and pre-incubated with the cells for 30 minutes (37° C./5% CO₂). Following the 30 minute compound pre-incubation, THP-1s were treated with an appropriate concentration of LPS and the THP-1s were subsequently incubated for a 24 hr period (37° C./5% CO₂). An appropriate final concentration of Nigericin was then dispensed into the THP-1 plates and incubated for 1 hour (37° C./5% CO₂) before THP-1 supernatants were harvested and collected in separate polypropylene 96-well holding plates.

Reagents from each of the IL-1β and IL-6 commercial kits (Perkin Elmer) were prepared and run according to the manufacturer's instructions. Subsequently, fluorescence signal detection in a microplate reader was measured (EnVision® Multilabel Reader, Perkin Elmer).

Percentage inhibition was calculated per cytokine by normalizing the sample data to the high and low controls used within each plate (+/−LPS respectively). Percentage inhibition was then plotted against compound concentration and the 50% inhibitory concentration (IC₅₀) was determined from the resultant concentration-response curve.

The data for all compounds of formula (I) tested in this assay are presented in Table 1 below. Dimethyl itaconate, 2-(2-chlorobenzyl)acrylic acid and 4-octyl itaconate were included as comparator compounds. Reference Example 1 corresponds to Example 192 in WO2020/222011 (Sitryx Therapeutics Limited) and is also used as a comparator compound, particularly for Example 6.

TABLE 1
THP-1 cell IL-1β and IL-6 IC₅₀ values (μM)

| Compound | IL-1β (IC₅₀) | IL-6 (IC₅₀) |
|---|---|---|
| dimethyl itaconate | >100 | >100 |
| 2-(2-chlorobenzyl)acrylic | >100 | NT* |
| acid | | |
| 4-octyl itaconate | 39.8 | 21.1 |
| Reference Example 1 | 18.4^{§} | 25.4 |
| Example 1 | 1.0 | 0.9 |
| Example 2 | 0.4 | 0.6 |
| Example 3 | 4.5 | NT |
| Example 4 | 1.1 | NT |
| Example 5 | 1.9 | NT |
| Example 6 | 3.8 | 2.1 |
| Example 7 | 20.5 | 12.1 |
| Example 8 | 34.3 | 13.9 |
| Example 9 | 10.0 | 4.5 |
| Example 10 | 10.5 | 3.5 |
| Example 11 | 3.3 | 1.9 |
| Example 12 | 20.0 | 14.4 |
| Example 13 | 17.5 | 2.6 |
| Example 14 | 6.5 | 4.4 |
| Example 15 | 13.1 | 10.9 |
| Example 16 | 8.6 | 4.6 |
| Example 17 | 14.4 | 4.8 |
| Example 18 | 9.3 | 14.4 |
| Example 19 | 17.5 | 3.3 |
| Example 20 | 13.2 | 8.3 |
| Example 21 | 15.4 | 6.9 |
| Example 22 | 11.0 | 9.4 |
| Example 23 | >31.6 | 24.1 |
| Example 24 | 8.6 | 6.8 |
| Example 25 | 19.9 | 9.5 |
| Example 26 | 18.0 | 11.3 |
| Example 27 | 9.5 | >100 |
| Example 28 | 7.9 | 16.2 |
| Example 29 | >100 | >31.6 |

*NT means not tested;
^{§}this mean value is different from that reported in WO2020/222011 because of additional repeat testing in the assays

These results reveal that compounds of formula (I) are expected to have anti-inflammatory activity as shown by their IC₅₀ values for inhibition of IL-1β and IL-6 release in this assay. Most compounds of the invention tested exhibited improved IL-1β and IL-6 lowering properties (IC₅₀ values) compared with dimethyl itaconate, 2-(2-chlorobenzyl)acrylic acid, 4-octyl itaconate and Reference Example 1. In addition, certain amide compounds of formula (I) may be more potent, as shown by the IL-1β and IL-6 IC₅₀ values, than equivalent ester compounds (see Example 6 compared with Reference Example 1 in Table 1).

### Biological Example 2—NRF2 Activation Assay

### Measuring Compound Activation Effects on the Anti-Inflammatory Transcription Factor NRF2 in DiscoverX PathHunter NRF2 Translocation Kit

Potency and efficacy of compounds of formula (I) against the target of interest to activate NRF2 (nuclear factor erythroid 2-related factor 2) were determined using the PathHunter NRF2 translocation kit (DiscoverX). The NRF2 translocation assay was run using an engineered recombinant cell line, utilising enzyme fragment complementation to determine activation of the Keap1-NRF2 protein complex and subsequent translocation of NRF2 into the nucleus. Enzyme activity was quantified using a chemiluminescent substrate consumed following the formation of a functional enzyme upon PK-tagged NRF2 translocation into the nucleus.

Additionally, a defined concentration of dimethyl fumarate was used as the ‘High’ control to normalise test compound activation responses.

### Assay Procedure

U2OS PathHunter eXpress cells were thawed from frozen prior to plating. Following plating, U2OS cells were incubated for 24 hrs (37° C./5% CO₂) in commercial kit provided cell medium.

Following 24 hrs of U2OS incubation, cells were directly treated with an appropriate final concentration of compound for −GSH conditions, or for +GSH conditions, an intermediate plate containing 6× working concentrations of compound stocks was prepared in a 6 mM working concentration of GSH solution (solubilised in sterile PBS). Following a 30 minute compound/GSH pre-incubation (37° C./5% CO_{Z}) for +GSH treatment, plated U2OS cells were incubated with an appropriate final concentration of compound or compound and GSH for the +GSH treatment.

Following compound (+/−GSH) treatment, the U20S plates were incubated for a further 6 hours (37° C./5% CO₂) before detection reagent from the PathHunter NRF2 commercial kit was prepared and added to test plates according to the manufacturer's instructions. Subsequently, the luminescence signal detection in a microplate reader was measured (PHERAstar®, BMG Labtech).

Percentage activation was calculated by normalising the sample data to the high and low controls used within each plate (+/−DMF). Percentage activation/response was then plotted against compound concentration and the 50% activation concentration (EC₅₀) was determined from the plotted concentration-response curve.

The data for all compounds of formula (I) tested in this assay are presented in Table 2 below. Dimethyl itaconate, 4-octyl itaconate and 2-(2-chlorobenzyl)acrylic acid were included as comparator compounds.

TABLE 2
NRF2 activation

| | −GSH | | +GSH | |
|---|---|---|---|---|
| Compound | EC₅₀ (μM) | Eₘₐₓ (%) | EC₅₀ (μM) | Eₘₐₓ (%) |
| dimethyl itaconate | 21.7 | 126 | 56.4 | 77 |
| 4-octyl itaconate | 22.1 | 165 | 28.9 | 148 |
| 2-(2-chlorobenzyl)acrylic | >100 | 2 | >100 | 9 |
| acid | | | | |
| Example 2 | 0.9 | 117 | 5.2 | 79 |
| Example 7 | 12.6 | 119 | >100 | 27 |
| Example 8 | >100 | 21 | >100 | 7 |
| Example 9 | 13.7 | 110 | >100 | 26 |
| Example 20 | 35.2 | 156 | >100 | 28 |
| Example 22 | 14.5 | 109 | >100 | 48 |
| Example 27 | >100 | 26 | >100 | 20 |
| Example 28 | 31.8 | 67 | >100 | 34 |

These results reveal that compounds of formula (I) are expected to have anti-inflammatory activity as shown by their EC₅₀ and Eₘₐₓ values for NRF2 activation in this assay. All examples tested exhibited higher potency and/or normalised percentage activation, as shown by lower EC₅₀ values and/or higher Eₘₐₓ in −GSH assay, compared to 2-(2-chlorobenzyl)acrylic acid. Preferred examples tested exhibited higher potency and/or normalised percentage activation, as shown by lower EC₅₀ values and/or higher Eₘₐₓ in −GSH assay, compared with dimethyl itaconate and 4-octyl itaconate.

### REFERENCES

The following publications cited in this specification are herein incorporated by reference in their entirety.
Ackermann et al. Proc. Soc. Exp. Bio. Med. 1949, 72(1), 1-9.
Andersen J. L. et al. Nat. Commun. 2018, 9, 4344.
Angiari S. and O'Neill L. A. Cell Res. 2018, 28, 613-615.
Bagavant G. et al. Indian J. Pharm. Sci. 1994, 56, 80-85.
Bambouskova M. et al. Nature 2018, 556, 501-504.
Blewett M. M. et al. Sci. Sign. 2016, 9 (445), rs10; 6.
Brennan M. S. et al. PLoS One 2015, 10, e0120254.
Brück J. et al. Exp. Dermatol. 2018, 27, 611-624.
Cocco M. et al. J. Med. Chem. 2014, 57, 10366-10382.
Cocco M. et al. J. Med. Chem. 2017, 60, 3656-3671.
Cordes T. et al. J. Biol. Chem. 2016, 291, 14274-14284.
Cordes T. et al. Mol. Metab. 2020, 32, 122-135.
Daly R. et al. medRxiv 2019, 19001594; doi: https://doi.org/10.1101/19001594.
Daniels B. P. et al. Immunity 2019, 50(1), 64-76.e4.
Dibbert S. et al. Arch. Dermatol. Res. 2013, 305, 447-451.
ElAzzouny M. et al. J. Biol. Chem. 2017, 292, 4766-4769.
Gillard G. O. et al. J. Neuroimmunol. 2015, 283, 74-85.
Gu L. et al. Immunol. Cell Biol. 2020 doi:10.1111/imcb.12316.
Hanke T. et al. Pharmacol. Therapeut. 2016, 157, 163-187.
Hooftman A. & O'Neill L. A. J. Trends in Immunology 2019, 40, 687-698.
Hunt T. et al. Consortium of Multiple Sclerosis Centers 2015 Annual Meeting, 27-30 May 2015,
Indianapolis, IN, USA: Poster DX37.
Kornberg M. D. et al. Science 2018, 360, 449-453.
Kulkarni R. A. et al. Nat. Chem. Biol. 2019 10.1038/s41589-018-0217-y.
Lampropoulou V. et al. Cell Metab. 2016, 24, 158-166.
Lehmann J. C. U. et al. J. Invest. Dermatol. 2007, 127, 835-845.
Liao S.-T. et al. Nat. Commun. 2019, 10(1), 5091.
Liu H. et al. Cell Commun. Signal. 2018, 16, 81.
McGuire V. A. et al. Sci. Rep. 2016, 6, 31159.
Michelucci A. et al. Proc. Natl. Acad. Sci. USA 2013, 110, 7820-7825.
Mills E. A. et al. Front. Neurol. 2018, 9, 5.
Mills E. L. et al. Cell 2016, 167, 457-470.
Mills E. L. et al. Nature 2018, 556, 113-117.
Mrowietz U. et al. Trends Pharmacol. Sci. 2018, 39, 1-12.
Müller S. et al. J. Dermatol. Sci. 2017, 87, 246-251.
Murphy M. P. and O'Neill L. A. J. Cell 2018, 174, 780-784.
Naismith R. T. et al., CNS Drugs 2020, 34, 185-196
O'Neill L. A. J. and Artyomov M. N. Nat. Rev. Immunol. 2019 273-281.
Olagnier D. et al. Nat. Commun. 2018, 9, 3506.
Schmidt T. J. et al. Bioorg. Med. Chem. 2007, 15, 333-342.
Shan Q. et al. Biochem. Biophys. Res. Commun. 2019, 517, 538-544.
Straub R. H. and Schradin C. Evol. Med. Public Health 2016, 1, 37-51S.
Straub R. H. and Cutolo M. Rheumatology 2016, 55 (Suppl. 2), ii6-ii14.
Sun X. et al., FASEB J. 2019, 33, 12929-12940.
Tang C. et al. Cell Physiol. Biochem. 2018, 51, 979-990.
Tang C. et al. Biochem. Biophys. Res. Commun. 2019, 508, 921-927.
Tang H. et al. Biochem. Biophys. Res. Commun. 2008, 375, 562-565.
Tian et al. Eur. J. Pharmacol. 2020, 873, 172989.
van der Reest J. et al. Nat. Commun. 2018, 9, 1581.
von Glehn F. et al. Mult. Scler. Relat. Disord. 2018, 23, 46-50.
Yi F. et al. Hepatology 2020, 873, 172989.
Yu X.-H. et al. Immunol. Cell Biol. 2019, 97, 134-141.
Zhang S. et al. Bioorg. Med. Chem. 2012, 20, 6073-6079.
Zhang D. et al. Int. Immunopharmacol. 2019, 77, 105924.
Zhang et al. Chem. Sci. 2021, 12, 6059.
Zhao C. et al. Microb. Pathogen. 2019, 133, 103541.
Zhao G. et al. Biochem. Biophys. Res. Commun. 2014, 448, 303-307.

### Miscellaneous

All references referred to in this application, including patent and patent applications, are incorporated herein by reference to the fullest extent possible.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims.

1. A compound of formula (I): 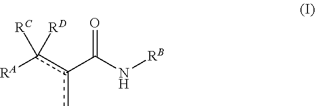
wherein,
R^{A} is: 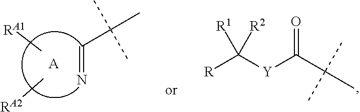
wherein when R^{A} is 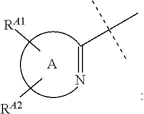
represents a 5 membered heteroaryl ring, which in addition to the C═N shown contains one or more further heteroatoms independently selected from N, O and S; or 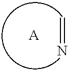
represents a 6 membered heteroaryl ring, which in addition to the C═N shown optionally contains one or more further N atoms;
R^{A1} is selected from the group consisting of C₁₋₁₀ alkyl, C₂₋₁₀ alkenyl, C₂₋₁₀ alkynyl, —(CH₂)₀₋₆—C₃₋₁₀ cycloalkyl, —(CH₂)₀₋₆—C₅₋₁₀ spirocycloalkyl, —(CH₂)₀₋₆-aryl and O-aryl;
wherein R^{A1} is optionally substituted by one or more R^{A′} wherein each R^{A′} is independently selected from the group consisting of halo, C₁₋₆ alkyl, C₁₋₆ haloalkyl, hydroxy, cyano, OG¹, S(O)₀₋₂G¹, SF₅, (CH₂)₀₋₃C₃₋₇ cycloalkyl and 5-7-membered heterocyclyl wherein said C₃₋₇ cycloalkyl and said 5-7-membered heterocyclyl are optionally substituted by one or more R^{A″} wherein each R^{A″} is independently selected from the group consisting of halo, C₁₋₃ alkyl and C₁₋₃ haloalkyl; wherein two alkyl groups which are attached to the same carbon atom are optionally joined to form a C₃₋₇ cycloalkyl ring; wherein the C₃₋₁₀ cycloalkyl group is optionally fused to a phenyl ring which phenyl ring is optionally substituted by one or more halo atoms; and/or R^{A1} is optionally substituted by one phenyl ring which is optionally substituted by C₁₋₂ haloalkyl, C₁₋₂ haloalkoxy or one or more halo atoms;
wherein, when two alkyl R^{A′} substituents are attached to a single carbon atom of R^{A1}, the two R^{A′} substituents may combine to form a 3- to 7-membered cycloalkyl ring;
wherein G¹ is C₁₋₆ alkyl, C₃₋₇ cycloalkyl, C₁₋₆ haloalkyl, or (CH₂)₀₋₁phenyl wherein G¹ is optionally substituted by one or more G^{1′} wherein G^{1′} is independently selected from the group consisting of halo, C₁₋₂ alkyl, C₁₋₂ haloalkyl, hydroxy, cyano, nitro, C₁₋₂ alkoxy and C₁₋₂ haloalkoxy;
R^{A2} is selected from the group consisting of halo, C₁₋₆ alkyl, C₂₋₆ alkenyl, C₂₋₆ alkynyl, C₁₋₆ haloalkyl, hydroxy, cyano, nitro, NR³R⁴, OG² and S(O)₀₋₂G²;
wherein G² is C₁₋₆ alkyl, C₃₋₇ cycloalkyl, C₁₋₆ haloalkyl, or phenyl which is optionally substituted by one or more G^{2′} wherein each G^{2′} is independently selected from the group consisting of halo, C₁₋₂ alkyl, C₁₋₂ haloalkyl, hydroxy, cyano, nitro, C₁₋₂ alkoxy and C₁₋₂ haloalkoxy; and
wherein R³ and R⁴ are independently H or C₁₋₂ alkyl or, taken together, R³ and R⁴ may combine to form a 5-7-membered heterocyclic ring;
or R^{A2} is absent;
wherein when R^{A} is 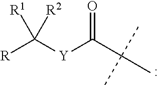
Y is O or NH; and
R is C₁₋₁₀ alkyl, and R¹ and R² are independently selected from the group consisting of H, C₁₋₄ alkyl and C₁₋₄ haloalkyl or R¹ and R² join to form a C₃₋₄ cycloalkyl ring; wherein R is optionally substituted by one or more R^{a} wherein each R^{a} is independently selected from the group consisting of halo, C₁₋₂ haloalkyl and C₁₋₂ haloalkoxy; or
R is selected from the group consisting of C₃₋₁₀ cycloalkyl, phenyl and 5- or 6-membered heteroaryl, and R¹ and R² are independently selected from the group consisting of H, C₁₋₄ alkyl and C₁₋₄ haloalkyl, or R¹ and R² join to form a C₃₋₄ cycloalkyl ring or a 4- to 6-membered heterocyclic ring, wherein the C₃₋₄ cycloalkyl ring or 4- to 6-membered heterocyclic ring is optionally substituted by methyl, halo or cyano; wherein R is optionally substituted by one or more R^{b} wherein each R^{b} is independently selected from the group consisting of halo, C₁₋₄ alkyl, C₁₋₄ haloalkyl, C₁₋₄ alkoxy, C₁₋₄ haloalkoxy and cyano; or
R is H, methyl or CF₃ and R¹ and R² are joined to form a C₃₋₁₀ cycloalkyl ring, wherein the C₃₋₁₀ cycloalkyl ring is optionally substituted by one or more R^{e} wherein each R^{e} is independently selected from the group consisting of halo, C₁₋₂ alkyl, C₁₋₂ haloalkyl, C₁₋₂ alkoxy and C₁₋₂ haloalkoxy, and/or wherein the C₃₋₁₀ cycloalkyl ring is optionally substituted by two R^{e} groups wherein the two R^{e} groups are attached to the same carbon atom and are joined to form a C₄₋₆ cycloalkyl ring;
R^{B} is heteroaryl wherein the heteroaryl is optionally substituted by one or more R^{B′} wherein each R^{B′} is independently selected from the group consisting of halo, C₁₋₂ alkyl, C₁₋₂ haloalkyl, C₁₋₂ alkoxy, C₁₋₂ haloalkoxy, CO₂H, CO₂C₁₋₄ alkyl and NR⁵R⁶, wherein R⁵ and R⁶ are independently H, C₁₋₄ alkyl and C₃₋₆ cycloalkyl, or R⁵ and R⁶ join to form a 4-7-membered heterocyclic ring; or
R^{B} is a 4- to 6-membered heterocyclic ring comprising one or two ring heteroatoms selected from N, O and S and optionally substituted on a ring nitrogen atom with C₁₋₂ alkyl and/or on a ring sulfur atom with one or two oxo substituents; or
R^{B} is selected from H, C₁₋₂ alkyl and C₁₋₂ haloalkyl, where alkyl and haloalkyl groups are optionally substituted with a substituent R^{B″};
wherein R^{B″} is selected from C(O)OH, C(O)O(C₁₋₂ alkyl), SO₂(C₁₋₂ alkyl), and a 5- or 6-membered heterocyclic ring comprising one or two ring heteroatoms selected from N, O and S and optionally substituted with C₁₋₂ alkyl; and
R^{C} and R^{D} are each independently H, C₁₋₂ alkyl, hydroxy, fluoro or C₁₋₂ alkoxy; or R^{C} and R^{D} may join to form a C₃₋₅ cycloalkyl ring;
wherein 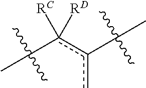
in the compound of formula (I) represents: 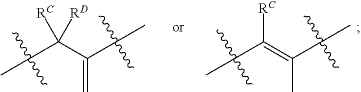
and wherein
the total number of carbon atoms in groups R^{A1} and R^{A2} taken together including their optional substituents is 1 to 14; and
wherein the total number of carbon atoms in groups R, R¹ and R² taken together, including their optional substituents, and including the carbon to which R, R¹ and R² are attached, is 3 to 14;
or a pharmaceutically acceptable salt and/or solvate thereof.

2. A compound according to claim 1 which is a compound of formula (IA): 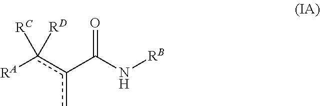
wherein,
R^{A} is 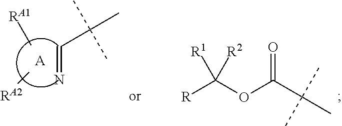
wherein when R^{A} is 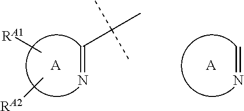
represents a 5 membered heteroaryl ring, which in addition to the C═N shown contains one or more further heteroatoms independently selected from N, O and S; or 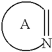
represents a 6 membered heteroaryl ring, which in addition to the C═N shown optionally contains one or more further N atoms;
R^{A1} is selected from the group consisting of C₁₋₁₀ alkyl, C₂₋₁₀ alkenyl, C₂₋₁₀ alkynyl, —(CH₂)₀₋₆—C₃₋₁₀ cycloalkyl, —(CH₂)₀₋₆—C₅₋₁₀ spirocycloalkyl, —(CH₂)₀₋₆-aryl and O-aryl;
wherein R^{A1} is optionally substituted by one or more R^{A′} wherein R^{A′} is independently selected from the group consisting of halo, C₁₋₆ alkyl, C₁₋₆ haloalkyl, hydroxy, cyano, OG¹, S(O)₀₋₂G¹, SF₅, (CH₂)₀₋₃C₃₋₇ cycloalkyl and 5-7-membered heterocyclyl wherein said C₃₋₇ cycloalkyl and said 5-7-membered heterocyclyl are optionally substituted by one or more R^{A″} wherein R^{A″} is independently selected from the group consisting of halo, C₁₋₃ alkyl and C₁₋₃ haloalkyl; wherein two alkyl groups which are attached to the same carbon atom are optionally joined to form a C₃₋₇ cycloalkyl ring; wherein the C₃₋₁₀ cycloalkyl group is optionally fused to a phenyl ring which phenyl ring is optionally substituted by one or more halo atoms; and/or R^{A1} is optionally substituted by one phenyl ring which is optionally substituted by C₁₋₂ haloalkyl, C₁₋₂ haloalkoxy or one or more halo atoms;
wherein G¹ is C₁₋₆ alkyl, C₃₋₇ cycloalkyl, C₁₋₆ haloalkyl, or (CH₂)₀₋₁phenyl wherein G¹ is optionally substituted by one or more G^{1′} wherein G^{1′} is independently selected from the group consisting of halo, C₁₋₂ alkyl, C₁₋₂ haloalkyl, hydroxy, cyano, nitro, C₁₋₂ alkoxy and C₁₋₂ haloalkoxy;
R^{A2} is selected from the group consisting of halo, C₁₋₆ alkyl, C₂₋₆ alkenyl, C₂₋₆ alkynyl, C₁₋₆ haloalkyl, hydroxy, cyano, nitro, NR³R⁴, OG² and S(O)₀₋₂G²;
wherein G² is C₁₋₆ alkyl, C₃₋₇ cycloalkyl, C₁₋₆ haloalkyl, or phenyl which is optionally substituted by one or more G^{2′} wherein G^{2′} is independently selected from the group consisting of halo, C₁₋₂ alkyl, C₁₋₂ haloalkyl, hydroxy, cyano, nitro, C₁₋₂ alkoxy and C₁₋₂ haloalkoxy; and
wherein R³ and R⁴ are independently H or C₁₋₂ alkyl or, taken together, R³ and R⁴ may combine to form a 5-7-membered heterocyclic ring;
or R^{A2} is absent; 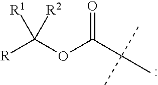
wherein when R^{A} is
R is C₁₋₁₀ alkyl, and R¹ and R² are independently selected from the group consisting of H, C₁₋₄ alkyl and C₁₋₄ haloalkyl or R¹ and R² join to form a C₃₋₄ cycloalkyl ring; wherein R is optionally substituted by one or more R^{a} wherein R^{a} is independently selected from the group consisting of halo, C₁₋₂ haloalkyl and C₁₋₂ haloalkoxy; or
R is selected from the group consisting of C₃₋₁₀ cycloalkyl, phenyl and 5- or 6-membered heteroaryl, and R¹ and R² are independently selected from the group consisting of H, C₁₋₄ alkyl and C₁₋₄ haloalkyl, or R¹ and R² join to form a C₃₋₄ cycloalkyl ring or a C₄₋₆ heterocycloalkyl ring, wherein the C₃₋₄ cycloalkyl ring is optionally substituted by methyl, halo or cyano; wherein R is optionally substituted by one or more R^{b} wherein R^{b} is independently selected from the group consisting of halo, C₁₋₄ alkyl, C₁₋₄ haloalkyl, C₁₋₄ alkoxy, C₁₋₄ haloalkoxy and cyano; or
R is H, methyl or CF₃ and R¹ and R² are joined to form a C₃₋₁₀ cycloalkyl ring, wherein the C₃₋₁₀ cycloalkyl ring is optionally substituted by one or more R^{e} wherein R^{e} is independently selected from the group consisting of halo, C₁₋₂ alkyl, C₁₋₂ haloalkyl, C₁₋₂ alkoxy and C₁₋₂ haloalkoxy, and/or wherein the C₃₋₁₀ cycloalkyl ring is optionally substituted by two R^{e} groups wherein the two R^{e} groups are attached to the same carbon atom and are joined to form a C₄₋₆ cycloalkyl ring;
R^{B} is heteroaryl wherein the heteroaryl is optionally substituted by one or more R^{B′} wherein R^{B′} is independently selected from the group consisting of halo, C₁₋₂ alkyl, C₁₋₂ haloalkyl, C₁₋₂ alkoxy, C₁₋₂ haloalkoxy, CO₂H, CO₂C₁₋₄ alkyl and NR⁵R⁶, wherein R⁵ and R⁶ are independently H, C₁₋₄ alkyl and C₃₋₆ cycloalkyl, or R⁵ and R⁶ join to form a 4-7-membered heterocyclic ring; and
R^{C} and R^{D} are each independently H, C₁₋₂ alkyl, hydroxy, fluoro or C₁₋₂ alkoxy; or R^{C} and R^{D} may join to form a C₃₋₅ cycloalkyl ring;
wherein 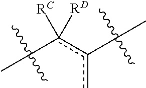
in the compound of formula (I) represents: 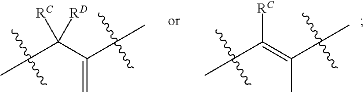
and wherein
the total number of carbon atoms in groups R^{A1} and R^{A2} taken together including their optional substituents is 1 to 14; and
wherein the total number of carbon atoms in groups R, R¹ and R² taken together, including their optional substituents, and including the carbon to which R, R¹ and R² are attached, is 3 to 14;
or a pharmaceutically acceptable salt and/or solvate thereof.

3. The compound, pharmaceutically acceptable salt and/or solvate thereof according to claim 1 wherein wherein R^{A} is 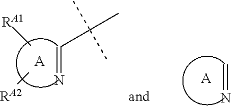
represents a 5 membered heteroaryl ring, which in addition to the C═N shown contains one or more further heteroatoms independently selected from N, O and S.

4. The compound, pharmaceutically acceptable salt and/or solvate thereof according to claim 1 wherein R^{A1} is —(CH₂)₀₋₆-aryl, wherein aryl is phenyl, wherein R^{A1} is optionally substituted by one or more (such as one, two or three) R^{A′} wherein R^{A′} is C₁₋₆ alkyl or SG¹, wherein G¹ is C₁₋₆ haloalkyl.

5. The compound, pharmaceutically acceptable salt and/or solvate thereof according to claim 4 wherein R^{A1} is —CH₂-aryl and wherein two methyl R^{A′} groups are attached to the CH₂ carbon of CH₂-aryl and are joined to form a cyclopropyl ring.

6. The compound, pharmaceutically acceptable salt and/or solvate thereof according to claim 1 wherein R^{A} is 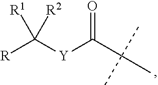
wherein Y is O or NH;
R is C₁₋₁₀ alkyl; and
R¹ and R² are independently selected from the group consisting of H, C₁₋₄ alkyl and C₁₋₄ haloalkyl or R¹ and R² join to form a C₃₋₄ cycloalkyl ring; or
Y is O or NH;
R is selected from C₃₋₁₀ cycloalkyl, phenyl and 5- or 6-membered heteroaryl; and
R¹ and R² are independently selected from the group consisting of H, C₁₋₄ alkyl and C₁₋₄ haloalkyl, or R¹ and R² join to form a C₃₋₄ cycloalkyl ring or a C₄₋₆ heterocyclic ring, wherein the C₃₋₄ cycloalkyl ring is optionally substituted by methyl, halo or cyano.

7. The compound, pharmaceutically acceptable salt and/or solvate thereof according to claim 6 wherein R is selected from C₃₋₁₀ cycloalkyl, phenyl and 5- or 6-membered heteroaryl and:
R¹ and R² are both H; or R¹ is and R² are both methyl; or
R¹ is H and R² is methyl; or
R¹ and R² join to form an unsubstituted cyclobutyl or oxetanyl ring.

8. The compound, pharmaceutically acceptable salt and/or solvate thereof according to claim 1 wherein R^{B} is selected from the group consisting of thiazolyl, pyridinyl, pyrimidinyl and pyrazinyl, wherein R^{B} is optionally substituted by methyl.

9. The compound, pharmaceutically acceptable salt and/or solvate thereof according to claim 1, wherein R^{B} is a 4- to 6-membered heterocyclic ring selected from the group consisting of piperidinyl, N-methyl piperidinyl, pyrrolidinyl, azetidinyl, morpholinyl, thietane-1,1-dioxide and thietane-1-oxide.

10. The compound, pharmaceutically acceptable salt and/or solvate thereof according to any one of claims 1 to 7 claim 1, wherein R^{B} is selected from the group consisting of H, C₁₋₂ alkyl or C₁₋₂ haloalkyl, wherein alkyl and haloalkyl groups are optionally substituted with a substituent R^{B″}, wherein R^{B″} is selected from C(O)OH, SO₂(methyl), and a 5- to 6-membered heterocyclic ring selected from piperidinyl, piperazinyl and morpholinyl.

11. The compound, pharmaceutically acceptable salt and/or solvate thereof according to claim 1, wherein R^{C} and R^{D} are each independently H.

12. The compound according to claim 1, which is selected from the list consisting of:
N-(5-methylthiazol-2-yl)-2-((3-(1-(4-((trifluoromethyl)thio)phenyl)cyclopropyl)-1,2,4-oxadiazol-5-yl)methyl)acrylamide;
N-(pyrazin-2-yl)-2-((3-(1-(4-((trifluoromethyl)thio)phenyl)cyclopropyl)-1,2,4-oxadiazol-5-yl)methyl)acrylamide;
2-((3-(4-butylbenzyl)-1,2,4-oxadiazol-5-yl)methyl)-N-(pyridin-2-yl)acrylamide;
2-((3-(4-butylbenzyl)-1,2,4-oxadiazol-5-yl)methyl)-N-(pyrazin-2-yl)acrylamide;
2-((3-(4-butylbenzyl)-1,2,4-oxadiazol-5-yl)methyl)-N-(5-methylthiazol-2-yl)acrylamide;
2-methyloctan-2-yl 3-(pyrazin-2-ylcarbamoyl)but-3-enoate;
1-(4-(trifluoromethyl)phenyl)cyclobutyl 3-(methylcarbamoyl)but-3-enoate;
1-(4-(trifluoromethyl)phenyl)cyclobutyl 3-carbamoylbut-3-enoate;
(S)-1-(4-(trifluoromethyl)phenyl)ethyl 3-carbamoylbut-3-enoate;
(S)-1-(4-(trifluoromethyl)phenyl)ethyl 3-(methylcarbamoyl)but-3-enoate;
(R)-1-(4-(trifluoromethyl)phenyl)ethyl 3-(methylcarbamoyl)but-3-enoate;
(R)-1-(4-(trifluoromethyl)phenyl)ethyl 3-carbamoylbut-3-enoate;
3-(4-(trifluoromethyl)phenyl)oxetan-3-yl 3-(methylcarbamoyl)but-3-enoate;
3-(4-(trifluoromethyl)phenyl)oxetan-3-yl 3-carbamoylbut-3-enoate;
3-(5-(trifluoromethyl)pyridin-2-yl)oxetan-3-yl 3-(methylcarbamoyl)but-3-enoate;
3-(5-(trifluoromethyl)pyridin-2-yl)oxetan-3-yl 3-carbamoylbut-3-enoate;
1-(5-(trifluoromethyl)pyridin-2-yl)cyclobutyl 3-(methylcarbamoyl)but-3-enoate;
1-(5-(trifluoromethyl)pyridin-2-yl)cyclobutyl 3-carbamoylbut-3-enoate;
1-(3,5-dichlorophenyl)cyclobutyl 3-(methylcarbamoyl)but-3-enoate;
1-(3,5-dichlorophenyl)cyclobutyl 3-carbamoylbut-3-enoate;
1-(3-fluoro-4-(trifluoromethyl)phenyl)cyclobutyl 3-carbamoylbut-3-enoate;
1-(3-fluoro-4-(trifluoromethyl)phenyl)cyclobutyl 3-(methylcarbamoyl)but-3-enoate;
1-(4-(trifluoromethyl)phenyl)cyclobutyl 3-((1-methylpiperidin-4-yl)carbamoyl)but-3-enoate;
1-(4-(trifluoromethyl)phenyl)cyclobutyl 3-((2-morpholinoethyl)carbamoyl)but-3-enoate;
1-(4-(trifluoromethyl)phenyl)cyclobutyl 3-((1,1-dioxidothietan-3-yl)carbamoyl)but-3-enoate;
1-(4-(trifluoromethyl)phenyl)cyclobutyl 3-((2-(methylsulfonyl)ethyl)carbamoyl)but-3-enoate;
(2-((3-(1-(4-((trifluoromethyl)thio)phenyl)cyclopropyl)-1,2,4-oxadiazol-5-yl)methyl)acryloyl) glycine;
3,3,3-trifluoro-2-(2-((3-(1-(4-((trifluoromethyl)thio)phenyl)cyclopropyl)-1,2,4-oxadiazol-5-yl)methyl)acrylamido)propanoic acid; and
(2-methylene-4-oxo-4-(1-(4-(trifluoromethyl)phenyl)cyclobutoxy)butanoyl) glycine;
or a pharmaceutically acceptable salt and/or solvate of any one thereof.

13. A pharmaceutical composition comprising the compound, pharmaceutically acceptable salt and/or solvate thereof according to claim 1 and a pharmacologically acceptable carrier or excipient.

14. A method of treating or preventing an inflammatory disease or a disease associated with an undesirable immune response, which comprises administering a compound, pharmaceutically acceptable salt and/or solvate thereof according to claim 1.

15. The according to claim 14, wherein the inflammatory disease or disease associated with an undesirable immune response is a disease selected from the group consisting of: psoriasis (including chronic plaque, erythrodermic, pustular, guttate, inverse and nail variants), asthma, chronic obstructive pulmonary disease (COPD, including chronic bronchitis and emphysema), heart failure (including left ventricular failure), myocardial infarction, angina pectoris, other atherosclerosis and/or atherothrombosis-related disorders (including peripheral vascular disease and ischaemic stroke), a mitochondrial and neurodegenerative disease (such as Parkinson's disease, Alzheimer's disease, Huntington's disease, amyotrophic lateral sclerosis, retinitis pigmentosa or mitochondrial encephalomyopathy), autoimmune paraneoplastic retinopathy, transplantation rejection (including antibody-mediated and T cell-mediated forms), multiple sclerosis, transverse myelitis, ischaemia-reperfusion injury (e.g. during elective surgery such as cardiopulmonary bypass for coronary artery bypass grafting or other cardiac surgery, following percutaneous coronary intervention, following treatment of acute ST-elevation myocardial infarction or ischaemic stroke, organ transplantation, or acute compartment syndrome), AGE-induced genome damage, an inflammatory bowel disease (e.g. Crohn's disease or ulcerative colitis), primary sclerosing cholangitis (PSC), PSC-autoimmune hepatitis overlap syndrome, non-alcoholic fatty liver disease (non-alcoholic steatohepatitis), rheumatica, granuloma annulare, cutaneous lupus erythematosus (CLE), systemic lupus erythematosus (SLE), lupus nephritis, drug-induced lupus, autoimmune myocarditis or myopericarditis, Dressler's syndrome, giant cell myocarditis, post-pericardiotomy syndrome, drug-induced hypersensitivity syndromes (including hypersensitivity myocarditis), eczema, sarcoidosis, erythema nodosum, acute disseminated encephalomyelitis (ADEM), neuromyelitis optica spectrum disorders, MOG (myelin oligodendrocyte glycoprotein) antibody-associated disorders (including MOG-EM), optic neuritis, CLIPPERS (chronic lymphocytic inflammation with pontine perivascular enhancement responsive to steroids), diffuse myelinoclastic sclerosis, Addison's disease, alopecia areata, ankylosing spondylitis, other spondyloarthritides (including peripheral spondyloarthritis, that is associated with psoriasis, inflammatory bowel disease, reactive arthritis or juvenile onset forms), antiphospholipid antibody syndrome, autoimmune hemolytic anaemia, autoimmune hepatitis, autoimmune inner ear disease, pemphigoid (including bullous pemphigoid, mucous membrane pemphigoid, cicatricial pemphigoid, herpes gestationis or pemphigoid gestationis, ocular cicatricial pemphigoid), linear IgA disease, Behçet's disease, celiac disease, Chagas disease, dermatomyositis, diabetes mellitus type I, endometriosis, Goodpasture's syndrome, Graves' disease, Guillain-Barre syndrome and its subtypes (including acute inflammatory demyelinating polyneuropathy, AIDP, acute motor axonal neuropathy (AMAN), acute motor and sensory axonal neuropathy (AMSAN), pharyngeal-cervical-brachial variant, Miller-Fisher variant and Bickerstaff s brainstem encephalitis), progressive inflammatory neuropathy, Hashimoto's disease, hidradenitis suppurativa, inclusion body myositis, necrotising myopathy, Kawasaki disease, IgA nephropathy, Henoch-Schonlein purpura, idiopathic thrombocytopenic purpura, thrombotic thrombocytopenic purpura (TTP), Evans' syndrome, interstitial cystitis, mixed connective tissue disease, undifferentiated connective tissue disease, morphea, myasthenia gravis (including MuSK antibody positive and seronegative variants), narcolepsy, neuromyotonia, pemphigus vulgaris, pernicious anaemia, psoriatic arthritis, polymyositis, primary biliary cholangitis (also known as primary biliary cirrhosis), rheumatoid arthritis, palindromic rheumatism, schizophrenia, autoimmune (meningo-)encephalitis syndromes, scleroderma, Sjogren's syndrome, stiff person syndrome, polymylagia rheumatica, giant cell arteritis (temporal arteritis), Takayasu arteritis, polyarteritis nodosa, Kawasaki disease, granulomatosis with polyangitis (GPA; formerly known as Wegener's granulomatosis), eosinophilic granulomatosis with polyangiitis (EGPA; formerly known as Churg-Strauss syndrome), microscopic polyarteritis/polyangiitis, hypocomplementaemic urticarial vasculitis, hypersensitivity vasculitis, cryoglobulinemia, thromboangiitis obliterans (Buerger's disease), vasculitis, leukocytoclastic vasculitis, vitiligo, acute disseminated encephalomyelitis, adrenoleukodystrophy, Alexander's disease, Alper's disease, balo concentric sclerosis or Marburg disease, cryptogenic organising pneumonia (formerly known as bronchiolitis obliterans organizing pneumonia), Canavan disease, central nervous system vasculitic syndrome, Charcot-Marie-Tooth disease, childhood ataxia with central nervous system hypomyelination, chronic inflammatory demyelinating polyneuropathy (CIDP), diabetic retinopathy, globoid cell leukodystrophy (Krabbe disease), graft-versus-host disease (GVHD) (including acute and chronic forms, as well as intestinal GVHD), hepatitis C (HCV) infection or complication, herpes simplex viral infection or complication, human immunodeficiency virus (HIV) infection or complication, lichen planus, monomelic amyotrophy, cystic fibrosis, pulmonary arterial hypertension (PAH, including idiopathic PAH), lung sarcoidosis, idiopathic pulmonary fibrosis, paediatric asthma, atopic dermatitis, allergic dermatitis, contact dermatitis, allergic rhinitis, rhinitis, sinusitis, conjunctivitis, allergic conjunctivitis, keratoconjunctivitis sicca, dry eye, xerophthalmia, glaucoma, macular oedema, diabetic macular oedema, central retinal vein occlusion (CRVO), macular degeneration (including dry and/or wet age related macular degeneration, AMD), post-operative cataract inflammation, uveitis (including posterior, anterior, intermediate and pan uveitis), iridocyclitis, scleritis, corneal graft and limbal cell transplant rejection, gluten sensitive enteropathy (coeliac disease), dermatitis herpetiformis, eosinophilic esophagitis, achalasia, autoimmune dysautonomia, autoimmune encephalomyelitis, autoimmune oophoritis, autoimmune orchitis, autoimmune pancreatitis, aortitis and periaortitis, autoimmune retinopathy, autoimmune urticaria, (idiopathic) Castleman's disease, Cogan's syndrome, IgG4-related disease, retroperitoneal fibrosis, juvenile idiopathic arthritis including systemic juvenile idiopathic arthritis (Still's disease), adult-onset Still's disease, ligneous conjunctivitis, Mooren's ulcer, pityriasis lichenoides et varioliformis acuta (PLEVA, also known as Mucha-Habermann disease), multifocal motor neuropathy (MMN), paediatric acute-onset neuropsychiatric syndrome (PANS) (including paediatric autoimmune neuropsychiatric disorders associated with streptococcal infections (PANDAS)), paraneoplastic syndromes (including paraneoplastic cerebellar degeneration, Lambert-Eaton myaesthenic syndrome, limbic encephalitis, brainstem encephalitis, opsoclonus myoclonus ataxia syndrome, anti-NMDA receptor encephalitis, thymoma-associated multiorgan autoimmunity), perivenous encephalomyelitis, reflex sympathetic dystrophy, relapsing polychondritis, sperm & testicular autoimmunity, Susac's syndrome, Tolosa-Hunt syndrome, Vogt-Koyanagi-Harada Disease, anti-synthetase syndrome, autoimmune enteropathy, immune dysregulation polyendocrinopathy enteropathy X-linked (IPEX), microscopic colitis, autoimmune lymphoproliferative syndrome (ALPS), autoimmune polyendocrinopathy-candidiasis-ectodermal dystrophy syndrome (APEX), gout, pseudogout, amyloid (including AA or secondary amyloidosis), eosinophilic fasciitis (Shulman syndrome) progesterone hypersensitivity (including progesterone dermatitis), amilial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutieres syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria), Schnitzler syndrome; familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders (e.g. myocardial infarction, angina, ischaemic heart failure, ischaemic nephropathy, ischaemic stroke, peripheral vascular disease, aortic aneurysm), and renal inflammatory disorders (e.g. diabetic nephropathy, membranous nephropathy, minimal change disease, crescentic glomerulonephritis, acute kidney injury, renal transplantation).

16. The method according to claim 14, for use in combination with a further therapeutic agent, such as a corticosteroid (glucocorticoid), retinoid (e.g. acitretin, isotretinoin, tazarotene), anthralin, vitamin D analogue (e.g. cacitriol, calcipotriol), calcineurin inhibitors (e.g. tacrolimus, pimecrolimus), phototherapy or photochemotherapy (e.g. psoralen ultraviolet irradiation, PUVA) or other form of ultraviolet light irradiation therapy, ciclosporine, a thiopurine (e.g. azathioprine, 6-mercaptopurine), methotrexate, an anti-TNFα agents (e.g. infliximab, etanercept, adalimumab, certolizumab, golimumab or a biosimilar), phosphodiesterase-4 (PDE4) inhibition (e.g. apremilast, crisaborole), anti-IL-17 agent (e.g. brodalumab, ixekizumab, secukinumab), anti-IL12/IL-23 agent (e.g. ustekinumab, briakinumab), anti-IL-23 agent (e.g. guselkumab, tildrakizumab), JAK (Janus Kinase) inhibitor (e.g. tofacitinib, ruxolitinib, baricitinib, filgotinib, upadacitinib), plasma exchange, intravenous immune globulin (IVIG), cyclophosphamide, anti-CD20 B cell depleting agent (e.g. rituximab, ocrelizumab, ofatumumab, 22iponimod22ab), anthracycline analogue (e.g. mitoxantrone), cladribine, sphingosine 1-phosphate receptor modulator or sphingosine analogue (e.g. fingolimod, 22iponimod, ozanimod, etrasimod), interferon beta preparation (including interferon beta 1b/1a), glatiramer, anti-CD3 therapy (e.g. OKT3), anti-CD52 targeting agent (e.g. alemtuzumab), leflunomide, teriflunomide, gold compound, laquinimod, potassium channel blocker (e.g. dalfampridine/4-aminopyridine), mycophenolic acid, mycophenolate mofetil, purine analogue (e.g. pentostatin), mTOR (mechanistic target of rapamycin) pathway inhibitor (e.g. sirolimus, everolimus), anti-thymocyte globulin (ATG), IL-2 receptor (CD25) inhibitor (e.g. basiliximab, daclizumab), anti-IL-6 receptor or anti-IL-6 agent (e.g. tocilizumab, siltuximab), Bruton's tyrosine kinase (BTK) inhibitor (e.g. ibrutinib), tyrosine kinase inhibitor (e.g. imatinib), ursodeoxycholic acid, hydroxychloroquine, chloroquine, B cell activating factor (BAFF, also known as BlyS, B lymphocyte stimulator) inhibitor (e.g. belimumab, blisibimod), other B cell targeted therapy including a fusion protein targeting both APRIL (A Proliferation-Inducing Ligand) and BlyS (e.g. atacicept), PI3K inhibitor including pan-inhibitor or one targeting the p1106 and/or p1107 containing isoforms (e.g. idelalisib, copanlisib, duvelisib), an interferon α receptor inhibitor (e.g. anifrolumab, sifalimumab), T cell co-stimulation blocker (e.g. abatacept, belatacept), thalidomide and its derivatives (e.g. lenalidomide), dapsone, clofazimine, a leukotriene antagonist (e.g. montelukast), theophylline, anti-IgE therapy (e.g. omalizumab), an anti-IL-5 agent (e.g. mepolizumab, reslizumab), a long-acting muscarinic agent (e.g. tiotropium, aclidinium, umeclidinium), a PDE4 inhibitor (e.g. roflumilast), riluzole, a free radical scavenger (e.g. edaravone), a proteasome inhibitor (e.g. bortezomib), a complement cascade inhibitor including one directed against C5 (e.g. eculizumab), immunoadsor, antithymocyte globulin, 5-aminosalicylates and their derivatives (e.g. sulfasalazine, balsalazide, mesalamine), an anti-integrin agent including one targeting α4β1 and/or α4β7 integrins (e.g. natalizumab, vedolizumab), an anti-CD11-α agent (e.g. efalizumab), a non-steroidal anti-inflammatory drug (NSAID) including a salicylate (e.g. aspirin), a propionic acid (e.g. ibuprofen, naproxen), an acetic acid (e.g. indomethacin, diclofenac, etodolac), an oxicam (e.g. meloxicam) a fenamate (e.g. mefenamic acid), a selective or relatively selective COX-2 inhibitor (e.g. celecoxib, etroxicoxib, valdecoxib and etodolac, meloxicam, nabumetone), colchicine, an IL-4 receptor inhibitor (e.g. dupilumab), topical/contact immunotherapy (e.g. diphenylcyclopropenone, squaric acid dibutyl ester), anti-IL-1 receptor therapy (e.g. anakinra), IL-10 inhibitor (e.g. canakinumab), IL-1 neutralising therapy (e.g. rilonacept), chlorambucil, a specific antibiotic with immunomodulatory properties and/or ability to modulate NRF2 (e.g. tetracyclines including minocycline, clindamycin, macrolide antibiotics), anti-androgenic therapy (e.g. cyproterone, spironolactone, finasteride), pentoxifylline, ursodeoxycholic acid, obeticholic acid, fibrate, a cystic fibrosis transmembrane conductance regulator (CFTR) modulator, a VEGF (vascular endothelial growth factor) inhibitor (e.g. bevacizumab, ranibizumab, pegaptanib, aflibercept), pirfenidone or mizoribine.

17. A process for preparing a compound of formula (I) according to claim 1, or a salt such as a pharmaceutical acceptable salt thereof, which comprises either:
reacting a compound of formula (II): 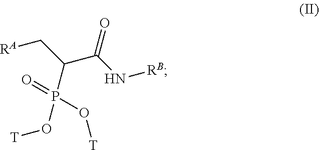
or a salt thereof;
with formaldehyde or a formaldehyde equivalent thereof, e.g., paraformaldehyde;
wherein R^{A} and R^{B} are defined in claim 1, and T is C₁₋₄ alkyl such as ethyl; or
reacting a compound of formula (XV): 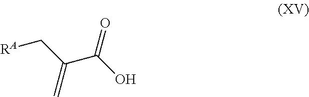
wherein R^{A} is as defined in claim 1;
with a halogenating agent to produce an acyl halide and reacting the acyl halide with a compound of formula (III):
H₂N—R^{B} (III)
wherein R^{B} is as defined in claim 1; or
reacting a compound of formula (XV) as defined above with a compound of formula (III) in the presence of a coupling agent and a base.

18. A compound of formula (II): 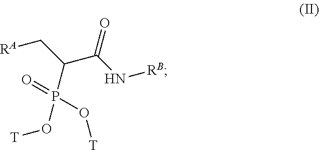
or a salt thereof;
wherein R^{A} and R^{B} are defined in claim 1, and T is C₁₋₄ alkyl such as ethyl; or
a compound of formula (XV): 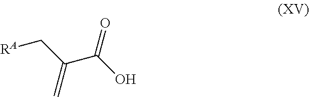
wherein R^{A} is as defined in claim 1.
